(19) United States
(12) Patent Application Publication
(10) Pub. No.: US 2022/0299511 A1
(43) Pub. Date: Sep. 22, 2022

(54) IMMUNE CELL SIGNATURE FOR BACTERIAL SEPSIS

(71) Applicants: The Broad Institute, Inc., Cambridge, MA (US); Massachusetts Institute of Technology, Cambridge, MA (US); The General Hospital Corporation, Boston, MA (US)

(72) Inventors: Miguel Reyes, Boston, MA (US); Nir Hacohen, Boston, MA (US); Paul C. Blainey, Cambridge, MA (US); Michael R. Filbin, Boston, MA (US); Marcia B. Goldberg, Boston, MA (US); Roby P. Bhattacharyya, Boston, MA (US)

(73) Assignees: The Broad Institute, Inc., Cambridge, MA (US); Massachusetts Institute of Technology, Cambridge, MA (US); The General Hospital Corporation, Boston, MA (US)

(21) Appl. No.: 17/619,958

(22) PCT Filed: Jun. 17, 2020
(86) PCT No.: PCT/US20/38244

§ 371 Date: Dec. 16, 2021

Related U.S. Application Data
(60) Provisional application No. 62/862,587, filed on Jun. 17, 2019.

(51) Int. Cl. G01N 33/569; A61P 31/00

(52) U.S. Cl. CPC G01N 33/56972; A61P 31/00; G01N 2333/70596; G01N 2800/26

(57) ABSTRACT

Provided herein, in some embodiments, are methods for analyzing immune cells in a blood sample from a subject having, suspected of having, or being at risk for bacterial sepsis. The present disclosure is based, at least in part, on the finding that certain immune cells are expanded in subjects having sepsis compared to healthy subjects.

### CROSS REFERENCE TO RELATED APPLICATIONS

This application claims the benefit under 35 U.S.C. § 119(e) of U.S. Provisional Application No. 62/862,587, filed Jun. 17, 2019, entitled “Immune Cell Signature For Bacterial Sepsis,” the entire disclosure of which is hereby incorporated by reference.

### FEDERALLY SPONSORED RESEARCH

This invention was made with government support under Grant Nos. AI118668 and AI119157, awarded by the National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jun. 17, 2020, is named B119570079WO00-SEQ-OMJ.txt, and is 11.0 kilobytes in size.

### FIELD OF THE INVENTION

The present disclosure relates to methods for identifying and treating subjects having, suspected of having, or being at risk for having sepsis.

### BACKGROUND

The human immune response to bacterial infection is complex and involves the coordinated action of several immune cell types both locally and systemically. Dysregulation of this response can lead to sepsis, which involves a dysregulated host response to infection that leads to organ damage. Sepsis is a prevalent disease with high mortality, and a major contributor to healthcare spending worldwide.

### SUMMARY

The present disclosure is based, at least in part, on the finding that certain immune cells are expanded in subjects having sepsis compared to healthy subjects.

Aspects of the disclosure relate to methods for treating a subject for sepsis, comprising:

administering an antibiotic to a subject who has been identified as having elevated levels of CD45+ monocytes that are IL1R2^{hi}, HLA-DR^{lo}, and CD14+ relative to a control. Further aspects of the disclosure relate to methods for treating a subject for sepsis, comprising: identifying a subject as having elevated levels of CD45+ monocytes that are IL1R2^{hi}, HLA-DR^{lo}, and CD14+ relative to a control; and administering an antibiotic to the subject.

Further aspects of the disclosure relate to methods comprising: measuring the fraction of CD45+ monocytes that are IL1R2^{hi}, HLA-DR^{lo}, and CD14+ in a blood sample from a subject; and comparing the fraction of CD45+ monocytes that are IL1R2^{hi}, HLA-DR^{lo}, and CD14+ in the blood sample from the subject to a control.

Further aspects of the disclosure relate to: methods for determining whether a subject has bacterial sepsis, comprising measuring the fraction of CD45+ monocytes that are IL1R2^{hi}, HLA-DR^{lo}, and CD14+ in a blood sample from the subject; comparing the fraction of CD45+ monocytes that are IL1R2^{hi}, HLA-DR^{lo}, and CD14+ in the blood sample from the subject to a control; and determining that the subject has bacterial sepsis if the fraction of CD45+ monocytes that are IL1R2^{hi}, HLA-DR^{lo}, and CD14+ in the blood sample from the subject is elevated compared to the control.

In some embodiments, methods further comprise determining that the subject has bacterial sepsis if the fraction of CD45+ monocytes that are IL1R2^{hi}, HLA-DR^{lo}, and CD14+ in the blood sample from the subject is elevated compared to a control.

In some embodiments, the control is a blood sample from a healthy subject. In some embodiments, the control is a predetermined value.

In some embodiments, methods further comprise administering an antibiotic to the subject.

In some embodiments, identifying a subject as having elevated levels of CD45+ monocytes that are IL1R2^{hi}, HLA-DR^{lo}, and CD14+ relative to a control comprises conducting an RNA-sequencing assay. In some embodiments, measuring the fraction of CD45+ monocytes that are IL1R2^{hi}, HLA-DR^{lo}, and CD14+ comprises conducting an RNA-sequencing assay. In some embodiments, the RNA-sequencing assay comprises a single cell RNA-sequencing (scRNA-seq) assay.

In some embodiments, identifying a subject as having elevated levels of CD45+ monocytes that are IL1R2^{hi}, HLA-DR^{lo}, and CD14+ relative to a control comprises conducting a flow cytometry assay. In some embodiments, measuring the fraction of CD45+ monocytes that are IL1R2^{hi}, HLA-DR^{lo}, and CD14+ comprises conducting a flow cytometry assay. In some embodiments, the flow cytometry assay comprises a fluorescence activated cell sorting (FACS) assay.

In some embodiments, the blood sample comprises total CD45+ monocytes and enriched dendritic cells. In some embodiments, the blood sample is obtained from a human.

In some embodiments, the subject is a human patient having, suspected of having, or at risk for a bacterial infection. In some embodiments, the subject is a human patient having, suspected of having, or at risk for bacterial sepsis.

In some embodiments, the bacterial infection is associated with a bacteria selected from the group consisting of Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria; Staphylococcus; Streptococcus; and Salmonella.

In some embodiments, the bacterial sepsis is associated with a bacteria selected from the group consisting of Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria; Staphylococcus; Streptococcus; and Salmonella.

In some embodiments, the subject is a human patient having, suspected of having, or at risk for a urinary tract infection (UTI).

Further aspects of the disclosure relate to methods for determining whether a subject has bacterial sepsis, comprising measuring the level of RETN, IL1R2, and/or CLU in CD14+ monocytes in a blood sample from the subject; comparing the level of RETN, IL1R2, and/or CLU in CD14+ monocytes in the blood sample from the subject to a control; and determining that the subject has bacterial sepsis if the level of RETN, IL1R2, and/or CLU in CD14+ monocytes in the blood sample from the subject is elevated relative to a control.

Further aspects of the disclosure relate to methods of identifying a sepsis condition in a subject comprising identifying an elevated fraction of MS1 type monocytes in the subject.

Further aspects of the disclosure relate to methods of identifying and treating a sepsis condition in a subject comprising identifying an elevated fraction of MS1 type monocytes in the subject, and treating the subject having elevated MS1 type monocytes by administering one or more antibiotic agents to the subject.

In some embodiments, the MS1 type monocytes are CD14+ monocytes characterized by high expression of RETN, IL1R2, and CLU.

Aspects of the disclosure relate to methods for generating MS1 type monocytes. In some embodiments, generating MS1 type monocytes comprises incubating CD34+ bone marrow mononuclear cells (BMMCs) in the presence of IL6. In some embodiments, the BMMCs can be hematopoietic stem and progenitor cells (HSPCs). In some embodiments, the CD34+ BMMCs can be derived from bone marrow. In some embodiments, the HSPCs can be derived from cord blood. In some embodiments, the HSPCs can be derived from peripheral blood.

In some embodiments, generating MS1 type monocytes comprises incubating CD34+ bone marrow mononuclear cells (BMMCs) in the presence of IL10. In some embodiments, generating MS1 type monocytes comprises incubating CD34+ bone marrow mononuclear cells (BMMCs) in the presence of IL6 and IL10. In some embodiments, CD34+ BMMCs can be incubated in the presence of plasma from sepsis patients in the presence of IL6, IL10, and IL6/IL10. In some embodiments, CD34+ BMMCs can be incubated in culture media that comprises approximately 20% plasma from sepsis patients. In some embodiments, the CD34+ BMMCs can be incubated in culture media that comprises approximately 20% plasma from sepsis patients in the presence of IL6, IL10, and IL6/IL10 for at least 1, 2, 3, 4, 5, 6, 7, 8, 9, or 10 days. In some embodiments, the CD34+ BMMCs can be incubated in culture media that comprises approximately 20% plasma from sepsis patients in the presence of IL6, IL10, resulting in STAT3-Y705 phosphorylation. In some embodiments, the CD34+ BMMCs as disclosed in the present disclosure can be incubated in the presence of GM-CSF, M-CSF, or both GM-CSF and M-CSF.

In some embodiments, the incubation of the CD34+ BMMCs can result in upregulation of expression of one or more of: S100A8, S100A12, VCAN, RETN, LYZ, MNDA, CTSD, SELL, CYP1B1, CLU, NKG7, MCEMP1, TIMP1, SOD2, CD163, NAMPT, ACSL1, VAMP5, LILRA5, VNN2, ANXA6, CALR, and CTSA compared with CD34+ HSPCs incubated in the presence of plasma from heathy subjects. In some embodiments, the incubation of the CD34+ BMMCs can result in upregulation of expression of S100A8 compared with CD34+ HSPCs incubated in the presence of plasma from heathy subjects. In some embodiments, the incubation of the CD34+ BMMCs can result in upregulation of expression of MNDA compared with CD34+ HSPCs incubated in the presence of plasma from heathy subjects. In some embodiments, the incubation of the CD34+ BMMCs can result in upregulation of expression of VCAN compared with CD34+ HSPCs incubated in the presence of plasma from heathy subjects. In some embodiments, the incubation of the CD34+ BMMCs can result in upregulation of expression of any one of S100A8, MNDA, and VCAN. In some embodiments, the CD34+ BMMCs can be administered to the same subject from whose bone marrow the CD34+ HSPCs were derived.

In some embodiments, the MS1 type monocytes can be used for screening for therapeutics. In some embodiments, the therapeutic can be an inducer of MS1 type monocytes. In some embodiments, the therapeutic can be an inhibitor of MS1 type monocytes. In some embodiments, the incubation of the MS1 type monocytes can delay and/or suppress the proliferation of CD4 T cells. In some embodiments, the incubation of the MS1 type monocytes can delay and/or suppress the proliferation of CD8 T cells. In some embodiments, the incubation of the MS1 type monocytes can delay and/or suppress the proliferation of CD4 T cells and/or the CD8 T cells in the presence of CD3 and CD28. In some embodiments, the incubation of the MS1 type monocytes can result in upregulation of expression of MMP1, PROS1, VCAM1, SST, and FN1. In some embodiments, the incubation of the MS1 type monocytes can result in suppression of inflammatory cytokine gene expression. In some embodiments, the incubation of the MS1 type monocytes can result in suppression of BIRC3 compared with MS1 type monocytes incubated in the presence of sepsis serum. In some embodiments, the incubation of the MS1 type monocytes can result in suppression of CXCL8 compared with MS1 type monocytes incubated in the presence of sepsis serum. In some embodiments, the incubation of the MS1 type monocytes can result in suppression of CSF2 compared with MS1 type monocytes incubated in the presence of sepsis serum. In some embodiments, the incubation of the MS1 type monocytes can result in suppression of CXCL1 compared with MS1 type monocytes incubated in the presence of sepsis serum. In some embodiments, the incubation of the MS1 type monocytes can result in suppression of ID3 compared with MS1 type monocytes incubated in the presence of sepsis serum. In some embodiments, the incubation of the MS1 type monocytes can result in suppression of CCL2 compared with MS1 type monocytes incubated in the presence of sepsis serum. In some embodiments, the incubation of the MS1 type monocytes can result in suppression of NFKBIA compared with MS1 type monocytes incubated in the presence of sepsis serum. In some embodiments, the incubation of the MS1 type monocytes can result in suppression of one or more of: BIRC3, CXCL8, CSF2, CXCL1, ID3, CCL2, and NFKBIA compared with MS1 type monocytes incubated in the presence of sepsis serum.

In some embodiments, the incubation of the MS1 type monocytes comprises incubation with sepsis serum. In some embodiments, the culture media of MS1 type monocytes can result in the suppression of the upregulation of chemokine genes. In some embodiments, the chemokine genes can be associated with cytokine-cytokine receptor interaction. In some embodiments, the chemokine genes can be associated with the NOD-like receptor signaling pathway. In some embodiments, the chemokine genes can be associated with the pathways in cancer. In some embodiments, the chemokine genes can be associated with any one of the cytokine-cytokine receptor interaction, NOD-like receptor signaling pathway, and pathways in cancer. In some embodiments, the MS1 type monocytes can comprise elevated levels of ARG1. In some embodiments, the MS1 type monocytes can comprise elevated levels of iNOS. In some embodiments, the MS1 type monocytes can comprise elevated levels of ROS. In some embodiments, the MS1 type monocytes can comprise elevated levels of any one of ARG1, iNOS, and ROS.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in the present disclosure is for the purpose of description and should not be regarded as limiting. The use of “including,” “comprising,” or “having,” “containing,” “involving,” and variations of thereof in the present disclosure, is meant to encompass the items listed thereafter and equivalents thereof as well as additional items.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. The accompanying drawings are not intended to be drawn to scale. The drawings are illustrative only and are not required for enablement of the disclosure. For purposes of clarity, not every component may be labeled in every drawing. In the drawings:

FIGS. 1A-1F show cohort definition and analysis strategy. FIG. 1A shows the processing pipeline for blood samples used in this study. Total CD45+ peripheral blood mononuclear cells (PBMCs) and enriched dendritic cells for subject groups were labelled with cell hashing antibodies and loaded on a droplet-based scRNA-seq platform. Cells were demultiplexed and multiplets were removed based on calls for each barcoding antibody. FIG. 1B shows a schematic and number of subjects for each cohort profiled in this study. FIG. 1C shows the age distribution of subjects and controls analyzed in this study. FIG. 1D shows time to enrollment from hospital presentation for each subject across all cohorts. Boxes show the mean and interquartile range (IQR) for each cohort, with whiskers extending to 1.5× the IQR in either direction from the top or bottom quartile FIG. 1E shows bar plots showing fractions of Gram-positive and Gram-negative pathogens for each cohort. FIG. 1F shows an analysis pipeline: cell states were identified via two-step clustering and fractional abundances thereof were compared to find sepsis-specific states. Further signatures were derived from these states using differential gene expression and gene module analysis. These signatures were validated in external sepsis datasets via a combination of bulk gene expression deconvolution, direct mapping of gene signatures, and meta-analysis. Experiments were performed to identify surface markers, develop a model system for induction, analyze the epigenomic profile, and characterize the functional phenotype of the identified cell state.

FIGS. 2A-2G show scRNA-seq identifies sepsis-specific immune cell states and gene signatures. FIG. 2A shows t-distributed stochastic neighbor embedding (t-SNE) plots of cells for each cell type (n=32,341, 7,970, 9,390, 58,557 and 14,299 cells for T, B, NK, monocyte (mono) and dendritic (DC) cells, respectively), colored by embedding density of cells from subjects with sepsis (Int-URO, URO, Bac-SEP and ICU-SEP; left) and cell state (right). FIG. 2B shows select marker genes that are differentially expressed (false-discovery rate (FDR)<0.05, two-tailed Wilcoxon rank-sum test) in each cell state, when compared with other cell states within the same cell type. Color scale corresponds to z-scored, log-transformed mean gene expression counts for each cell state. TS, T cell states; BS, B cell states; NS, NK cell states; MS, monocyte states; DS, dendritic cell states; MK, megakaryocytes. FIG. 2C shows fraction of total CD45+ cells across each subject type for total monocytes (left) and MS1 cells (right). In the Control group, points for healthy controls that were follow-up samples from enrolled Leuk-UTI, Int-URO, and URO subjects are indicated as black symbols, and those for matched healthy control samples from an outside source are indicated as aqua symbols. FDR values are shown when comparing each disease state with healthy controls (two-tailed Wilcoxon rank-sum test, corrected for testing of multiple states). Boxes show the median and IQR for each subject cohort, with whiskers extending to 1.5× the IQR in either direction from the top or bottom quartile. Sample size (n) for each cohort is indicated in FIG. 1B. FIG. 2D shows a volcano plot showing results from differential expression analysis (two-sided Wilcoxon rank-sum test) between MS1 cells from ICU-SEP and MS1 cells from ICU-NoSEP subjects. Genes with log 2FC>1 are highlighted in red, and the top 5 genes with the highest positive fold-changes are labeled. Sample sizes were 2,153 and 1,442 cells from the 8 ICU-SEP and 7 ICU-NoSEP subjects, respectively. FIG. 2E shows box and swarm plots showing the mean expression (log₂ UMI counts) of PLAC8 and CLU in MS1 cells for each subject from the ICU-SEP and ICU-NoSEP cohorts. Boxes show the median and IQR for each subject cohort, with whiskers extending to 1.5× the IQR in either direction from the top or bottom quartile. FIGS. 2F-2G show scatterplots showing correlation between mean gene module usage in MS1 cells and sequential organ-failure assessment (SOFA) scores for Int-URO and URO subjects. Line and shadow indicate linear regression fit and 95% confidence interval, respectively.

FIGS. 3A-3I show analysis of the MS1 cell state as a sepsis marker. FIG. 3A shows a receiver-operating characteristic (ROC) curve for subject classification based on (top) MS1 abundance or (bottom) mean PLAC8 and CLU expression in MS1 cells, and gene expression score-based classifiers (FAIM3/PLAC8, SeptiCyte Lab). MS1 is taken as the fraction of total CD45+ cells per subject, as defined by scRNA-seq. Gene-set scores were calculated, as detailed in each corresponding publication, on the pseudo-bulk gene expression matrix obtained by summing read counts from all cells of each subject. SEP indicates all subjects with sepsis analyzed in this study (Int-URO, URO, Bac-SEP, ICU-SEP). FIGS. 3B-3C are forest plots showing the effect size (log 2 (standardized mean difference between indicated patient phenotypes)) of inferred MS1 abundance in each dataset from bulk gene expression deconvolution. Accession numbers of the data from each study are listed on the left. Boxes indicate the effect size in an individual study, with whiskers extending to the 95% confidence interval. Size of the box is proportional to the relative sample size of the study. Diamonds represent the summary effect size among the subject groups, determined by integrating the standardized mean differences across all studies. The width of the diamond corresponds to its 95% confidence interval. FIG. 3D shows individual ROC curves for sepsis versus noninfected healthy controls; analysis includes each study in FIG. 3B for which the number of sepsis subjects and controls were both greater than 5. Sample size (n)=751 total subjects from 9 cohorts. FIG. 3E shows ROC curves for classifying sepsis versus sterile inflammation (n=696 total subjects from 7 cohorts) on the basis of the mean expression of PLAC8, CLU, and the top 6 MS1 marker genes (RETN, CD63, ALOX5AP, SEC61G, TXN, and MT1X). Black curves in FIGS. 3D-3E indicate the summary ROCs. FIG. 3F shows flow cytometry density plots of LIN-CD14+ monocytes (where LIN− cells are those negative for the indicated lineage markers) gated on surface expression of IL1R2 and HLA-DR. Percentage of the population over total CD14+ monocytes in each quadrant is indicated. Each density plot shows peripheral blood mononuclear cells (PBMCs) from a single subject analyzed in one experiment. FIG. 3G shows fractional abundance of CD14+HLA-DR^{lo}IL1R2^{hi} monocytes by flow cytometry in Control, Leuk-UTI, Int-URO and URO subjects (sample size (n)=6, 4 and 6, respectively). Samples used for this analysis were from the primary cohort (Control, Leuk-UTI, Int-URO, URO). Boxes show the median and IQR for each subject cohort, with whiskers extending to 1.5× the IQR in either direction from the top or bottom quartile. FIG. 3H shows correlation of MS1 fractions defined by scRNA-seq (y-axis) and CD14+, HLA-DR^{lo}IL1R2^{hi} monocyte fractions of CD45+ cells (x-axis) from n=4 Leuk-UTI and n=6 URO subjects from FIG. 3G. Significance of the correlation (Pearson r) was calculated with a two-sided permutation test. FIG. 3I shows scRNA-seq of sorted CD14+HLA-DR^{lo}IL1R2^{hi}, monocytes and original MS1 cells visualized with t-SNE projection. Top scatterplot (n=15,021 cells) shows original classification of cells from the cohorts, and the bottom shows scaled embedding density of sorted cells (n=7,098 cells) in the same projection.

FIGS. 4A-4N show induction and characterization of MS1 monocytes. FIG. 4A shows flow cytometry contour plots showing IL1R2 and HLA-DR of cells gated on the CD14+ fraction from either bone marrow (BM, top row) or peripheral blood (PB, bottom row) mononuclear cells. Cells are either freshly thawed (left column) or stimulated with 100 ng mL⁻¹ LPS for 3 days in hematopoietic stem cell (HSC) cytokine-rich medium (right column). Each density plot shows cells from a single donor analyzed in one experiment. FIG. 4B shows fractional abundance of HLA-DR^{lo}IL1R2^{hi} cells among CD14+ monocytes in PB or BM mononuclear cells stimulated with either 100 ng mL⁻¹ LPS (top) or Pam3CSK4 (bottom) over time (0 to 4 days). Different symbols indicate cells obtained from different healthy donors. P values are calculated from a two-sided Wilcoxon rank-sum test between day 0 and day 4. FIGS. 4C-4D show scRNA-seq of BM mononuclear cells (n=8,702 cells) incubated in HSC cytokine-rich medium with no treatment or 100 ng mL⁻¹ LPS or Pam3CSK4 for 4 days. Cells are visualized on a uniform manifold approximation and projection (UMAP) plots and colored by treatment (FIG. 4C) or MS1 score (FIG. 4D). MS1 scores are given as the ratio of the average expression of the top 15 MS1 marker genes to the average expression of a randomly sampled set of 50 reference genes. In each plot, the cluster with the highest MS1 score is circled. Dotted circle indicates monocyte clusters. Inset shows the mean fractional abundance of the iMS1 cluster among monocytes across each donor and treatment condition; each individual point is calculated by randomly sampling the data and clustering the subsampled dataset (e) n=20 iterations). FIG. 4E shows UMAP projections of Pam3CSK4 and LPS stimulated BM myeloid and progenitor cells (HSC/MPP, CMP, GMP, Mono, and iMS1) colored by cell type (top) and diffusion pseudotime (bottom). FIG. 4F shows Principal-component analysis (PCA) plots of assay for transposase-accessible chromatin using sequencing (ATAC-seq) peak accessibility profiles for four different sorted monocyte populations: PB-Mono, PB-MS1, BM-Mono (CD14+ monocytes from freshly thawed BM cells), and BM-iMS1 (CD14+ monocytes from BM cells stimulated for 4 days with 100 ng mL⁻¹ LPS in HSC cytokine-rich medium. Experiments were performed on two donors with two technical replicates each. FIG. 4G is a Venn diagram showing overlap of differentially accessible peaks (FDR<0.1, edgeR exact test) from monocyte populations in PB and BM. FIG. 4H depicts sequence logos showing the top 3 enriched motifs in the differentially accessible peaks when comparing PB-Mono and PB-MS1. Percentages indicate the number of differential peaks that contain the motif for PB and BM (n.d. indicates that motif was not detected in the enrichment analysis). FIG. 4I shows relative expression (normalized log 2 (transcripts per kilobase million (TPM)) of the CEBP family of transcription factors across the four monocyte populations. FIG. 4J shows scaled expression (normalized log counts) of the CEBP family of transcription factors along the pseudotime trajectory in FIG. 4E. FIG. 4K shows top 10 enriched pathways in the differentially accessible genes (FDR<0.1) when PB-MS1 cells are rested for 24 h and subsequently stimulated with 100 ng ml⁻¹ LPS. RNA-seq experiments were performed on 2 donors, with 3 technical replicates each. Sizes of circles are proportional to the number of gene hits in a set, whereas color represents the enrichment score of each gene set. FIG. 4L shows TNF expression and FIG. 4M shows TNFα protein levels in the supernatant of the indicated four sorted monocyte populations after LPS stimulation. P values are calculated from a two-sided Wilcoxon rank-sum test between LPS-stimulated Mono and iMS1 cells. Protein measurements were performed on two donors with two technical replicates each. FIG. 4N is a Venn diagram showing overlap of differentially accessible genes (FDR<0.1, edgeR exact test) from the indicated sorted monocyte populations after LPS stimulation. Top 10 genes with highest significance are indicated in red for the PB-MS1-exclusive set of genes and the overlap between BM-iMS1 and PB-MS1. HSC/MPP, hematopoietic stem cells and multipotent progenitors; CMP, common myeloid progenitors; GMP, granulocyte-macrophage progenitor.

FIGS. 5A-5E depict scRNA-seq demultiplexing and quality assessment. FIG. 5A shows a sample strategy for gating for hashtag oligo (HTO) positive cells based on UMI tag counts of each barcode. FIG. 5B shows a histogram of cells per 10× gel beads in emulsion (GEM) barcode for one representative channel. Data are shown for one channel with n=15,304 detected GEMs. FIG. 5C shows t-distributed stochastic neighbor embedding (t-SNE) plots of all cells (n=126,351 cells total from 65 individuals) in the study colored by institution of origin of the cohort, hashtag barcode, and processing batch. Adjusted Rand index is shown for each when compared with cell state assignments. FIG. 5D shows violin plots (n=126,351 cells total from 65 individuals) of various quality control metrics for the full scRNA-seq dataset generated in this study. FIG. 5E shows violin plots of genes detected across different cell-types (n=32,341, 7,970, 9,390, 58,557, 14,299, 3,794 cells for T, B, NK, Mono, DC, and MK, respectively). Violin plots show a kernel density estimate of the data, using Scott's rule to calculate the appropriate kernel bandwidth. The violin extends to 2× the bandwidth in both directions.

FIGS. 6A-6F show robust identification of cell states with two-step clustering. FIGS. 6A-6B show identification of immune cell types based on marker genes of low-resolution clusters. Color scale in FIG. 6B corresponds to z-scored, log 2-transformed mean gene expression counts across all cells (n=126,351 cells total from 65 individuals). FIGS. 6C-6D show assessment of cluster robustness for FIG. 6C T-cells and FIG. 6D monocytes. Boxplots show distributions of Rand indices when comparing clustering solutions with subsampled data (20 iterations). Boxes show the median and IQR for each resolution, with whiskers extending to 1.5 IQR in either direction from the top or bottom quartile. T-SNE plots show final assigned states for each cell type. FIGS. 6E-6F show barplots showing the fraction of each patient FIG. 6E and batch FIG. 6F in each of the 16 cell states (number of patients or batches with each state is indicated).

FIGS. 7A-7C show flow cytometry abundances of classical myeloid cell states. FIG. 7A shows gating strategy for determination of CD14+ mono, CD16+ mono, and dendritic cell abundance. FIG. 7B shows correlation of fractional abundances defined by flow cytometry and scRNA-seq for each patient (n=65 individuals). FIG. 7C shows fractional abundance of the three cell types based on flow cytometry, grouped by disease state. Sample size (n) for each cohort is indicated in FIG. 1B. Boxes show the median and IQR for each patient cohort, with whiskers extending to 1.5 IQR in either direction from the top or bottom quartile.

FIGS. 8A-8E show differentially expressed genes in immune cell states. Top 10 differentially expressed genes (false discovery rate; FDR<0.05, two-tailed Wilcoxon rank-sum test) for each cell state when compared with other cells within the same cell type. Heatmaps are grouped according to the parent cell type of the different states: FIG. 8A shows T cells, FIG. 8B shows B cells, FIG. 8C shows NK cells, FIG. 8D shows monocytes, and FIG. 8E shows dendritic cells. n=32,341, 7,970, 9,390, 58,557, 14,299, 3,794 cells for T, B, NK, Mono, DC, and MK, respectively. cDC, conventional dendritic cells; pDC, plasmacytoid dendritic cells; AS DC, AXL-SIGLEC6 dendritic cells.

FIGS. 9A-9B show fractional abundance of states defined by scRNA-seq. FIG. 9A shows cell type and state composition for each patient in each cohort. FIG. 9B shows Pearson correlation matrix of cell states across all patients (n=65 patients).

FIGS. 10A-10C show disease-specific abundance of cell types and states. Boxplots showing fractional abundances of cell types, as in FIG. 10A, and states among patients grouped by patient cohort, as in FIG. 10B. False discovery rate (FDR) values are shown when comparing each disease state with healthy controls (two-tailed Wilcoxon rank-sum test, corrected for multiple testing of states). Sample size (n) for each cohort is indicated in FIG. 1B. FIG. 10C shows boxplots showing absolute abundances of cell states among patients (for which leukocyte counts were available), grouped by patient cohort. Boxes show the median and interquartile range (IQR) for each patient cohort, with whiskers extending to 1.5 IQR in either direction from the top or bottom quartile. Sample size n=10, 6, 10, 3, 6, and 4 patients, for Leuk-UTI, Int-URO, URO, BAC-SEP, ICU-SEP and ICU-NoSEP, respectively.

FIGS. 11A-11G show in-depth analysis of the gene expression profile of MS1. FIG. 11A shows top 30 differentially expressed genes (among highly variable genes) when comparing MS1 against other CD14+ monocyte states (MS4 and MS2). FIG. 11B is a dotplot showing enrichment of pathways (KEGG database) for upregulated genes in MS1 vs. MS2 (FDR<0.1, edgeR exact test). Sample size n=15,021 and 11,439 cells for MS1 and MS2, respectively. Sizes of circles are proportional to the number of gene hits in a set, whereas color represents the enrichment score of each gene set. FIG. 11C is a heatmap showing the average expression of genes that are differentially expressed (FDR<0.1, two-sided Wilcoxon rank-sum test) between all MS1 cells from each patient in the ICU-SEP cohort and all MS1 cells from each patient in the ICU-NoSEP cohort (n=2,153 and 1,442 cells from 8 and 7 ICU-SEP and ICU-NoSEP patients, respectively). To specifically identify genes that discriminate the two patient populations, genes are filtered for expression in-group fraction >0.4 and out-group fraction <0.6. FIG. 11D is a k-selection plot to determine the number of components for non-negative matrix factorization (NMF). Dotted line indicates selected number of components for further analysis. FIG. 11E is a t-SNE plot of MS1 cells (n=15,021 cells) colored by patient cohort of origin. FIG. 11F shows scaled TPM usage values of each gene module derived from NMF analysis. The top 20 genes in each module are shown. FIG. 11G depicts scatterplots showing correlation between mean gene module usage in MS1 cells and sequential organ failure assessment (SOFA) scores for Int-URO and URO patients (top row), and Bac-SEP and ICU-SEP patients (bottom row). Sample size (n) for each cohort is indicated in FIG. 1B. Significance of the correlation (Pearson r) was calculated with a two-sided permutation test. Line and shadow indicate linear regression fit and 95% confidence interval, respectively.

FIGS. 12A-12F show state-specific expression of sepsis signature genes. FIG. 12A depicts t-SNE plots showing scaled gene expression counts across all cells (n=126,351 total from 65 individuals) for FAIM3-PLAC8 and SeptiCyte Lab genes (+ or − indicates that a gene is up- or down-regulated, respectively, in sepsis). FIG. 12B shows mean expression of PLAC8 in T cell (top row) and monocyte (bottom row) states across patients grouped by cohort. Sample size (n) for each cohort is indicated in FIG. 1B. FIGS. 12C-12F are heatmaps showing state-specific expression of Sepsis Metascore genes as in FIG. 12C, genes previously associated with sepsis mortality as in FIG. 12D or survival as in FIG. 12E and sepsis-linked GWAS genes as in FIG. 12F. Color scale corresponds to z-scored, log 2-transformed mean gene expression counts for cell state.

FIGS. 13A-13F show state matrix generation and performance comparison of gene-based signatures. FIG. 13A shows optimization of the number of marker genes per cell state in the basis matrix for deconvolution. Mean deconvolution accuracy is shown for pseudo-bulk gene expression data generated for each patient in the study (n=5 patients). Accuracy is measured as high correlation or low root mean-squared error (RMSE) between predicted and true values. The dotted line indicates the number of genes used for downstream analysis. FIG. 13B shows gene expression correlation of all states using the signature matrix with 100 genes per cell state (1,201 total, union of all). FIG. 13C is a scatterplot showing deconvolution accuracy (measured by Pearson correlation between true and inferred fractions) increases with median fractional abundance of cell states. FIG. 13D is summary AUROCs (area under the receiver operating characteristic curve) of the mean expression of PLAC8, CLU, and the indicated number of MS1 marker genes when classifying sepsis patients against sterile inflammation in published datasets. Top and bottom lines indicate the 95% confidence interval of the summary AUROC. The dotted line indicates the number of MS1 marker genes used for downstream analysis. FIGS. 13E-13F show individual ROC curves of FAIM3-PLAC8 Ratio, SeptiCyte Lab, and Sepsis MetaScore on published datasets comparing sepsis vs. healthy controls, as in FIG. 13E (n=751 total patients from 9 cohorts, or sepsis vs. sterile inflammation, as in FIG. 13F (n=696 total patients from 7 cohorts).

FIGS. 14A-14H show scRNA-seq characterization of stimulated bone-marrow mononuclear cells. BM mononuclear cells incubated in HSC cytokine-rich media with no treatment (NT) or 100 ng/mL LPS or Pam3CSK4 (Pam 3) for 4 days. Cells (n=8,702) are visualized on a UMAP projection and colored by treatment as in FIG. 14A, Leiden clusters as in FIG. 14B, and cell-type annotations as in FIG. 14C. FIG. 14D is a matrix plot showing the mean log-transformed UMI counts of the top 5 differentially expressed genes (FDR<0.01, two-tailed Wilcoxon rank-sum test) for each cluster. FIG. 14E is a heatmap showing differentially expressed genes (FDR<0.01, two-tailed Wilcoxon rank-sum test) between clusters 3 (CD14 monocytes, n=786 cells) and 14 (iMS1 cluster, n=130 cells). FIG. 14F depicts UMAP projections of non-stimulated BM myeloid and progenitor cells (HSC/MPP, CMP, GMP, Mono; n=1,976 cells total) colored by cell type (top) and diffusion pseudotime (bottom). FIG. 14G depicts violin plots showing pseudotime values for each cell type in each stimulation condition. Sample size n=1,976 and 901 cells for NT and LPS or Pam3 treatments, respectively. Violin plots show a kernel density estimate of the data, using Scott's rule to calculate the appropriate kernel bandwidth. The violin extends to 2× the bandwidth in both directions. FIG. 14H depicts volcano plots showing differentially expressed genes between LPS or Pam3CSK4 and untreated cells for the HSC/MPP (n=1,168 cells) and GMP populations (n=519 cells). Differentially expressed genes (log FC>0.3, p<0.05; two-sided Wilcoxon rank-sum test) are shown in red. Known receptors (based on a previously published database) that are differentially expressed are labelled. Abbreviations: HSC/MPP, hematopoietic stem cells and multipotent progenitors; CMP, common myeloid progenitors; GMP, granulocyte-macrophage progenitor; MEP, megakaryocyte-erythroid progenitors; MYL, myeloblasts; RBC, red blood cells.

FIGS. 15A-15M show characterization of the gene expression module of MS1 cells incubated with HSPCs. FIG. 15A is a scatterplot showing comparisons of the cell states versus the gene expression module usage of MS1, MS2, MS3, and MS4 by conducting non-negative matrix factorization to characterize the gene expression module of MS1. FIG. 15B is a network diagram of the MS1 gene module after conducting non-negative matrix factorization. FIG. 15C is a graph showing that the MS1 module usage correlated with IL10 level. FIG. 15D is a graph showing that the MS1 module usage correlated with IL6 level. FIG. 15E is a graph showing that the incubation of CD34+ hematopoietic stem & progenitor cells (HSPCs) in sepsis plasma produced monocytes with higher expression of MS1 genes compared to healthy plasma. FIG. 15F is a graph showing that the trajectory analysis showed differentiation pathways from hematopoietic stem & progenitor cells (HSPCs) to MS1-like monocytes. HSPCs were incubated in 20% sepsis plasma for 7 days. FIG. 15G is a heatmap graph showing that the incubation in sepsis plasma of hematopoietic stem & progenitor cells (HSPCs) with IL6 or IL10 receptors knocked out showed reduction in expression of MS1 genes. FIG. 15H is a graph showing that the incubation in sepsis plasma of hematopoietic stem & progenitor cells (HSPCs) with IL6 or IL10 receptors knocked out showed partial rescue of HLA-DR expression. FIG. 15I is a graph showing that the incubation of hematopoietic stem & progenitor cells (HSPCs) in sepsis plasma with neutralizing antibodies to IL6 and IL10 showed partial rescue of HLA-DR expression. FIG. 15J is a graph showing that the incubation of hematopoietic stem & progenitor cells (HSPCs) in sepsis plasma resulted in STAT3-Y705 phosphorylation, representing downstream targets of both IL6 and IL10 signaling. FIG. 15K is a heatmap graph showing that the incubation of CD34+ hematopoietic stem & progenitor cells (HSPCs) in IL6, IL10 or IL6 and IL10 in the presence or absence of GM-CSF resulted in the up-regulation of MS1 genes. FIG. 15L is a graph showing a comparison of the MS1 module derived de novo from CD34+ hematopoietic stem & progenitor cells (HSPCs) differentiated with cytokines versus those from patient PBMCs. FIG. 15M is a graph showing the usage of the MS1 module derived de novo from CD34+ hematopoietic stem & progenitor cells (HSPCs) differentiated across different cytokine conditions: (1) NT: no cytokine, (2) IL6 only, (3) IL10 only, and (4) IL6 and IL10 in the presence or absence of GM-CSF and M-CSF.

FIG. 16A-16E show gene expression of MS1 module derived from CD34+ HSPCs co-incubated with various cytokine conditions. FIG. 16A is a heatmap graph showing the analysis of genes along the trajectory in FIG. 15F that show different dynamic patterns of the MS1 genes. Among the MS1 genes shown in the heatmap graph, S100A8, MNDA, and VCAN gene expression was up-regulated after 24 hour incubation and remained up-regulated throughout the tested time points. FIG. 16B is a graph showing that short term stimulation (24 h) of hematopoietic stem & progenitor cells (HSPCs) with sepsis plasma resulted in up-regulation of the S100A8, MNDA, and VCAM genes that were up-regulated early as shown in FIG. 16A. FIG. 16C is a graph showing that short term stimulation (24 h) of CD34+hematopoietic stem & progenitor cells (HSPCs) with cytokines in various concentrations resulted in up-regulation of S100A8. The cytokine conditions were: (1) NT: no treatment, (2) IL6-1, (3) IL6-10, (4) IL6-100, (5) IL10-1, (6) IL10-10, (7) IL10-100, (8) HC, and (9) sepsis plasma. FIG. 16D is a graph showing that short term stimulation (24 h) of CD34+ hematopoietic stem & progenitor cells (HSPCs) with cytokines in various concentrations resulted in up-regulation of MNDA. The cytokine conditions were: (1) NT: no treatment, (2) IL6-1, (3) IL6-10, (4) IL6-100, (5) IL10-1, (6) IL10-10, (7) IL10-100, (8) HC, and (9) sepsis plasma. FIG. 16E is a graph showing that short term stimulation (24 h) of CD34+ hematopoietic stem & progenitor cells (HSPCs) with cytokines in various concentrations resulted in up-regulation of VCAN. The cytokine conditions were: (1) NT: no treatment (2) IL6-1, (3) IL6-10, (4) IL6-100, (5) IL10-1, (6) IL10-10, (7) IL10-100, (8) HC, and (9) sepsis plasma.

FIG. 17 shows co-incubation of iMS1 cells with activated CD4 T cells and CD8 T cells delayed and/or suppressed the proliferation of the respective T cells. CD4 T cells and CD8 T cells were incubated with the following treatments: (1) negative control without the presence of CD3/CD28, (2) positive control with the presence of CD3/CD28, (3) CD3/CD28+iMS1 cells, and (4) CD3/CD28+iMono cells. The CD4 T cell and the CD8 T cells were derived from a different donor than the donor of the iMS1 cells.

FIG. 18 is a heatmap of differential gene expression of renal epithelial cells co-incubated with iMS1 cells versus iMono cells. Genes that were upregulated by the iMS1 cells included MMP1, PROS1, VCAM1, SST, and FN1.

FIG. 19 is a heatmap of differential inflammatory cytokine gene expression of renal epithelial cells with the addition of the following treatments: (1) healthy serum, (2) sepsis serum only, (3) sepsis serum+iMono cells, or (4) sepsis serum+iMS1 cells.

FIG. 20A and FIG. 20B show the expression of various chemokine genes in the endothelial cells incubation with conditioned media from MS1 cells. FIG. 20A is a volcano plot showing results from differential expression analysis results (two-sided Wilcoxon rank-sum test). Chemokine genes are suppressed with the presence of MS1 cells. FIG. 20B is a dotplot showing enrichment of pathways associated with the downregulated chemokine gene expression in MS1 cells versus MS2 cells. Sizes of circles are proportional to the number of gene hits in a set, whereas color represents the enrichment score of each gene set.

FIG. 21A and FIG. 21B show the phenotype of the MS1 cells (iMS1). FIG. 21A shows graphs of the levels of reactive oxygen species (ROS) by detecting MitoSOX-Red or Mito Tracker Green in MS1 cells (iMS1) versus iMono cells. FIG. 21B shows the % ARG1^{hi} (arginase) and the % iNOS^{hi} (nitric oxide synthase) with no treatment (NT), LPS or Pam3CSK4 (Pam 3) in MS1 cells (iMS1) versus iMono cells.

### DETAILED DESCRIPTION OF THE INVENTION

Aspects of the present disclosure relate to methods for measuring an immune cell signature in a subject having, suspected of having, or at risk for sepsis. Such methods may be useful for clinical purposes, such as for identifying a subject having a bacterial infection and/or sepsis, selecting a treatment for a bacterial infection and/or sepsis, monitoring progression of a bacterial infection and/or sepsis (e.g., progression of a bacterial infection to sepsis), assessing the efficacy of a treatment against a bacterial infection and/or sepsis, or determining a course of treatment for a subject having, suspected of having, or at risk for, a bacterial infection and/or sepsis. Methods described in the present disclosure may also be useful for non-clinical applications, such as research purposes, including, e.g., studying the mechanism of sepsis development and/or biological processes and/or immune responses involved in sepsis, and developing new therapies for bacterial infections and/or sepsis based on such studies.

### Immune Cell Signatures

Methods described herein are based, at least in part, on the identification of an immune cell signature in subjects having, suspected of having, or at risk for, sepsis. As used in the present disclosure, “an immune cell signature” in a subject having, suspected of having, or at risk for, sepsis refers to a distinguishing feature of immune cells in a subject having, suspected of having, or at risk for, sepsis compared to a control. The immune cell signature can correspond to a fraction, portion, or subpopulation of immune cells that is elevated or reduced in subjects having sepsis compared to control subjects.

Sepsis or septicemia can occur when chemicals released in the bloodstream to fight an infection trigger inflammation throughout the body. Sepsis can cause a cascade of changes that damage multiple organ systems, leading them to fail, sometimes resulting in death.

The present disclosure encompasses any type of immune cell. Examples of immune cells include, but are not limited to, leukocytes, monocytes, dendritic cells, B cells, T cells, and NK cells. A marker of an immune cell (e.g., a cell surface marker) can encompass any gene or protein for which expression or absence of expression can be used to identify or can contribute to identifying or classifying the immune cell. Examples of a marker of an immune cell include, but are not limited to, CD14, CD16, CD64, CD192, HLA-DR, CD195, TNFR1, TNFR2, CX3CR1, CD3, CD19, CD45, CD11c, CD56, CD94, and NKp46.

Immune cells can be identified based on the presence, absence, or level of a marker (e.g., a cell surface marker such as CD45). For example, monocytes expressing the CD45 marker may be referred to as CD45+ monocytes. Subpopulations of CD45+ monocytes may be further identified based on the presence, absence, or level of other markers, such as IL1R2, HLA-DR, and CD14. Aspects of the present disclosure relate to an immune cell signature for sepsis comprising elevated levels of CD45+ monocytes that are IL1R2^{hi}, HLA-DR^{lo}, and CD14+ relative to a control.

A variety of immune cell signatures may be present in a population of immune cells. For example, a population of CD14+ monocytes may comprise a fraction of CD14+ monocytes characterized by high expression of RETN, IL1R2, and CLU, and a fraction of CD14+ monocytes characterized by high expression levels of class II MHC. In some embodiments, a population of immune cells (e.g., a population of CD14+ monocytes) comprises at least one fraction characterized by high expression of RETN, IL1R2, and CLU relative to a control.

In some embodiments, the fraction of immune cells comprises CD14+ monocytes expressing elevated levels of RETN, IL1R2, and CLU compared to a control population of CD14+ monocytes. In some embodiments, the fraction of immune cells comprises CD14+ monocytes expressing elevated levels of class II MHC genes compared to a control population of CD14+ monocytes. In some embodiments, the fraction of immune cells comprises CD14+ monocytes expressing CD16. In some embodiments, the fraction of immune cells comprises CD14+ monocytes expressing reduced levels of class II MHC and inflammatory cytokines compared to a control population of CD14+ monocytes.

In some embodiments, a subject has elevated levels of an immune cell signature (e.g., CD45+ monocytes that are IL1R2^{hi}, HLA-DR^{lo}, and CD14+) relative to a control. In some embodiments, “elevated levels” refers to levels that are at least 5%, at least 10%, at least 20%, at least 30%, at least 40%, at least 50%, at least 60%, at least 70%, at least 80%, at least 90%, at least 100%, or at least 2-fold, at least 5-fold, at least 10-fold, at least 20-fold, at least 50-fold, or at least 100-fold elevated relative to a control.

In some embodiments, a subject has reduced levels of an immune cell signature relative to a control. In some embodiments, “reduced levels” refers to levels that are at least 5%, at least 10%, at least 20%, at least 30%, at least 40%, at least 50%, at least 60%, at least 70%, at least 80%, at least 90%, at least 100%, or at least 2-fold, at least 5-fold, at least 10-fold, at least 20-fold, at least 50-fold, or at least 100-fold reduced relative to a control.

In some embodiments, one or more genes may be differentially expressed in a fraction of immune cells from a subject having sepsis relative to a control. For example, expression of a gene may be elevated or reduced in a subject having sepsis relative to a control. Examples of genes that may be differentially expressed in a fraction of immune cells from a subject having sepsis relative to a control include, but are not limited to, RETN, CLU, IL1R2, MS4A6A, HLA-DRA, HLA-DRB1, FCGR3A, MS4A7, FTH1, C1orf56, CYBB, and CTNNB1. In some embodiments, genes described in the present disclosure may have an expression level in a fraction of immune cells from a subject having sepsis that deviates (e.g., is enhanced or reduced) from a control by at least 5%, at least 10%, at least 20%, at least 30%, at least 40%, at least 50%, at least 60%, at least 70%, at least 80%, at least 90%, at least 100%, or at least 2-fold, at least 5-fold, at least 10-fold, at least 20-fold, at least 50-fold, or at least 100-fold.

In some embodiments, the level of at least one of RETN, CLU, IL1R2, MS4A6A, MS4A7, FTH1, and CYBB is elevated in a subject having sepsis relative to a control. In some embodiments, the level of at least one of HLA-DRA, HLA-DRB1, and CYBB is reduced in a subject having sepsis relative to a control.

Methods for Generating MS1 Type Monocytes from Bone Marrow Cells

Aspects of the present disclosure relate to methods for generating and producing MS1 type monocytes. In the present methods, CD34+ bone marrow mononuclear cells (BMMCs) can be used in the presence of IL6, IL10, or both IL6 and IL10. As known the art, BMMCs can represent a variety of cell types. Without wishing to be bound by any theory, BMMCs are a mixed population of single nucleus cells including monocytes, lymphocytes, and hematopoietic stem and progenitor cells, which have a single round nucleus, and are isolated from whole bone marrow aspirate by density gradient. For example, BMMC as disclosed in the present disclosure can be hematopoietic stem and progenitor cells (HSPC). HSPC transplantations may require prior harvesting of allogeneic or autologous HSPCs. HSPCs are usually present in bone marrow during the entire life, in cord blood (CB) at birth, or in peripheral blood (PB) under particular circumstances. HSPCs were first harvested in BM and later in CB and PB. In some embodiments, HSPCs can be derived from any suitable source. The disclosure of HSPCs and their source are disclosed in Hequet, “Hematopoietic Stem and Progenitor Cell Harvesting: Technical Advances and Clinical Utility, Journal of Blood Medicine 2015:6 55-67, which is incorporated by reference herein in its entirety.

In some embodiments, the CD34+ bone marrow mononuclear cells (BMMCs) are incubated in the presence of plasma from sepsis patients for at least 1, 2, 3, 4, 5, 6, 7, 8, 9, or 10 days. In some embodiments, the CD34+ bone marrow mononuclear cells (BMMCs) are incubated in the presence of plasma from sepsis patients in the presence of IL6. In some embodiments, the CD34+ bone marrow mononuclear cells (BMMCs) are incubated in the presence of plasma from sepsis patients in the presence of IL10. In some embodiments, the CD34+ bone marrow mononuclear cells (BMMCs) are incubated in the presence of plasma from sepsis patients in the presence of IL6 and IL10. In some embodiments, the CD34+ bone marrow mononuclear cells (BMMCs) are incubated in the presence of plasma from sepsis patient in the presence of GM-CSF. In some embodiments, the CD34+ bone marrow mononuclear cells (BMMCs) are incubated in the presence of plasma from sepsis patient in the presence of M-CSF. In some embodiments, the CD34+ bone marrow mononuclear cells (BMMCs) are incubated in the presence of plasma from sepsis patient in the presence of GM-CSF and M-CSF. In some embodiments, the CD34+ bone marrow mononuclear cells (BMMCs) are incubated in the presence of plasma from sepsis patient in the presence of one or more cytokines. In some embodiments, incubation of the CD34+ bone marrow mononuclear cells (BMMCs) in the presence of plasma from sepsis patients can result in STAT3-Y705 phosphorylation. In some embodiments, the MS1 type monocytes as disclosed in the present disclosure can induce immunosuppression. In some embodiments, the MS1 type monocytes as disclosed in the present disclosure can regulate immune functions.

In some embodiments, the CD34+ HSPCs can be administered to a subject following incubation as disclosed in the present disclosure. In some embodiments, the subject can be a patient with hyperactivated immune responses. In some embodiments, the subject is a subject with autoimmunity. In some embodiments, the subject is a subject with infectious immunity with a cytokine storm. In some embodiments, the subject is a subject with transplant rejection. In some embodiments, the subject is a subject with sepsis.

### Measuring Immune Cell Signatures

Aspects of the present disclosure relate to methods for measuring fractions or subpopulations of immune cells. For example, methods may involve measuring the fraction of CD45+ monocytes that are IL1R2^{hi}, HLA-DR^{lo}, and CD14+ in a sample, such as a blood sample, from a subject, and comparing the fraction of CD45+ monocytes that are IL1R2^{hi}, HLA-DR^{lo}, and CD14+ in the sample from the subject to a control. In some embodiments, a subject has or is at risk for bacterial sepsis. In some embodiments, the control is a sample from a healthy subject, such as a subject who does not have or is not at risk for bacterial sepsis.

The present disclosure encompasses measuring any type of immune cell to obtain information related to any number of fractions of immune cells. In some embodiments, methods comprise measuring at least 1 fraction (e.g., a subpopulation of CD14+ monocytes characterized by high expression of RETN, IL1R2, and CLU) of immune cells in a population of immune cells (e.g., a population of CD14+ monocytes). In some embodiments, methods comprise measuring at least 1, at least 2, at least 3, at least 4, at least 5, at least 6, at least 7, at least 8, at least 9, or at least 10 or more fractions of immune cells in a population of immune cells.

In some embodiments, measuring the fraction of immune cells comprises measuring the expression level of certain genes in the fraction of immune cells (e.g., the level of RETN, IL1R2, and/or CLU in CD14+ monocytes). In some embodiments, methods comprise measuring the level of at least 1 gene in the fraction of immune cells. In some embodiments, methods comprise measuring the level of at least 1, at least 2, at least 3, at least 4, at least 5, at least 6, at least 7, at least 8, at least 9, or at least 10 gene in the fraction of immune cells.

Any of the samples described in the present disclosure can be subject to analysis using the methods described in the present disclosure, which involve measuring the fraction of immune cells having certain cellular markers (e.g., the fraction of CD45+ monocytes that are IL1R2^{hi}, HLA-DR^{lo}, and CD14+) and/or the level of certain markers in immune cells (e.g., levels of RETN, IL1R2, and/or CLU in CD14+ monocytes). The fraction of monocytes and/or the expression level of genes described in the present disclosure can be assessed using methods known in the art or those described in the present disclosure.

As used in the present disclosure, the terms “measuring” or “measurement,” or alternatively “detecting” or “detection,” means assessing the presence, absence, quantity, or amount (which can be an effective amount) of a substance within a sample, including the derivation of qualitative or quantitative concentration levels of such substances, or otherwise evaluating the values or categorization of a subject.

The fraction of immune cells (e.g., the fraction of CD45+ monocytes that are IL1R2^{hi}, HLA-DR^{lo}, and CD14+) and/or the expression levels of an immune cell marker may be measured using an immunoassay. Examples of immunoassays include, without limitation, immunoblotting assays (e.g., Western blot), immunohistochemical analysis, flow cytometry assays, immunofluorescence (IF) assays, enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for measuring the fraction of immune cells and/or the expression levels provided in the present disclosure will be apparent to those of skill in the art.

Such immunoassays may involve the use of an agent (e.g., an antibody) specific to the target biomarker, e.g., CD14 or CD45. An agent such as an antibody that “specifically binds” to a target biomarker is a term well understood in the art, and methods to determine such specific binding are also well known in the art. An antibody is said to exhibit “specific binding” if it reacts or associates more frequently, more rapidly, with greater duration and/or with greater affinity with a particular target biomarker than it does with alternative biomarkers. It is also that, for example, an antibody that specifically binds to a first target peptide may or may not specifically or preferentially bind to a second target peptide. As such, “specific binding” or “preferential binding” does not necessarily require (although it can include) exclusive binding. Generally, but not necessarily, reference to binding means preferential binding. In some examples, an antibody that “specifically binds” to a target peptide or an epitope thereof may not bind to other peptides or other epitopes in the same antigen.

As used in the present disclosure, the term “antibody” refers to a protein that includes at least one immunoglobulin variable domain or immunoglobulin variable domain sequence. For example, an antibody can include a heavy (H) chain variable region (abbreviated in the present disclosure as V_{H}), and a light (L) chain variable region (abbreviated in the present disclosure as V_{L}). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26(3):629-39.)) as well as complete antibodies. An antibody can have the structural features of IgA, IgG, IgE, IgD, IgM (as well as subtypes thereof). Antibodies may be from any source, but primate (human and non-human primate) and primatized (e.g., humanized) are preferred.

In some embodiments, a method described in the present disclosure is applied to measure the fraction of immune cells having certain cellular markers in a sample, such as a blood sample, from a subject. In some embodiments, a method described in the present disclosure is applied to measure the fraction of CD45+ monocytes that are IL1R2^{hi}, HLA-DR^{lo}, and CD14+ in a sample, such as a blood sample, from a subject. Such cells may be collected according to routine practice and the fraction of immune cells may be assessed using a method known in the art.

In some embodiments, a method described in the present disclosure is applied to measure the level of certain markers in immune cells in a sample, such as a blood sample, from a subject. In some embodiments, a method described in the present disclosure is applied to measure the level of RETN, IL1R2, and/or CLU in CD14+ monocytes in a sample, such as a blood sample, from a subject. Such cells may be collected according to routine practice and the level of certain markers in immune cells may be assessed using a method known in the art.

It will be apparent to those of skill in the art that this disclosure is not limited to immunoassays. Detection assays that are not based on an antibody, such as mass spectrometry, are also useful for measuring the fraction of immune cells having certain markers and/or the level of certain markers in immune cells as provided in the present disclosure. Assays that rely on a chromogenic substrates can also be useful for measuring the fraction of immune cells having certain markers and/or the level of certain markers in immune cells as provided in the present disclosure.

Alternatively, nucleic acids in a sample can be measured using a method known in the art to obtain information related to the fraction of immune cells having certain markers and/or the level of certain markers in immune cells. In some embodiments, measuring the fraction and/or the level comprises measuring nucleic acid (e.g., DNA or RNA). In some embodiments, measuring nucleic acid comprises a real-time reverse transcriptase (RT) Q-PCR assay or a nucleic acid microarray assay. Methods for measuring nucleic acids include, but are not limited to, polymerase chain reaction (PCR), reverse transcriptase-PCR (RT-PCR), in situ PCR, quantitative PCR (Q-PCR), real-time quantitative PCR (RT Q-PCR), in situ hybridization, Southern blot, Northern blot, sequence analysis, microarray analysis, detection of a reporter gene, or other DNA/RNA hybridization platforms.

Any binding agent that specifically binds to a desired biomarker may be used in the methods and kits described in the present disclosure to measure the level of a biomarker in a sample. In some embodiments, the binding agent is an antibody or an aptamer that specifically binds to a desired protein biomarker. In other embodiments, the binding agent may be one or more oligonucleotides complementary to a coding nucleic acid or a portion thereof. In some embodiments, a sample may be contacted, simultaneously or sequentially, with more than one binding agent that bind different protein biomarkers (e.g., multiplexed analysis).

To measure the fraction of immune cells having a certain marker, a sample can be in contact with a binding agent under suitable conditions. In general, the term “contact” refers to an exposure of the binding agent with the sample or cells collected therefrom for a period of time sufficient for the formation of complexes between the binding agent and the target biomarker in the sample, if any. In some embodiments, the contacting is performed by capillary action in which a sample is moved across a surface of the support membrane.

In some embodiments, the assays may be performed on low-throughput platforms, including single assay format. For example, a low throughput platform may be used to measure the fraction of CD45+ monocytes that are IL1R2^{hi}, HLA-DR^{lo}, and CD14+ in samples (e.g., blood samples) for diagnostic methods, monitoring of bacterial infection and/or treatment progression, and/or predicting whether a bacterial infection may benefit from a particular treatment.

In some embodiments, it may be necessary to immobilize a binding agent to a support member. Methods for immobilizing a binding agent will depend on factors such as the nature of the binding agent and the material of the support member and may require particular buffers. Such methods will be evident to one of ordinary skill in the art.

The type of detection assay used for the detection and/or quantification of immune cell signatures such as those provided in the present disclosure will depend on the particular situation in which the assay is to be used (e.g., clinical or research applications), and on the kind and number of immune cell signatures to be detected, and on the kind and number of patient samples to be run in parallel, among other parameters familiar to one of ordinary skill in the art.

The assay methods described in the present disclosure may be used for both clinical and non-clinical purposes.

### Samples and Subjects

Any of the immune cell signatures described in the present disclosure (e.g., the fraction of CD45+ monocytes that are IL1R2^{hi}, HLA-DR^{lo}, and CD14+), either alone or in combination, can be used in the methods also described in the present disclosure for analyzing a sample from a subject, such as a subject that has or is at risk for sepsis. Results obtained from such methods can be used in either clinical applications or non-clinical applications, including, but not limited to, those described in the present disclosure.

Any sample that may contain immune cells (e.g., a blood sample) can be analyzed by the assay methods described in the present disclosure. In some embodiments, methods described in the present disclosure involve obtaining a sample from a subject. As used in the present disclosure, a “sample” refers to a composition that comprises blood, plasma, protein and/or immune cells, from a subject. A sample includes both an initial unprocessed sample taken from a subject as well as subsequently processed, e.g., partially purified or preserved forms. In some embodiments, the sample is selected from the group consisting of a blood sample, a serum sample, and a plasma sample.

In some embodiments, the sample is enriched for certain immune cells. In some embodiments, the sample comprises peripheral blood mononuclear cells (PBMCs). In some embodiments, the sample comprises CD45+ PMBCs. In some embodiments, the sample comprises lymphocytes (e.g., T cells, B cells, NK cells) and/or monocytes. In some embodiments, the sample comprises CD45+ monocytes. In some embodiments, the sample comprises enriched dendritic cells. In some embodiments, the sample comprises CD45+ monocytes and enriched dendritic cells.

A sample (e.g., a blood sample) can be obtained from a subject using any means known in the art. In some embodiments, the sample is obtained from the subject by removing the sample from the subject. In some embodiments, the sample is obtained from the subject by removing venous blood. In some embodiments, the sample is obtained from the subject by removing arterial blood. In some embodiments, the sample is obtained from the subject by removing capillary blood.

In some embodiments, multiple (e.g., at least 2, 3, 4, 5, or more) samples may be collected from a subject, over time or at particular time intervals, for example, to assess the disease progression or evaluate the efficacy of a treatment.

In certain embodiments, the subject is an animal. In certain embodiments, the subject is a human. In other embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

In some embodiments, a subject is suspected of or is at risk for sepsis. Such a subject may exhibit one or more symptoms associated with sepsis (e.g., fever, low blood pressure, rapid breathing and/or heart rate). Alternatively or in addition, such a subject may have one or more risk factors for sepsis, for example, a bacterial infection. Alternatively, the subject may be a patient having sepsis. Such a subject may have a bacterial infection. In some examples, the subject is a human patient who may be on a treatment of the bacterial infection, for example, an antibiotic. In other instances, such a human patient may be free of such a treatment.

In some embodiments, the subject is a human patient having, suspected of having, or at risk for a bacterial infection. In some embodiments, the bacterial infection is associated with a bacteria selected from the group consisting of Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria; Staphylococcus; Streptococcus; and Salmonella.

In some embodiments, the subject is a human patient having, suspected of having, or at risk for bacterial sepsis. In some embodiments, the bacterial sepsis is associated with a bacteria selected from the group consisting of Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria; Staphylococcus; Streptococcus; and Salmonella.

### Clinical and Non-Clinical Applications

Immune cell signatures described in the present disclosure can be used for various clinical purposes, such as for identifying a subject having, suspected of having, or at risk for sepsis, monitoring the progress of a bacterial infection, assessing the efficacy of a treatment for sepsis, identifying patients suitable for a particular treatment, and/or predicting sepsis in a subject. Accordingly, described in the present disclosure are diagnostic and prognostic methods for sepsis based on an immune cell signature, for example, the fraction of CD45+ monocytes that are IL1R2^{hi}, HLA-DR^{lo}, and CD14+ and/or the level of RETN, IL1R2, and/or CLU in CD14+ monocytes.

When needed, the fraction and/or the level as described in the present disclosure may be normalized with an internal control in the same sample or with a standard sample (having a predetermined amount) to obtain a normalized value. Either the raw value or the normalized value can then be compared with that in a reference sample or a control sample. An elevated value of the fraction and/or the level in a sample obtained from a subject as relative to the value of the same fraction and/or level in the reference or control sample is indicative of sepsis. In some embodiments, an elevated fraction and/or level of an immune signature in a subject indicates that the subject may have sepsis.

In some embodiments, the fraction and/or the level of an immune signature in a sample obtained from a subject can be compared to a predetermined threshold for that fraction and/or level, an elevation from which may indicate the subject may have sepsis.

The control sample or reference sample may be a sample obtained from a healthy individual. Alternatively, the control sample or reference sample may contain a known amount of the fraction and/or the level to be assessed. In some embodiments, the control sample or reference samples is a sample obtained from a control subject.

As used in the present disclosure, a control subject may be a healthy individual, e.g., an individual that is apparently free of a bacterial infection and/or sepsis. A control subject may also represent a population of healthy subjects, who preferably would have matched features (e.g., age, gender, ethnic group) as the subject being analyzed by a method described in the present disclosure.

The control level can be a predetermined level or threshold. Such a predetermined level can represent the fraction and/or the level in a population of subjects that do not have or are not at risk for sepsis (e.g., the average fraction and/or the average level in the population of healthy subjects). It can also represent the fraction and/or level in a population of subjects that have the target disease.

The predetermined level can take a variety of forms. For example, it can be single cut-off value, such as a median or mean. In some embodiments, such a predetermined level can be established based upon comparative groups, such as where one defined group is known to have a sepsis and another defined group is known to not have sepsis. Alternatively, the predetermined level can be a range, for example, a range representing the fraction and/or the levels in a control population.

The control level as described in the present disclosure can be determined by any technology known in the art. In some examples, the control level can be obtained by performing a conventional method (e.g., the same assay for obtaining the fraction and/or the level in a test sample as described in the present disclosure) on a control sample as also described in the present disclosure. In other examples, the fraction and/or the level can be obtained from members of a control population and the results can be analyzed to obtain the control level (a predetermined value) that represents the fraction and/or the level in the control population.

By comparing the fraction and/or the level in a sample obtained from a candidate subject to the reference value as described in the present disclosure, it can be determined as to whether the candidate subject has or is at risk for sepsis. For example, if the fraction and/or the level in a sample of the candidate subject is increased as compared to the reference value, the candidate subject might be identified as having or at risk for sepsis. When the reference value represents the value range of the fraction and/or the level in a population of subjects having sepsis, the value of the fraction and/or the level in a sample of a candidate falling in the range may indicate that the subject has or is at risk for sepsis.

As used in the present disclosure, “an elevated level” or “a level above a reference value” means that the level of an immune cell population (e.g., CD45+ monocytes that are IL1R2^{hi}, HLA-DR^{lo}, and CD14+) is higher than a reference value, such as a pre-determined threshold of a level of the same immune cell population in a control sample. Control levels are described in detail in the present disclosure. An elevated level of an immune cell population can include a level that is, for example, 1%, 5%, 10%, 20%, 30%, 40%, 50%, 60%, 70%, 80%, 90%, 100%, 150%, 200%, 300%, 400%, 500% or more above a reference value. In some embodiments, the level of the immune cell population in a test sample is at least 1.1, 1.2, 1.3, 1.4, 15, 1.6, 1.7, 1.8, 1.9, 2, 2.5, 3, 3.5, 4, 4.5, 5, 5, 6, 7, 8, 9, 10, 50, 100, 150, 200, 300, 400, 500, 1000, 10000-fold or 5 more higher than the level of the immune cell population in a control.

In some embodiments, the candidate subject is a human patient having a symptom of a sepsis. For example, the subject has fever, chills, rapid heart rate, fast breathing or shortness of breath, confusion and/or disorientation, altered level of consciousness, delirium, dizziness, fatigue, flushing, low body temperature, shivering, pain, sweaty skin, low blood pressure, insufficient urine production, organ dysfunction, skin discoloration, sleepiness, or a combination thereof. In other embodiments, the subject has no symptom of sepsis at the time the sample is collected, has no history of a symptom of sepsis, or no history of sepsis.

A subject identified in the methods described in the present disclosure as carrying a sepsis-associated immune cell signature or having sepsis may be subject to a suitable treatment, such as treatment with an antibiotic, as described in the present disclosure. Without wishing to be bound by any theory, treatments for a subject identified as carrying a sepsis-associated immune cell signature or having sepsis may include, but are not limited to intravenous fluids, mechanical ventilation, hospitalization, fluid replacement, IV fluids, vasoconstrictor, blood pressure support, steroid, and central venous catheter. Other treatments are as described in the present disclosure or as known in the art.

Methods and kits described in the present disclosure also can be applied for evaluation of the efficacy of a treatment for sepsis, such as those described in the present disclosure, given the correlation between the level of immune cell signatures disclosed in the present disclosure and sepsis. For example, multiple biological samples (e.g., blood samples) can be collected from a subject to whom a treatment is performed either before and after the treatment or during the course of the treatment. The levels of sepsis-associated immune cell signatures can be measured by any of the assay methods as described in the present disclosure, and values (e.g., amounts) of the sepsis-associated immune cell signatures can be determined accordingly. For example, if an elevated level of a sepsis-associated immune cell signature indicates that a subject has sepsis, and the level of the sepsis-associated immune cell signature decreases after the treatment or over the course of the treatment (e.g., the level of the sepsis-associated immune cell signature is lower in a later-collected sample as compared to that in an earlier-collected sample), this may indicate that the treatment is effective. In some embodiments, the treatment involves an effective amount of a therapeutic agent, such as an antibiotic.

If a subject is identified as not responsive to a treatment, a higher dose and/or frequency of dosage of the therapeutic agent can be administered to the subject. In some embodiments, the dosage or frequency of dosage of the therapeutic agent is maintained, lowered, or ceased in a subject identified as responsive to the treatment or not in need of further treatment. Alternatively, a different treatment can be applied to the subject who is found as not responsive to the first treatment.

In some embodiments, the presence or amount of a sepsis-associated immune cell signature can be used to identify a subject who has sepsis and/or a subject who may be in need of treatment with, for example, an antibiotic. The level of a sepsis-associated immune cell signature in a sample collected from a subject (e.g., a blood sample) having a bacterial infection can be measured by a suitable method, e.g., those described in the present disclosure. If the level of the sepsis-associated immune cell signature is elevated compared to a control, it may indicate that an antibiotic should be administered to the subject. Accordingly, methods disclosed in the present disclosure can further comprise administering an effective amount of an antibiotic to a subject.

Also within the scope of the present disclosure are methods of evaluating the severity of a bacterial infection. For example, as described in the present disclosure, a subject may have a bacterial infection during which the subject does not experience symptoms of sepsis. In some embodiments, the level of a sepsis-associated immune cell signature is indicative of whether the subject will experience, or is experiencing, sepsis.

### Treatment of Sepsis

A subject having or at risk for sepsis, as identified using the methods described in the present disclosure, may be treated with any appropriate anti-sepsis therapy. In some embodiments, methods provided in the present disclosure include administering a treatment to a subject based on measuring the fraction of CD45+ monocytes that are IL1R2^{hi}, HLA-DR^{lo}, and CD14+ in the subject.

In some embodiments, a method described in the present disclosure comprises administering a therapy, e.g., an antibiotic, intravenous fluids, vasopressors, surgery, oxygen, dialysis, and/or corticosteroids. In some embodiments, a method described in the present disclosure comprises administering an antibiotic. Examples of antibiotics include, but are not limited to, beta-lactams (e.g., penicillins, cephalosporins), aminoglycosides (e.g., streptomycin, neomycin, kanamycin, paromycin), chloramphenicol, glycopeptides (e.g., bleomycin, vancomycin, teicoplanin), ansamycins (e.g., geldanamycin, rifamycin, naphthomycin), streptogramins (e.g., pristinamycin), sulfonamides (e.g., prontosil, sulfanilamide, sulfadiazine, sulfisoxazole), tetracyclines (e.g., tetracycline, doxycycline, limecycline, oxytetracycline), macrolides (e.g., erythromycin, clarithromycin, azithromycin), oxazolidinones (e.g., linezolid, posizolid, tedizolid, cycloserine), quinolones (e.g., ciprofloxacin, leofloxain, trovafloxivin), and lipopeptides (e.g., daptomycin, surfactin).

In some embodiments, a method described in the present disclosure comprises administering a corticosteroid. Examples of corticosteroids include, but are not limited to, hydrocortisone, methylprednisolone, prednisolone, prednisone, triamcinolone, amcinonide, budesonide, desonide, fluocinolone acetonide, fluocinonide, halcinonide, triamcinolone acetonide, beclometasone, betamethasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate

An effective amount of an anti-sepsis therapy can be administered to a subject (e.g., a human) in need of the treatment via a suitable route, such as intravenous administration, e.g., as a bolus or by continuous infusion over a period of time, by intramuscular, intraperitoneal, intracerobrospinal, subcutaneous, intra-articular, intrasynovial, intrathecal, oral, inhalation, or topical routes.

“An effective amount” as used in the present disclosure refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. In some embodiments, it is preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for other reasons.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of sepsis. Alternatively, sustained continuous release formulations of therapeutic agent may be appropriate. Various formulations and devices for achieving sustained release are known in the art.

As used in the present disclosure, the term “treating” with respect to sepsis refers to the application or administration of a composition including one or more active agents to a subject, who has sepsis, or a symptom of sepsis, with the purpose to cure, heal, alleviate, relieve, alter, remedy, ameliorate, improve, or affect sepsis, or at least one symptom of sepsis.

Alleviating sepsis includes delaying the development or progression of sepsis, or reducing sepsis severity. Alleviating sepsis does not necessarily require curative results.

As used in the present disclosure, “delaying” the development of sepsis means to defer, hinder, slow, retard, stabilize, and/or postpone progression of sepsis. This delay can be of varying lengths of time, depending on the individuals being treated. A method that “delays” or alleviates the development of sepsis, or delays the onset of sepsis, is a method that reduces probability of developing one or more symptoms of sepsis in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of sepsis. Development of sepsis can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used in the present disclosure, “onset” or “occurrence” of sepsis includes initial onset and/or recurrence.

In some embodiments, the therapy is administered one or more times to the subject. The therapy, e.g., an antibiotic, intravenous fluids, vasopressors, surgery, oxygen, dialysis, and/or corticosteroids, may be administered along with another therapy as part of a combination therapy for treatment of sepsis.

The term combination therapy, as used in the present disclosure, embraces administration of these agents in a sequential manner, that is, in the present disclosure each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular, subcutaneous routes, and direct absorption through mucous membrane tissues. The agents can be administered by the same route or by different routes. For example, a first agent can be administered orally, and a second agent can be administered intravenously.

As used in the present disclosure, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used in the present disclosure, concurrent administration and substantially simultaneous administration are used interchangeably. Sequential administration refers to temporally separated administration of the agents described in the present disclosure.

Sequences:
Human resistin (RETN) transcript variant 1 DNA is provided by
NCBI Reference Sequence: NM_020415.4:
(SEQ ID NO: 1)
AAGAGGCCTC AAAGAAAGAG CTGCGGTGCA GGAATTCGTG TGCCGGATTT
GGTTAGCTGA GCCCACCGAG AGGCGCCTGC AGGATGAAAG CTCTCTGTCT
CCTCCTCCTC CCTGTCCTGG GGCTGTTGGT GTCTAGCAAG ACCCTGTGCT
CCATGGAAGA AGCCATCAAT GAGAGGATCC AGGAGGTCGC CGGCTCCCTA
ATATTTAGGG CAATAAGCAG CATTGGCCTG GAGTGCCAGA GCGTCACCTC
CAGGGGGGAC CTGGCTACTT GCCCCCGAGG CTTCGCCGTC ACCGGCTGCA
CTTGTGGCTC CGCCTGTGGC TCGTGGGATG TGCGCGCCGA GACCACATGT
CACTGCCAGT GCGCGGGCAT GGACTGGACC GGAGCGCGCT GCTGTCGTGT
GCAGCCCTGA GGTCGCGCGC AGCGCGTGCA CAGCGCGGGC GGAGGCGGCT
CCAGGTCCGG AGGGGTTGCG GGGGAGCTGG AAATAAACCT GGAGATGATG
ATGATGATGA TGATGA
Human resistin (RETN) transcript variant 2 DNA is provided by
NCBI Reference Sequence: NM_001193374.2:
(SEQ ID NO: 2)
AAGAGGCCTC AAAGAAAGAG CTGCGGTGCA GGAATTCGTG TGCCGGATTT
GGTTAGCTGA GCCCACCGAG AGGGATGAAA GCTCTCTGTC TCCTCCTCCT
CCCTGTCCTG GGGCTGTTGG TGTCTAGCAA GACCCTGTGC TCCATGGAAG
AAGCCATCAA TGAGAGGATC CAGGAGGTCG CCGGCTCCCT AATATTTAGG
GCAATAAGCA GCATTGGCCT GGAGTGCCAG AGCGTCACCT CCAGGGGGGA
CCTGGCTACT TGCCCCCGAG GCTTCGCCGT CACCGGCTGC ACTTGTGGCT
CCGCCTGTGG CTCGTGGGAT GTGCGCGCCG AGACCACATG TCACTGCCAG
TGCGCGGGCA TGGACTGGAC CGGAGCGCGC TGCTGTCGTG TGCAGCCCTG
AGGTCGCGCG CAGCGCGTGC ACAGCGCGGG CGGAGGCGGC TCCAGGTCCG
GAGGGGTTGC GGGGGAGCTG GAAATAAACC TGGAGATGAT GATGATGATG
ATGATGA
Human Interleukin-1 receptor type 2 (IL1R2) transcript variant 1
DNA is provided by NCBI Reference Sequence: NM_004633.4:
(SEQ ID NO: 3)
GCTGGAGGTG AAAGTCTGGC CTGGCAGCCT TCCCCAGGTG AGCAGCAACA
AGGCCACGTG CTGCTGGGTC TCAGTCCTCC ACTTCCCGTG TCCTCTGGAA
GTTGTCAGGA GCAATGTTGC GCTTGTACGT GTTGGTAATG GGAGTTTCTG
CCTTCACCCT TCAGCCTGCG GCACACACAG GGGCTGCCAG AAGCTGCCGG
TTTCGTGGGA GGCATTACAA GCGGGAGTTC AGGCTGGAAG GGGAGCCTGT
AGCCCTGAGG TGCCCCCAGG TGCCCTACTG GTTGTGGGCC TCTGTCAGCC
CCCGCATCAA CCTGACATGG CATAAAAATG ACTCTGCTAG GACGGTCCCA
GGAGAAGAAG AGACACGGAT GTGGGCCCAG GACGGTGCTC TGTGGCTTCT
GCCAGCCTTG CAGGAGGACT CTGGCACCTA CGTCTGCACT ACTAGAAATG
CTTCTTACTG TGACAAAATG TCCATTGAGC TCAGAGTTTT TGAGAATACA
GATGCTTTCC TGCCGTTCAT CTCATACCCG CAAATTTTAA CCTTGTCAAC
CTCTGGGGTA TTAGTATGCC CTGACCTGAG TGAATTCACC CGTGACAAAA
CTGACGTGAA GATTCAATGG TACAAGGATT CTCTTCTTTT GGATAAAGAC
AATGAGAAAT TTCTAAGTGT GAGGGGGACC ACTCACTTAC TCGTACACGA
TGTGGCCCTG GAAGATGCTG GCTATTACCG CTGTGTCCTG ACATTTGCCC
ATGAAGGCCA GCAATACAAC ATCACTAGGA GTATTGAGCT ACGCATCAAG
AAAAAAAAAG AAGAGACCAT TCCTGTGATC ATTTCCCCCC TCAAGACCAT
ATCAGCTTCT CTGGGGTCAA GACTGACAAT CCCGTGTAAG GTGTTTCTGG
GAACCGGCAC ACCCTTAACC ACCATGCTGT GGTGGACGGC CAATGACACC
CACATAGAGA GCGCCTACCC GGGAGGCCGC GTGACCGAGG GGCCACGCCA
GGAATATTCA GAAAATAATG AGAACTACAT TGAAGTGCCA TTGATTTTTG
ATCCTGTCAC AAGAGAGGAT TTGCACATGG ATTTTAAATG TGTTGTCCAT
AATACCCTGA GTTTTCAGAC ACTACGCACC ACAGTCAAGG AAGCCTCCTC
CACGTTCTCC TGGGGCATTG TGCTGGCCCC ACTTTCACTG GCCTTCTTGG
TTTTGGGGGG AATATGGATG CACAGACGGT GCAAACACAG AACTGGAAAA
GCAGATGGTC TGACTGTGCT ATGGCCTCAT CATCAAGACT TTCAATCCTA
TCCCAAGTGA AATAAATGGA ATGAAATAAT TCAAACACAA ACTCCGTACG
TCTTCTCTTA TGGAAGTGGC TGTGTCTTTT TGAGGGACTC TGTTCTTTGC
CTCAGTTGTC TACCAAAGGT GCCACATTTA TAGTGGCTTT GTAGTAAAGG
ACTAAAGTCT TA
Human Interleukin-1 receptor type 2 (IL1R2) transcript variant 2
DNA is provided by NCBI Reference Sequence: NR_048564.1:
(SEQ ID NO: 4)
GCAGAGTGGC ACAGTCACAT TCTAGAAGAC CATGTGGGAT GGGAGATACT
GTTGTGGTCA CCTCTGGAAA ATACATTCTG CTACTCTTAA AAACTAGTGA
CGCTCATACA AATCAACAGA AAGAGCTTCT GAAGGAAGAC TTTAAAGCTG
CTTCTGCCAC GTGCTGCTGG GTCTCAGTCC TCCACTTCCC GTGTCCTCTG
GAAGTTGTCA GGAGCAATGT TGCGCTTGTA CGTGTTGGTA ATGGGAGTTT
CTGCCTTCAC CCTTCAGCCT GCGGCACACA CAGGGGCTGC CAGAAGCTGC
CGGTTTCGTG GGAGGCATTA CAAGCGGGAG TTCAGGCTGG AAGGGGAGCC
TGTAGCCCTG AGGTGCCCCC AGGTGCCCTA CTGGTTGTGG GCCTCTGTCA
GCCCCCGCAT CAACCTGACA TGGCATAAAA ATGACTCTGC TAGGACGGTC
CCAGGAGAAG AAGAGACACG GATGTGGGCC CAGGACGGTG CTCTGTGGCT
TCTGCCAGCC TTGCAGGAGG ACTCTGGCAC CTACGTCTGC ACTACTAGAA
ATGCTTCTTA CTGTGACAAA ATGTCCATTG AGCTCAGAGT TTTTGAGAAT
ACAGATGCTT TCCTGCCGTT CATCTCATAC CCGCAAATTT TAACCTTGTC
AACCTCTGGG GTATTAGTAT GCCCTGACCT GAGTGAATTC ACCCGTGACA
AAACTGACGT GAAGATTCAA TGGTACAAGG ATTCTCTTCT TTTGGATAAA
GACAATGAGA AATTTCTAAG TGTGAGGGGG ACCACTCACT TACTCGTACA
CGATGTGGCC CTGGAAGATG CTGGCTATTA CCGCTGTGTC CTGACATTTG
CCCATGAAGG CCAGCAATAC AACATCACTA GGAGTATTGA GCTACGCATC
AAGAAAAAAA AAGAAGAGAC CATTCCTGTG ATCATTTCCC CCCTCAAGAC
CATATCAGCT TCTCTGGGGT CAAGACTGAC AATCCCGTGT AAGGTGTTTC
TGGGAACCGG CACACCCTTA ACCACCATGC TGTGGTGGAC GGCCAATGAC
ACCCACATAG AGAGCGCCTA CCCGGGAGGC CGCGTGACCG AGGGGCCACG
CCAGGAATAT TCAGAAAATA ATGAGAACTA CATTGAAGTG CCATTGATTT
TTGATCCTGT CACAAGAGAG GATTTGCACA TGGATTTTAA ATGTGTTGTC
CATAATACCC TGAGTTTTCA GACACTACGC ACCACAGTCA AGGAAGCCTC
CTCCACGTTC TCCTGGGGCA TTGTGCTGGC CCCACTTTCA CTGGCCTTCT
TGGTTTTGGG GGGAATATGG ATGCACAGAC GGTGCAAACA CAGAACTGGA
AAAGCAGATG GTCTGACTGT GCTATGGCCT CATCATCAAG ACTTTCAATC
CTATCCCAAG TGAAATAAAT GGAATGAAAT AATTCAAACA CAAAAAAAAA
AAAAAAAAAA AAA
Human Interleukin-1 receptor type 2 (IL1R2) transcript variant 3
DNA is provided by NCBI Reference Sequence: NM_001261419.2:
(SEQ ID NO: 5)
GCTGGAGGTG AAAGTCTGGC CTGGCAGCCT TCCCCAGGTG AGCAGCAACA
AGGCCACGTG CTGCTGGGTC TCAGTCCTCC ACTTCCCGTG TCCTCTGGAA
GTTGTCAGGA GCAATGTTGC GCTTGTACGT GTTGGTAATG GGAGTTTCTG
CCTTCACCCT TCAGCCTGCG GCACACACAG GGGCTGCCAG AAGCTGCCGG
TTTCGTGGGA GGCATTACAA GCGGGAGTTC AGGCTGGAAG GGGAGCCTGT
AGCCCTGAGG TGCCCCCAGG TGCCCTACTG GTTGTGGGCC TCTGTCAGCC
CCCGCATCAA CCTGACATGG CATAAAAATG ACTCTGCTAG GACGGTCCCA
GGAGAAGAAG AGACACGGAT GTGGGCCCAG GACGGTGCTC TGTGGCTTCT
GCCAGCCTTG CAGGAGGACT CTGGCACCTA CGTCTGCACT ACTAGAAATG
CTTCTTACTG TGACAAAATG TCCATTGAGC TCAGAGTTTT TGAGAATACA
GATGCTTTCC TGCCGTTCAT CTCATACCCG CAAATTTTAA CCTTGTCAAC
CTCTGGGGTA TTAGTATGCC CTGACCTGAG TGAATTCACC CGTGACAAAA
CTGACGTGAA GATTCAATGG TACAAGGATT CTCTTCTTTT GGATAAAGAC
AATGAGAAAT TTCTAAGTGT GAGGGGGACC ACTCACTTAC TCGTACACGA
TGTGGCCCTG GAAGATGCTG GCTATTACCG CTGTGTCCTG ACATTTGCCC
ATGAAGGCCA GCAATACAAC ATCACTAGGA GTATTGAGCT ACGCATCAAG
AAAAAAAAAG AAGAGACCAT TCCTGTGATC ATTTCCCCCC TCAAGACCAT
ATCAGCTTCT CTGGGGTCAA GACTGACAAT CCCGTGTAAG GTGTTTCTGG
GAACCGGCAC ACCCTTAACC ACCATGCTGT GGTGGACGGC CAATGACACC
CACATAGAGA GCGCCTACCC GGGAGGCCGC GTGACCGAGG GGCCACGCCA
GTAAGTGGGC CAGGGTCTTC TGTTGAGAAC TCTGTGGGTT TCGCTCTTCC
TTTTGGAGAC AGTTATCACT ATGACCCACA TACCACATTA AAAGTTACTT
TTTTTGATTC CAAACTGTTG GATGTTTAGA ATTTAAAAAA TTGTATTTTG
CTAAAAAT
Human clusterin (CLU) transcript variant 1 DNA is provided by
NCBI Reference Sequence: NM_001831.4:
(SEQ ID NO: 6)
GCGGCGTCGC CAGGAGGAGC GCGCGGGCAC AGGGTGCCGC TGACCGAGGC
GTGCAAAGAC TCCAGAATTG GAGGCATGAT GAAGACTCTG CTGCTGTTTG
TGGGGCTGCT GCTGACCTGG GAGAGTGGGC AGGTCCTGGG GGACCAGACG
GTCTCAGACA ATGAGCTCCA GGAAATGTCC AATCAGGGAA GTAAGTACGT
CAATAAGGAA ATTCAAAATG CTGTCAACGG GGTGAAACAG ATAAAGACTC
TCATAGAAAA AACAAACGAA GAGCGCAAGA CACTGCTCAG CAACCTAGAA
GAAGCCAAGA AGAAGAAAGA GGATGCCCTA AATGAGACCA GGGAATCAGA
GACAAAGCTG AAGGAGCTCC CAGGAGTGTG CAATGAGACC ATGATGGCCC
TCTGGGAAGA GTGTAAGCCC TGCCTGAAAC AGACCTGCAT GAAGTTCTAC
GCACGCGTCT GCAGAAGTGG CTCAGGCCTG GTTGGCCGCC AGCTTGAGGA
GTTCCTGAAC CAGAGCTCGC CCTTCTACTT CTGGATGAAT GGTGACCGCA
TCGACTCCCT GCTGGAGAAC GACCGGCAGC AGACGCACAT GCTGGATGTC
ATGCAGGACC ACTTCAGCCG CGCGTCCAGC ATCATAGACG AGCTCTTCCA
GGACAGGTTC TTCACCCGGG AGCCCCAGGA TACCTACCAC TACCTGCCCT
TCAGCCTGCC CCACCGGAGG CCTCACTTCT TCTTTCCCAA GTCCCGCATC
GTCCGCAGCT TGATGCCCTT CTCTCCGTAC GAGCCCCTGA ACTTCCACGC
CATGTTCCAG CCCTTCCTTG AGATGATACA CGAGGCTCAG CAGGCCATGG
ACATCCACTT CCATAGCCCG GCCTTCCAGC ACCCGCCAAC AGAATTCATA
CGAGAAGGCG ACGATGACCG GACTGTGTGC CGGGAGATCC GCCACAACTC
CACGGGCTGC CTGCGGATGA AGGACCAGTG TGACAAGTGC CGGGAGATCT
TGTCTGTGGA CTGTTCCACC AACAACCCCT CCCAGGCTAA GCTGCGGCGG
GAGCTCGACG AATCCCTCCA GGTCGCTGAG AGGTTGACCA GGAAATACAA
CGAGCTGCTA AAGTCCTACC AGTGGAAGAT GCTCAACACC TCCTCCTTGC
TGGAGCAGCT GAACGAGCAG TTTAACTGGG TGTCCCGGCT GGCAAACCTC
ACGCAAGGCG AAGACCAGTA CTATCTGCGG GTCACCACGG TGGCTTCCCA
CACTTCTGAC TCGGACGTTC CTTCCGGTGT CACTGAGGTG GTCGTGAAGC
TCTTTGACTC TGATCCCATC ACTGTGACGG TCCCTGTAGA AGTCTCCAGG
AAGAACCCTA AATTTATGGA GACCGTGGCG GAGAAAGCGC TGCAGGAATA
CCGCAAAAAG CACCGGGAGG AGTGAGATGT GGATGTTGCT TTTGCACCTA
CGGGGGCATC TGAGTCCAGC TCCCCCCAAG ATGAGCTGCA GCCCCCCAGA
GAGAGCTCTG CACGTCACCA AGTAACCAGG CCCCAGCCTC CAGGCCCCCA
ACTCCGCCCA GCCTCTCCCC GCTCTGGATC CTGCACTCTA ACACTCGACT
CTGCTGCTCA TGGGAAGAAC AGAATTGCTC CTGCATGCAA CTAATTCAAT
AAAACTGTCT TGTGAGCTGA TCGCTTGGAG GGTCCTCTTT TTATGTTGAG
TTGCTGCTTC CCGGCATGCC TTCATTTTGC TATGGGGGGC AGGCAGGGGG
GATGGAAAAT AAGTAGAAAC AAAAAAGCAG TGGCTAAGAT GGTATAGGGA
CTGTCATACC AGTGAAGAAT AAAAGGGTGA AGAATAAAAG GGATATGATG
ACAAGGTTGA TCCACTTCAA GAATTGCTTG CTTTCAGGAA GAGAGATGTG
TTTCAACAAG CCAACTAAAA TATATTGCTG CAAATGGAAG CTTTTCTGTT
CTATTATAAA ACTGTCGATG TATTCTGACC AAGGTGCGAC AATCTCCTAA
AGGAATACAC TGAAAGTTAA GGAGAAGAAT CAGTAAGTGT AAGGTGTACT
TGGTATTATA ATGCATAATT GATGTTTTCG TTATGAAAAC ATTTGGTGCC
CAGAAGTCCA AATTATCAGT TTTATTTGTA AGAGCTATTG CTTTTGCAGC
GGTTTTATTT GTAAAAGCTG TTGATTTCGA GTTGTAAGAG CTCAGCATCC
CAGGGGCATC TTCTTGACTG TGGCATTTCC TGTCCACCGC CGGTTTATAT
GATCTTCATA CCTTTCCCTG GACCACAGGC GTTTCTCGGC TTTTAGTCTG
AACCATAGCT GGGCTGCAGT ACCCTACGCT GCCAGCAGGT GGCCATGACT
ACCCGTGGTA CCAATCTCAG TCTTAAAGCT CAGGCTTTTC GTTCATTAAC
ATTCTCTGAT AGAATTCTGG TCATCAGATG TACTGCAATG GAACAAAACT
CATCTGGCTG CATCCCAGGT GTGTAGCAAA GTCCACATGT AAATTTATAG
CTTAGAATAT TCTTAAGTCA CTGTCCCTTG TCTCTCTTTG AAGTTATAAA
CAACAAACTT AAAGCTTAGC TTATGTCCAA GGTAAGTATT TTAGCATGGC
TGTCAAGGAA ATTCAGAGTA AAGTCAGTGT GATTCACTTA ATGATATACA
TTAATTAGAA TTATGGGGTC AGAGGTATTT GCTTAAGTGA TCATAATTGT
AAAGTATATG TCACATTGTC ACATTAATGT CACACTGTTT CAAAAGTTA

Human Interleukin-1 receptor type 2 (IL1R2) transcript variant 2 DNA is provided by NCBI Reference Sequence: NR_048564.1:

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited in the present disclosure are incorporated by reference for the purposes or subject matter referenced in the present disclosure.

### EXAMPLES

In order that the invention described in the present disclosure may be more fully understood, the following examples are set forth. The examples described in this application are offered to illustrate the systems and methods provided in the present disclosure and are not to be construed in any way as limiting their scope.

### Example 1: Methods and Experimental Design

### Study Samples and Clinical Adjudication

Primary cohorts comprised subjects with UTI and urosepsis who presented to the ED at the Massachusetts General Hospital (MGH), and secondary cohorts were hospitalized subjects with and without sepsis on inpatient services at the Brigham and Women's Hospital (BWH); both hospitals are located in Boston, Mass. Informed consent was obtained from subjects or their surrogates. Blood samples from these subjects and healthy controls were drawn with EDTA Vacutainer tubes (BD Biosciences) and processed within 3 hours of collection. De-identified BMMC samples were purchased from AllCells or Stemcell Technologies.

The primary cohorts were enrolled in the ED at the Massachusetts General Hospital (MGH) from December 2017 to November 2018. They consisted of people with UTI, defined by a urine white blood cell count of >20 per high-power field on clinical urinalysis. Study samples were collected within 12 hours of subject arrival to the ED. Individuals with UTI were initially enrolled into one of two categories: (1) those with leukocytosis (blood WBC≥12,000 per mm³) without another cause, indicating systemic inflammation from the UTI, but without organ dysfunction (cohort Leuk-UTI), and (2) those with organ dysfunction, which defines urosepsis. For the urosepsis group, subjects were recruited who met UTI criteria in the presence of organ dysfunction, as specified in national quality measure definitions that are adapted from Sepsis-2 consensus definitions, specifically systolic blood pressure <90 mmHg, lactate >2.0 mg dl⁻¹, requirement for vasopressor medication, new Glasgow coma score (GCS)<15 denoting altered mental status, new creatinine >2.0 mg dl⁻¹ or need for mechanical ventilation. SOFA scores were calculated, but they were not a specific criterion for enrollment or adjudication.

Once the results of initial diagnostics sent in the course of routine clinical care, including cultures, were available and the subsequent clinical course during hospitalization was known (that is, at least 48 hours after initial presentation), clinical adjudication of each enrolled subject was independently performed by three investigators, blinded to research analysis outcomes. Each enrolled subject who was found to meet criteria for the study was adjudicated to one of three clinical categories: Leuk-UTI, Int-URO and URO. Given the spectrum of organ dysfunction severity among enrolled patients, mild or transient organ dysfunction (intermediate urosepsis, or Int-URO) and sustained infection-related organ dysfunction (urosepsis, or URO) were differentiated. Int-URO included subjects with physiologic perturbations that qualify as sepsis in the setting of infection per national quality measure and Sepsis-2 consensus definitions, but for whom observed organ dysfunction was isolated and relatively mild, and resolved quickly with initial therapies. Examples included hypotension that resolved with fluid resuscitation, isolated mild elevation in creatinine that normalized within 24 hours or elevated initial lactate or alteration in mental status that improved within 4-6 hours. URO included subjects with organ dysfunction that persisted or worsened despite initial therapy. Examples included refractory hypotension requiring vasopressor support, persistent renal dysfunction >24 hours after enrollment, lactate increasing despite adequate volume resuscitation or multiple organ-system dysfunction. Discrepancies in adjudication among the three clinicians were resolved as a group.

Enrollment of patients: For the category Leuk-UTI, enrollment of subjects with UTI with systemic response but without sepsis was specifically targeted so as to provide the most appropriate comparison for urosepsis cohorts, as a comparison with subjects with simple UTI without evidence of a systemic response might highlight host signature differences attributable to a systemic response to localized infection rather than being specific to sepsis. To obtain as pure an immune signature for infection as possible, individuals with immunodeficiencies were excluded, including HIV, concurrent immunomodulatory drug therapy (including prednisone or steroid equivalent, chemotherapy, or biologic immunomodulators), recipients of bone-marrow or solid-organ transplantation and individuals with autoimmune disease. Of note, two subjects in the Leuk-UTI cohort were asplenic. For all these primary cohorts (Leuk-UTI, Int-URO and URO), patients who had received their first intravenous antibiotic >12 hours prior to enrollment were excluded. Of the 27 people enrolled in these cohorts, 7 were enrolled prior to antibiotic initiation, and 20 were enrolled within 7 hours of antibiotic initiation, with the median time to enrollment from antibiotic initiation for all enrolled patients 1.1 hours (IQR, 0.2-2.4 hours).

Uninfected control samples for the primary cohorts were obtained from two sources. First, follow-up blood samples were obtained from four primary cohort patients at 2-3 months after index enrollment (2 Leuk-UTI and 2 URO subjects). For all other primary cohort subjects, uninfected control samples consisted of blood samples from age-, gender- and ethnicity-matched healthy controls obtained from Research Blood Components (Watertown, Mass.).

Secondary cohorts consisted of hospitalized subjects identified as having bacteremia and sepsis but not requiring ICU admission (Bac-SEP), subjects with sepsis requiring ICU care (ICU-SEP) and subjects in the ICU for conditions other than sepsis (ICU-NoSEP). These cohorts were enrolled in the Brigham and Women's Hospital (BWH) as part of the Registry of Critical Illness. The criteria for subject recruitment for this cohort were described in Nakahira et al., PLos Med. 10, e1001577 (2013). discussion e1001577 and Dolinay et al., Am. J. Respir. Crit. Care Med. 185, 1225-1234 (2012), which contents are incorporated by reference in the present disclosure.

The Bac-SEP subjects were recruited between December 2017 and September 2018 from hospital inpatient floors (not ICU) and had positive blood cultures within 24 hours of sample collection (excluding those blood cultures that grew coagulase-negative Staphylococcus species, which was considered likely to be a contaminant). The ICU-SEP and ICU-NoSEP subjects were enrolled in the BWH ICU between November 2017 and October 2018.

In contrast to the primary cohorts enrolled in the MGH ED, most subjects in the secondary cohorts were enrolled 2-3 days after initial presentation of disease and initiation of therapy, with all subjects enrolled >24 hours from hospital presentation. Most subjects had therefore received antibiotics for >24 hours prior to enrollment (median, 70 hours for Bac-SEP, IQR: 61-79 hours; median, 49 hours for ICU-SEP, IQR: 44-65 h). The sources of infection for secondary-cohort subjects included pulmonary, urinary, intraabdominal and endovascular sites. To ensure consistency of adjudication among cohorts, secondary cohorts were adjudicated for the presence of sepsis by the three adjudicators who adjudicated the primary cohort, employing the same methods used for the primary cohorts.

During the index illnesses and/or hospitalizations, there were no deaths among subjects in the Leuk-UTI, Int-URO, BAC-SEP and ICU-NoSEP cohorts, and there was one death in each group among subjects in the URO and ICU-SEP cohorts. Given the small numbers of deaths, the potential significance of these death incidences was not specifically analyzed.

Isolation and Cryopreservation of PBMCs from Whole Blood

Cells were isolated from whole-blood samples using density-gradient centrifugation, as described in a previous study (Reyes et al., Sci. Adv. 5, eaau9223 (2019)). Briefly, whole blood was diluted 1:1 with 1×PBS, layered on top of Ficoll-Paque Plus (GE Healthcare), and centrifuged at 1,200 g for 20 min. The PBMC layer was resuspended in 10 ml RPMI-1640 (Gibco), and centrifuged again at 300 g for 10 min. The cells were counted, resuspended in Cryostor CS10 (StemCell Technologies) and aliquoted in 1.5 ml cryopreservation tubes at a concentration of 2×10⁶ cells per milliliter. The tubes were kept at −80° C. overnight, then transferred to liquid nitrogen for long-term storage. The plasma layer from density gradient separation was also collected, aliquoted in 1-ml tubes and stored at −80° C.

### Staining, Flow Cytometry and Dendritic-Cell Enrichment

Samples were processed in batches of six or eight for pooling in single-cell RNA sequencing runs. All cells were stained with a general panel: DAPI, CD3-APC (HIT3a), CD19-APC (HIB19), CD20-APC (2H7), CD56-APC (5.1H11), CD14-FITC (M5E2), CD16-AF700 (B73.1), CD45-PE-Cy7 (HI30) and HLA-DR-PE (L243) (BioLegend). At the same time, 10 of cell-hashing antibody (HTO) was added to each sample (BioLegend). Samples were run on a SH800 cell sorter (Sony) to obtain flow-cytometry data and sort both live CD45+ cells and dendritic cells. For samples from subjects enrolled in the MGH ED, dendritic cells were enriched separately with a MACS human pan-DC enrichment kit (Miltenyi Biotec). For sorting MS1 cells, the following panel was used: DAPI, CD3-APC (HIT3a), CD19-APC (HIB19), CD20-APC (2H7), CD56-APC (5.1H11), CD14-FITC (M5E2), CD45-AF700 (HI30), HLA-DR-PE-Cy7 (L243) (BioLegend) and IL1R2-PE (34141, ThermoFisher Scientific).

### Single-Cell RNA-Seq and Analysis

Single-cell RNA-seq was performed on the Chromium platform, using the single cell expression 3′ v2 profiling chemistry (10× Genomics) combined with cell hashing. HTO-labeled cells from 6-8 donors were pooled equally then washed twice with RPMI-1640 immediately before loading on the 10× controller. Complementary DNA amplification and library construction were conducted following the manufacturer's protocol, with additional steps for the amplification of HTO barcodes. Libraries were sequenced to a depth of ˜50,000 reads per cell on a Novaseq S2 (I lumina). The data were aligned to the GRCh38 reference genome using cellranger v2.1 (10× Genomics), and the hashed cells were demultiplexed using the CITE-seq count tool (https://github.com/Hoohm/CITE-seq-Count).

Single-cell data analysis was performed using scanpy. Count matrices from the cellranger output were preprocessed by filtering for cells and genes (minimum cells per gene, 10; minimum UMI per cell, 100). Before clustering, the full dataset or a subset thereof was filtered for highly variable genes (minimum mean, 0.0125 and dispersion, 0.5 per gene) and scaled. Clustering was performed on the top 50 principal components of the data using the Leiden algorithm with varying resolution. To quantify the robustness of each clustering solution, the data were subsampled without replacement (90% of cells, 20 iterations) and re-clustered, and an adjusted Rand index was then computed between the solutions for the original and subsampled data. The highest resolution at which the robustness began to decrease was chosen for further analysis. To ensure that no subject- or batch-specific clusters were included in the data, small clusters (<500 cells) were combined with the next closest cluster on the basis of their similarity in gene-expression profiles. Differentially expressed genes were determined for each state by a Wilcoxon rank-sum test, with an FDR cutoff of 0.01. For visualization, t-SNE projections were computed on the top 10 principal components of the dataset or subsets thereof. To specifically find genes that distinguish between ICU-SEP and ICU-NoSEP populations, differentially expressed genes were filtered for those that have an in-group fraction >0.4 and out-group fraction <0.6. Consensus non-negative matrix factorization analysis was performed as detailed in Kotliar et al., Elife 8, 310599 (2019). To ensure that no subject- or batch-specific modules were analyzed, only gene programs with a mean usage >50 across all subjects were included for further analysis.

Subject Classification and Comparison with Published Predictors

All comparison of abundances was tested for significance by a Wilcoxon rank-sum test. Benjamini-Hochberg FDR correction was applied to the calculated P values for multiple testing of either cell types or states. To compare against published gene-based predictors, UMI counts were summed for each gene from all cells for each subject, scaled to the total number of UMI counts per patient, and calculated the FAIM-to-PLAC8 ratio, SeptiCyte Lab and Sepsis Metascore following published protocols. ROC curves were plotted on the basis of these absolute scores, as well as the fraction of MS1 for each subject.

### Bulk-Data Deconvolution, Gene-Signature Mapping and Meta-Analysis

A reference signature matrix for cell states was identified by generating bulk profiles from single-cell references, and ranking the genes based on effect size. The number of genes was optimized in the signature matrix by finding the minimum number of genes where the reduction in prediction error is saturated. The value was to be at >50 genes and selected 100 genes per state and lineage (1,201 total, union of all genes) in the final matrix. To construct the signature matrix, UMI counts for each state was summed, normalized to the number of total UMIs per state and quantile-normalized the resulting matrix.

Datasets comparing sepsis and healthy controls were obtained as outlined in two published studies (Sweeny et al., Crit. Care Med. 46, 915-925 (2018) and Sweeny et al., Crit. Care Med. 45, 1-10 (2017)). Datasets with gene-expression matrices that were not publicly available were not included in the analysis. Gene-expression deconvolution was performed using CIBERSORT. Noting that the state signatures only captured PBMC states and excluded high-density cells in whole blood, the data were deconvolved with a no-sum-to-one constraint and absolute scoring. The resulting score matrix was then used as an input to MetaIntegrator. The effect size of each state was visualized using forest plots, and the classification performance of MS1 cells was quantified by generating a summary ROC plot.

### Stimulation of Bone Marrow and Peripheral Blood Cells

For MS1-induction experiments, bone marrow or peripheral mononuclear cells were cultured in SFEM II supplemented with 1×CC110 (StemCell Technologies) with or without the presence of 100 ng ml⁻¹ LPS or Pam3CSK4 (Invivogen) for up to 4 days. For restimulation experiments, sorted monocytes were rested for 24 hours in RPMI-1640 supplemented with 10% heat-inactivated FBS and 1× penicillin-streptomycin (Gibco), before adding 100 ng ml⁻¹ LPS (Invivogen).

### ATAC-Seq Processing and Data Analysis

ATAC-seq was performed on 25,000 sorted cells, as described in a published protocol (Corces et al., Nat. Methods 14, 959-962 (2017)). Libraries were sequenced on a NextSeq (I lumina) with 38×38 paired-end reads and at least 10 million reads per sample. Sequencing data were aligned using the ENCODE Project ATAC-seq pipeline (https://www.encodeproject.org/atac-seq/), and further analyzed using custom scripts. To generate a peak count matrix, a consensus peak set using the ‘multiinter’ function was first identified, and then analyzed the number of counts for each sample using the function ‘coverageBed’ from bedtools v2. Differential peak analysis was performed using edgeR, using the peak count matrix as input. Peak motifs were analyzed using the ‘findMotifsGenome’ function in Homer v4.1, with a window size of 200 bp.

### Bulk RNA-Seq Processing and Data Analysis

Bulk RNA-seq was performed using Smart-Seq2 (Picelli et al., Nat. Protoc. 9, 171-181 (2014)) with minor modifications, as described in a previous study (Reyes et al., Sci. Adv. 5, eaau9223 (2019)). Briefly, 5,000 sorted or cultured cells were resuspended in 15 μl of Buffer TCL (Qiagen), and their RNA was purified by a 2.2×SPRI cleanup with RNAClean XP magnetic beads (Agencourt). After reverse transcription, amplification and cleanup, libraries were quantified using a Qubit fluorometer (Invitrogen), and their size distributions were determined using an Agilent Bioanalyzer 2100. Amplicon concentrations were normalized to 0.1 ng ml⁻¹ and sequencing libraries were constructed using a Nextera XT DNA Library Prep Kit (Illumina), following the manufacturer's protocol. All RNA-seq libraries were sequenced with 38×38 paired-end reads using a NextSeq (Illumina). RNA-seq libraries were sequenced to a depth of >2 million reads per sample. STAR was used to align sequencing reads to the UCSC hg19 transcriptome and RSEM was used to generate an expression matrix for all samples. Both raw count and transcripts per million data were analyzed using edgeR and custom python scripts. The list of identified receptor-ligand pairs was obtained from a previous publication (Ramilowski et al., Nat. Commun. 6, 7866 (2015)).

### Cytokine Measurements

Culture supernatants were diluted 2× in PBS and frozen at −80° C. before processing. Samples from multiple experiments were thawed and analyzed in parallel using the Legendplex Human Inflammation Panel, TNF-α (BioLegend). Flow cytometry data were acquired on a Cytoflex LX (Beckman Coulter) and analyzed using FlowJo v10.1.

### Example 2. scRNA-Seq Defines Immune Cell States in Sepsis Patients Across Multiple Clinical Cohorts

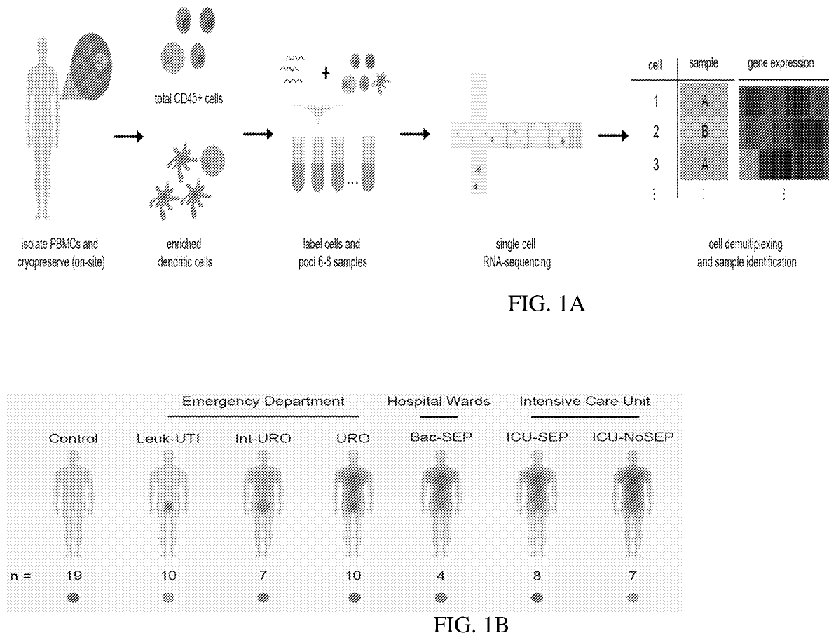

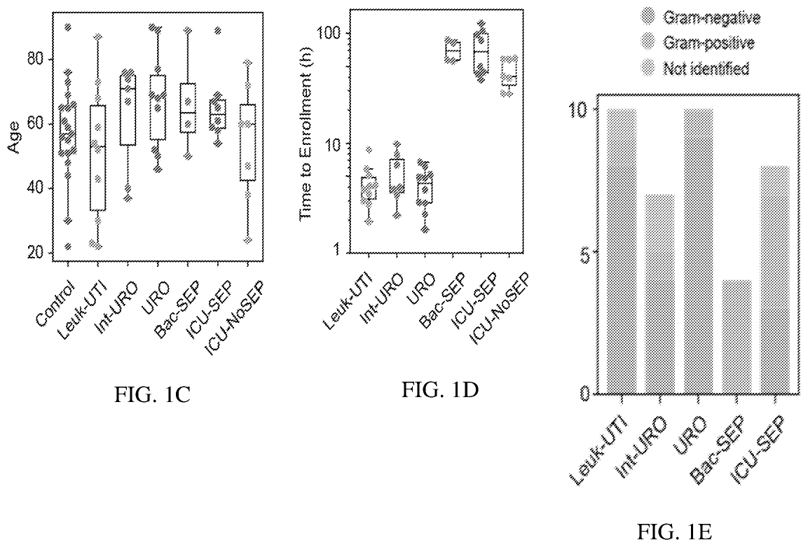

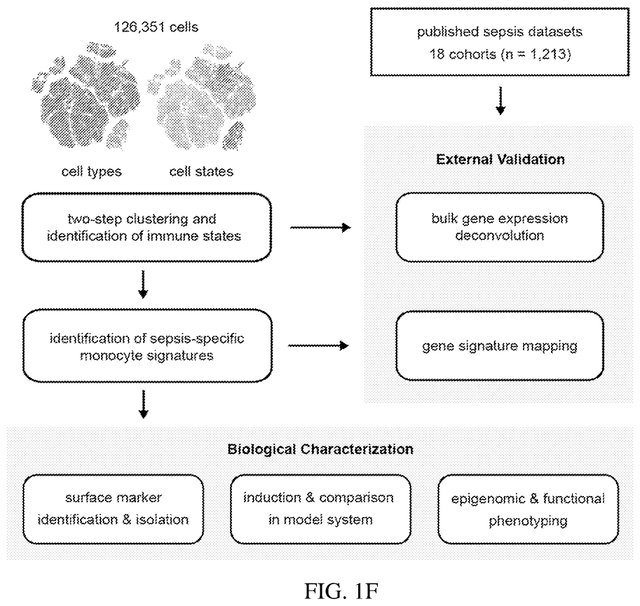

Single-cell RNA sequencing (scRNA-seq) was performed on PBMCs from people with sepsis and controls to define the range of cell states present in these subjects, to identify differences in cell-state composition between groups and to detect immune signatures that distinguish sepsis from the normal immune response to bacterial infection (FIG. 1). The primary cohorts targeted subjects with urinary-tract infection (UTI) early in their disease course, within 12 hours of presentation to the emergency department (ED) (FIG. 1B-1E and Table 1). UTI was selected to minimize heterogeneity introduced by different infectious sites and to maximize diagnostic clarity because a UTI can be reliably confirmed post hoc using a urine culture. Subjects with UTI (clinical urinalysis with >20 white blood cells per high-power field) were included as the primary infection both with and without signs of sepsis, and subsequently adjudicated the enrolled subjects into UTI with leukocytosis (blood WBC≥12,000 per mm3) but no organ dysfunction (Leuk-UTI), UTI with mild or transient organ dysfunction (Int-URO) and UTI with clear or persistent organ dysfunction (Urosepsis, URO). Subjects with simple UTI without leukocytosis or signs of organ dysfunction were not enrolled. The schema as described in the present disclosure distinguished transient versus sustained sepsis-related organ dysfunction, although both met established criteria (Sepsis-2 criteria) for sepsis.

Subjects from two secondary cohorts from a different hospital were profiled: bacteremic individuals with sepsis in hospital wards (Bac-SEP) and those admitted to the medical intensive care unit (ICU) either with sepsis (ICU-SEP) or without sepsis (ICU-NoSEP). Inclusion criteria were the same for primary and secondary cohorts. These secondary cohorts included people later in their disease course, who enrolled at least 24 hours after initial hospital presentation and receipt of intravenous antibiotics. For comparison, specimens from uninfected, healthy controls (Control) were analyzed. The multi-cohort approach, spanning two hospitals and several clinical phenotypes, supported the generalizability of the results across different clinical contexts.

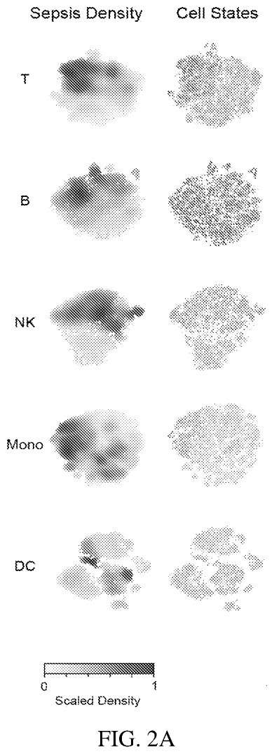

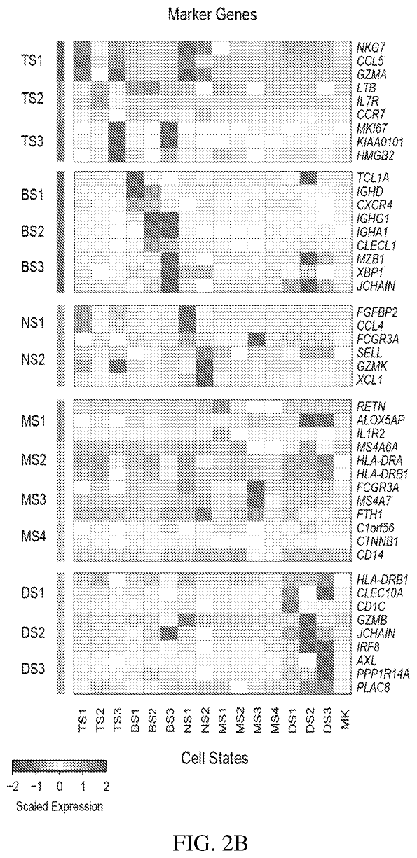

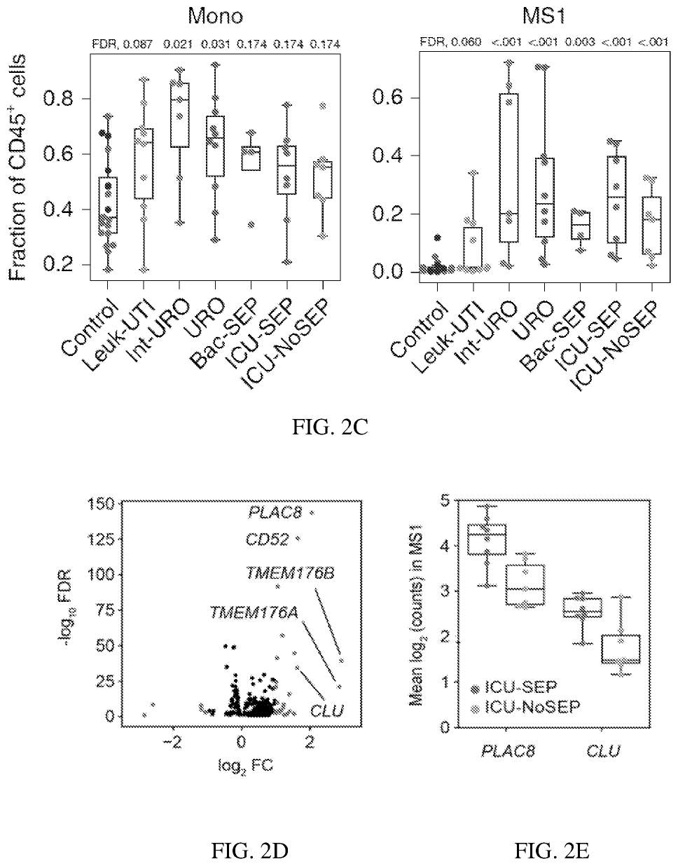

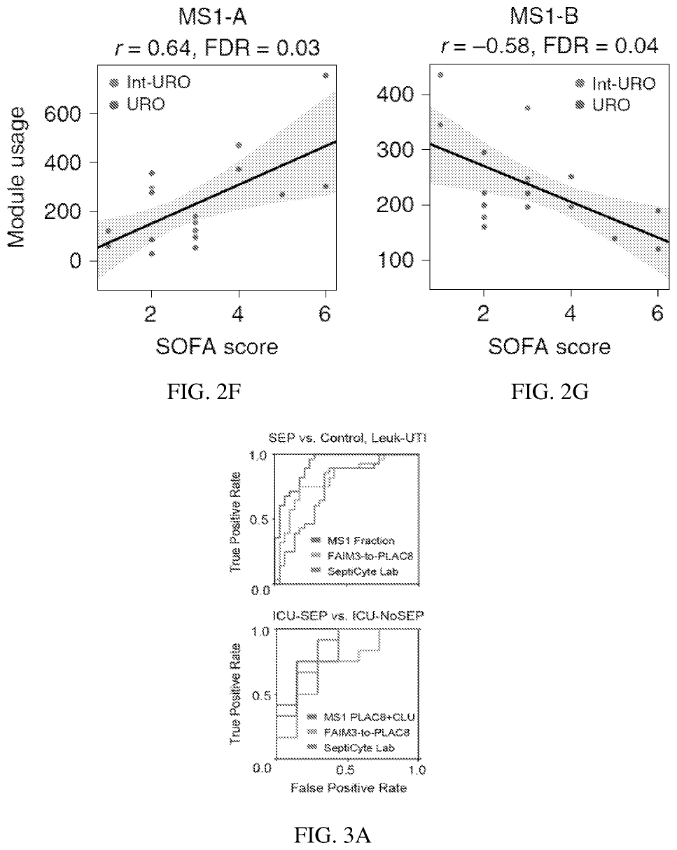

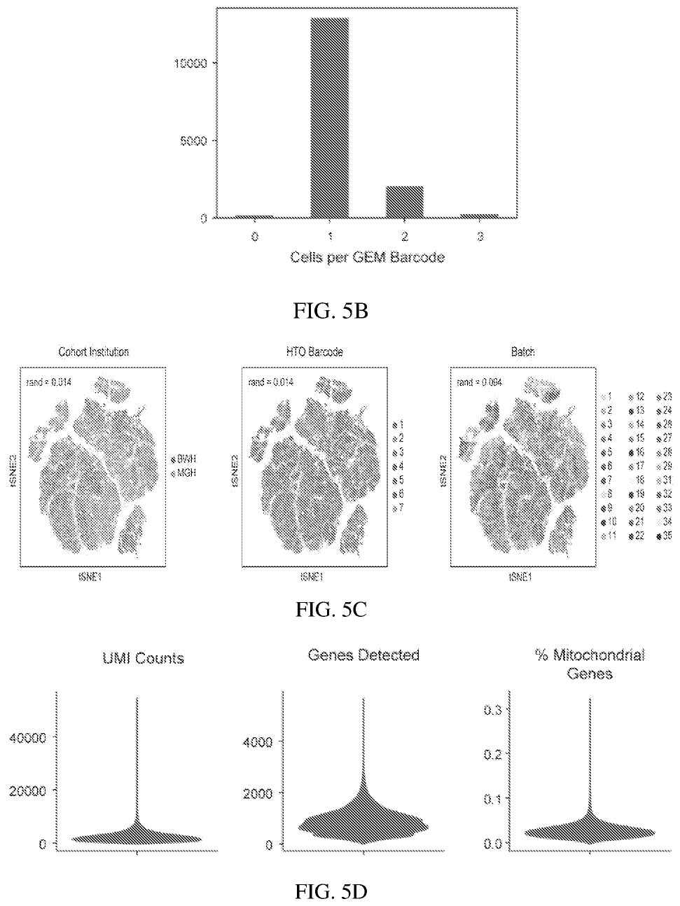

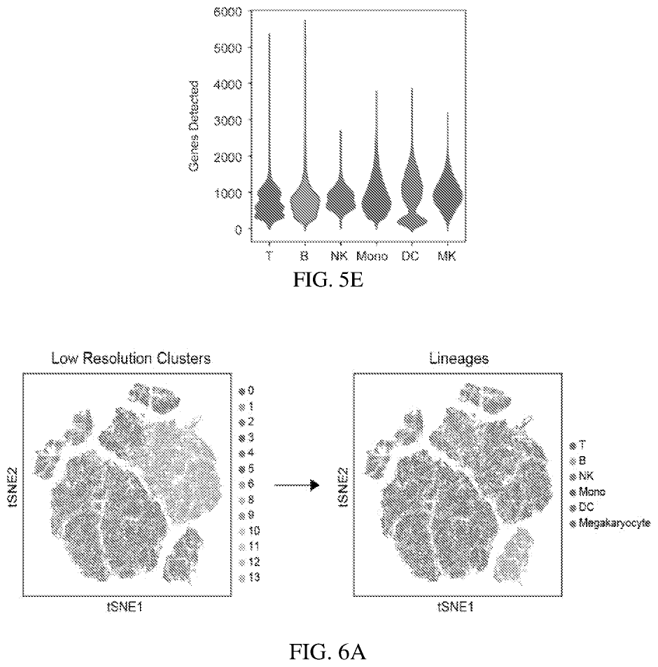

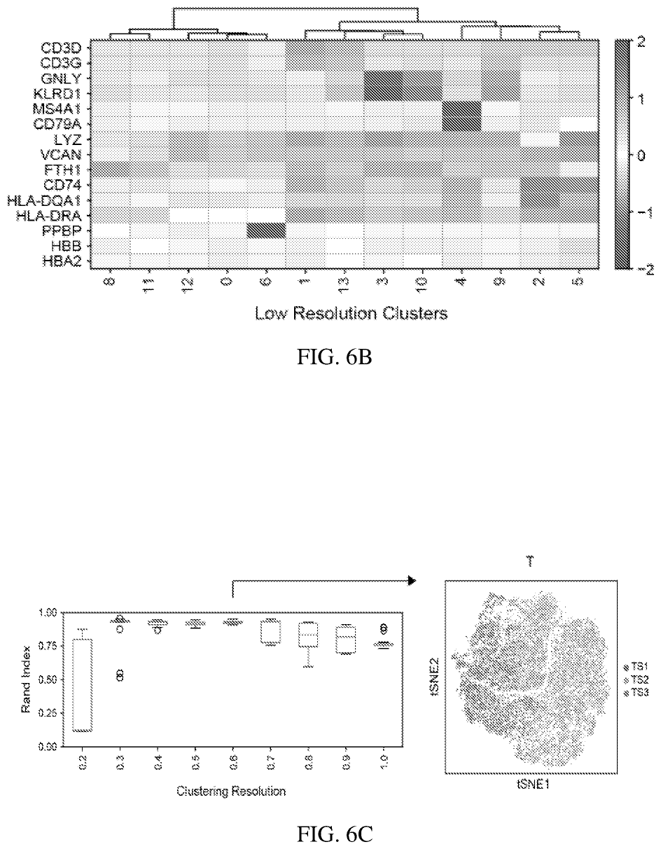

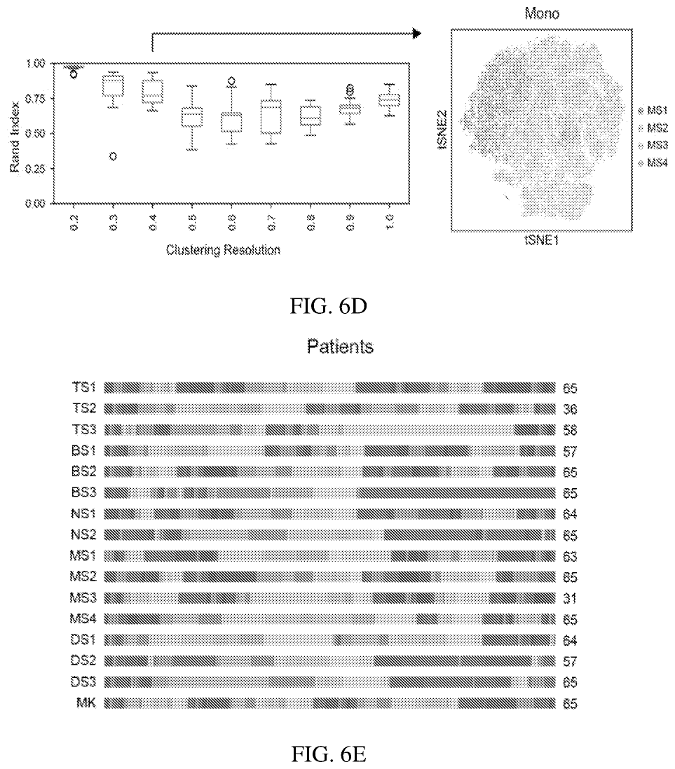

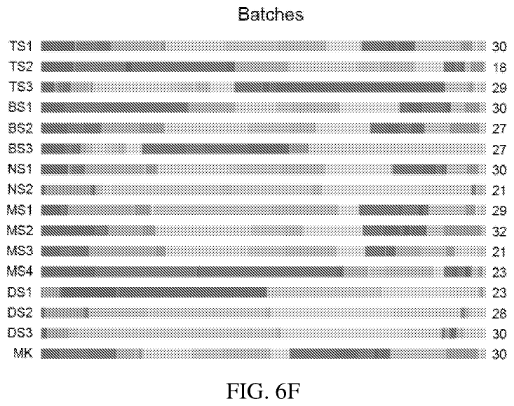

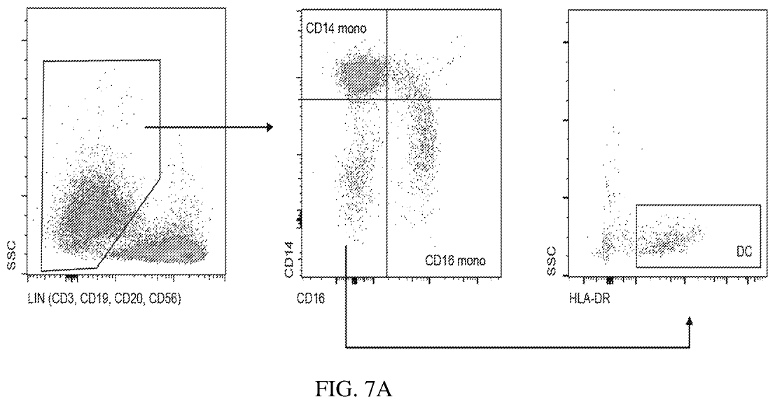

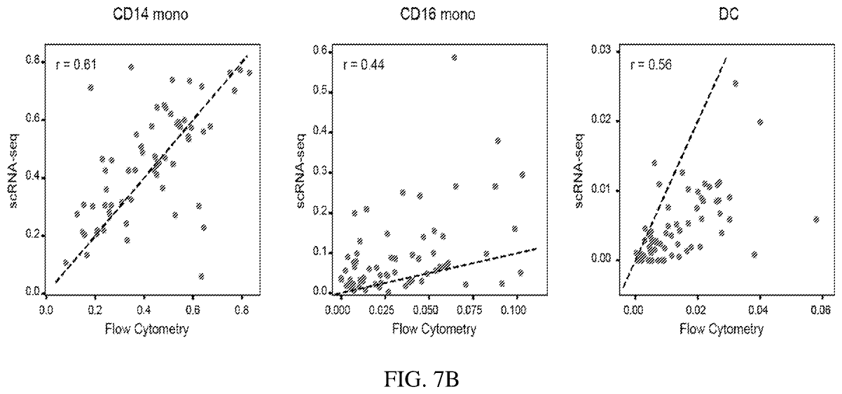

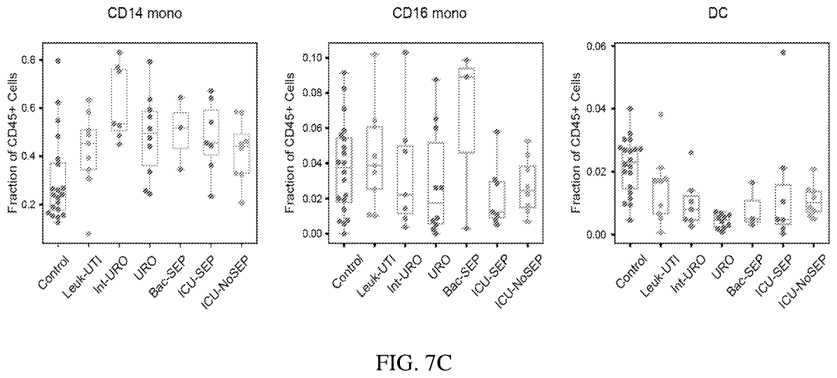

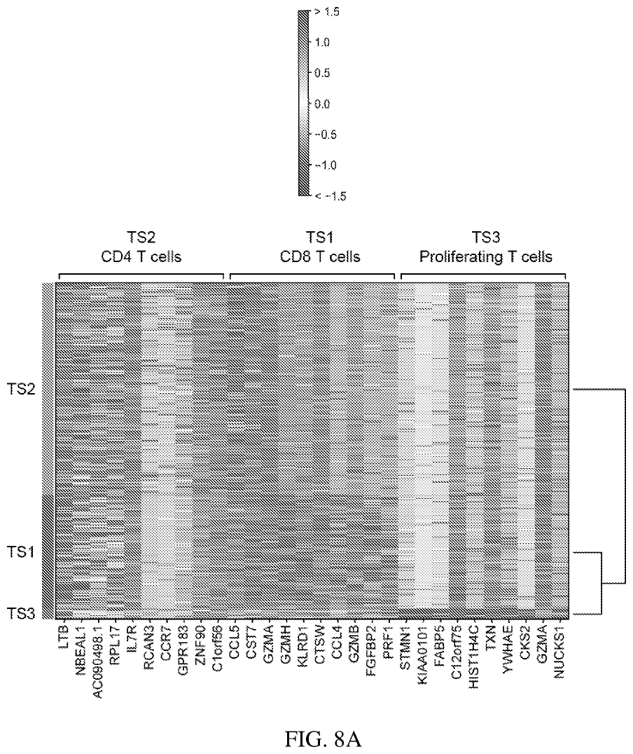

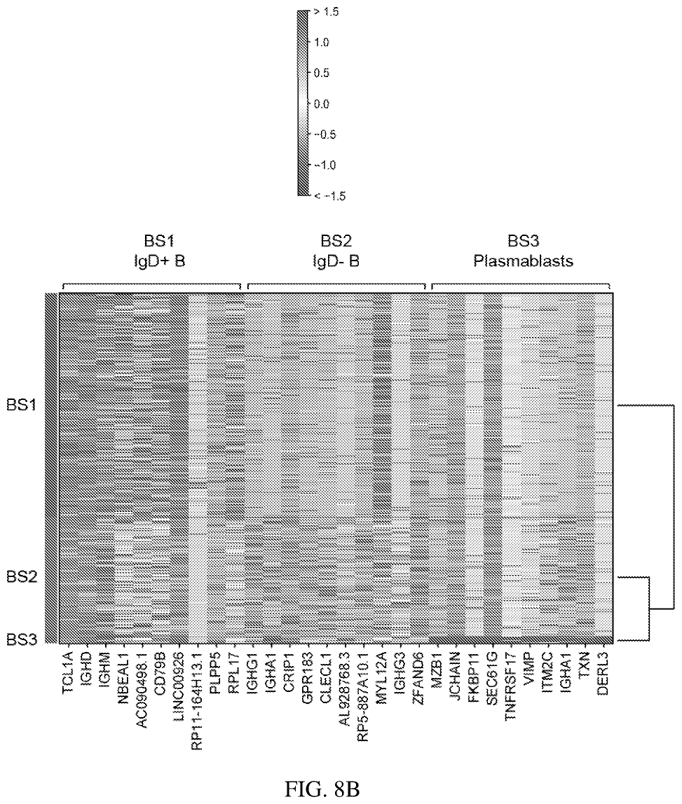

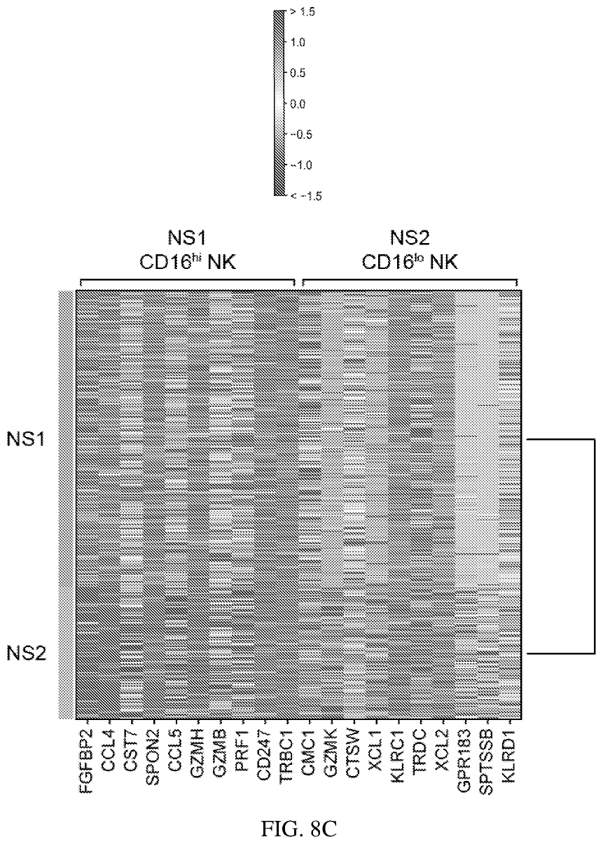

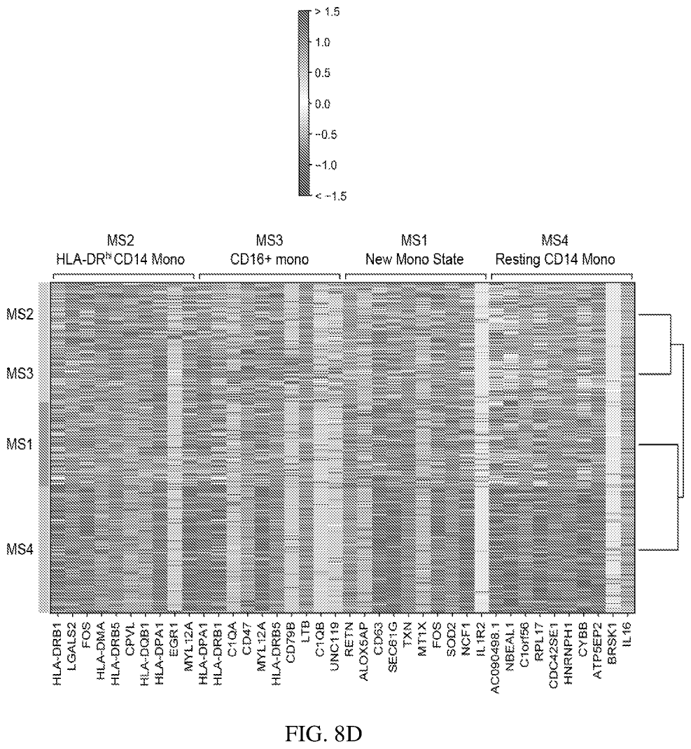

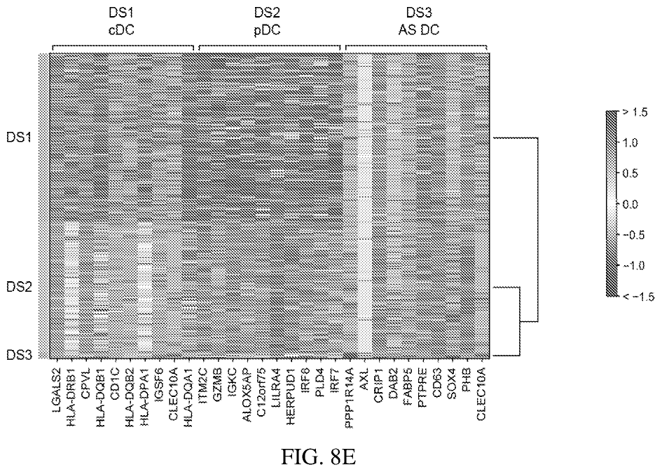

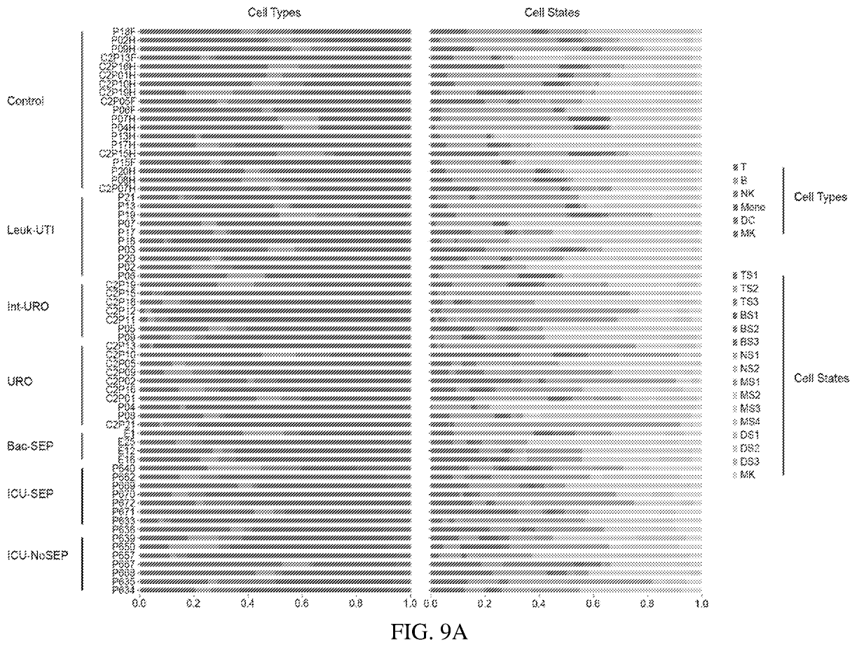

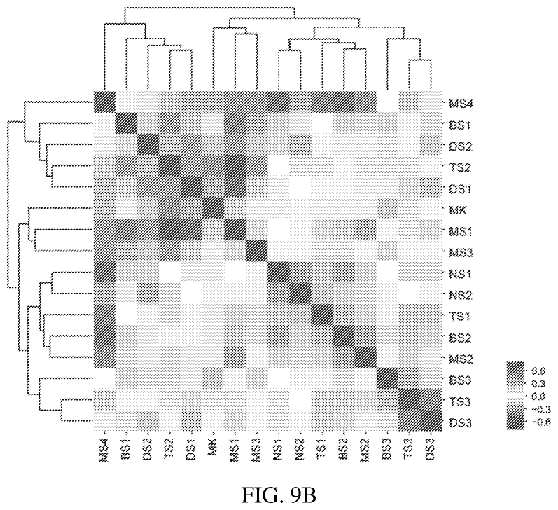

Total CD45+ PBMCs (1,000-1,500 cells per subject) and LIN-CD14-HLA-DR+ dendritic cells (300-500 cells per subject) were profiled using a 3′ tag RNA-seq approach. 6-8 samples per experiment were multiplexed using cell hashing, and observed no major batch effects in the data (FIG. 5 and Example 1). Immune-cell states by clustering the cells in two steps were identified: low-resolution clustering to identify the major immune-cell types (FIG. 1F and FIGS. 6A and 6B; FIG. 7), then subclustering each major cell type separately in a robust manner (FIG. 6C and FIG. 6D and Example 1). This approach identified cell states that were found across numerous subjects (n=31-69 per state) in different cohorts and processing batches (FIG. 2A and FIGS. 6E and 6F). Among these were transcriptional states of T, B, natural killer (NK) and dendritic cells, and importantly, four monocyte states (FIG. 8 and FIG. 9). Four distinct monocyte groups were found: (1) MS1, CD14+ cells characterized by high expression of resistin (RETN), arachidonate 5-lipoxygenase activating protein (ALOX5AP) and interleukin-1 receptor type 2 (IL1R2) (FIG. 2B); (2) MS2, characterized by high expression of class II major histocompatibility complex (MHC); (3) MS3, similar to non-classical CD16hi monocytes; and (4) MS4, which was composed of the remaining CD14+ cells that expressed low levels of both class II MHC and inflammatory cytokines. It was noted that some marker genes that characterized the MS1 state (Table 2) had been previously associated with sepsis in studies measuring either serum protein or whole-blood messenger RNA levels (Sundén-Cullberg, J. et al., Crit. Care Med. 35, 1536-1542 (2007); Lang et al., Shock 47, 119-124 (2017); Schaack et al., PLoS One 13, e0198555 (2018); and Bauer et al., EBioMedicine 6, 114-125 (2016)).

### Example 3: Expansion of a Monocyte State, MS1, in the Blood of Subjects with Sepsis

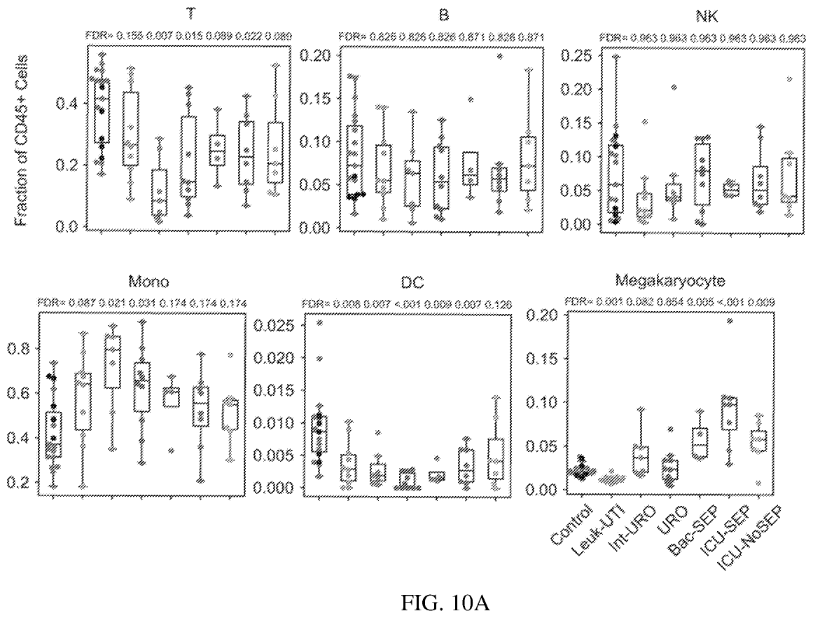

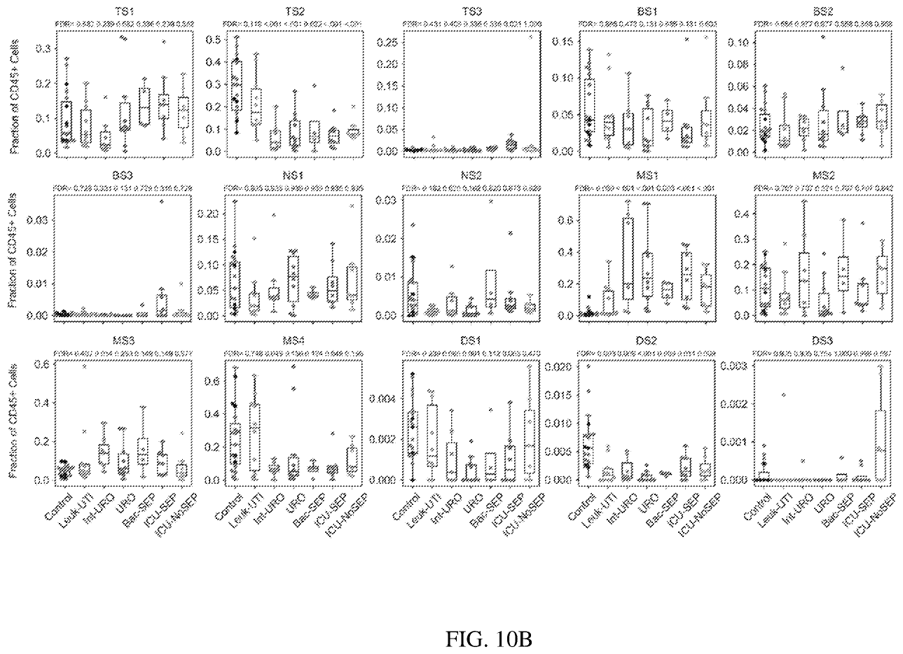

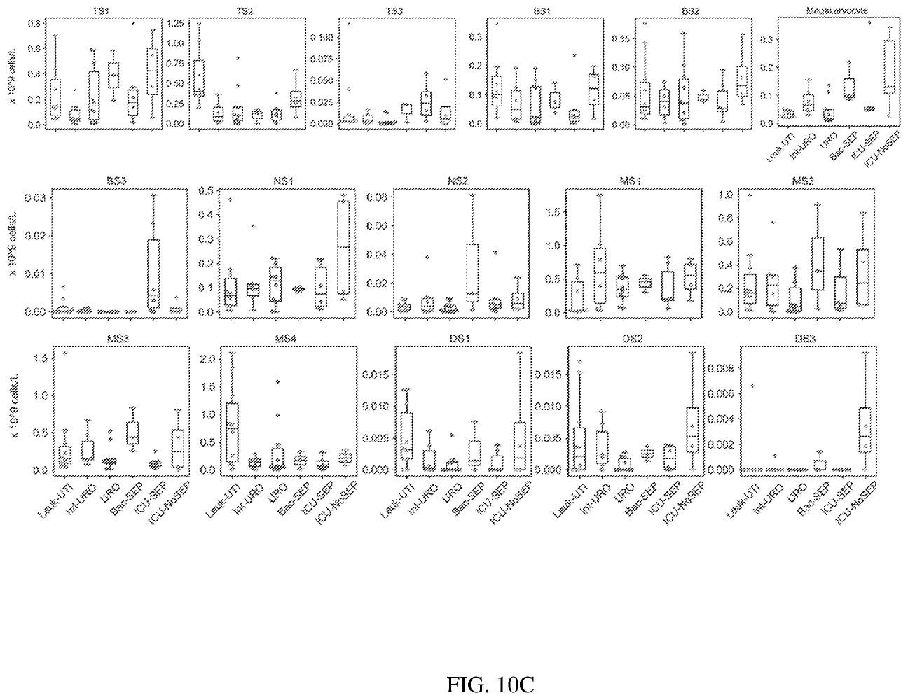

After defining clusters using data from all study subjects, the differences in abundances of cell states across different subject phenotypes was analyzed (FIG. 1F). It was found that the fractional abundances of cell states in the blood were strongly associated with the disease status of an individual (FIGS. 10A-10B), whereas absolute abundances were less so (FIG. 10C). Whereas the fractions of classical cell types vary substantially among the Control, Leuk-UTI, and sepsis (Int-URO, URO, Bac-SEP, and ICU-SEP) cohorts, more pronounced differences were found in the relative abundances of particular cell states derived from the clustering, most notably in MS1 (FIG. 2C). MS1 cells constituted a significantly larger fraction of CD45+ cells in Int-URO and URO subjects than in Control or Leuk-UTI patients (false discovery rate, FDR<0.001) and are also enriched in septic subjects in the secondary cohorts (Bac-SEP and ICU-SEP versus Control, FDR<0.001). Further, MS1 cells were present at a slightly higher fraction in septic subjects (Int-URO, URO, Bac-SEP, and ICU-SEP) than severely ill people without bacterial infection (ICU-NoSEP, FDR=0.27).

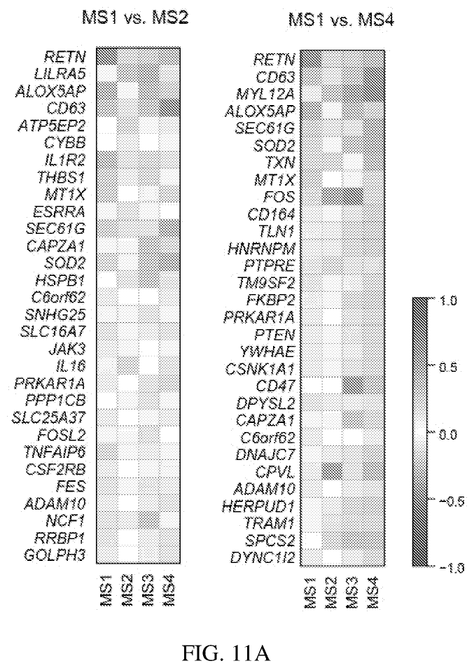

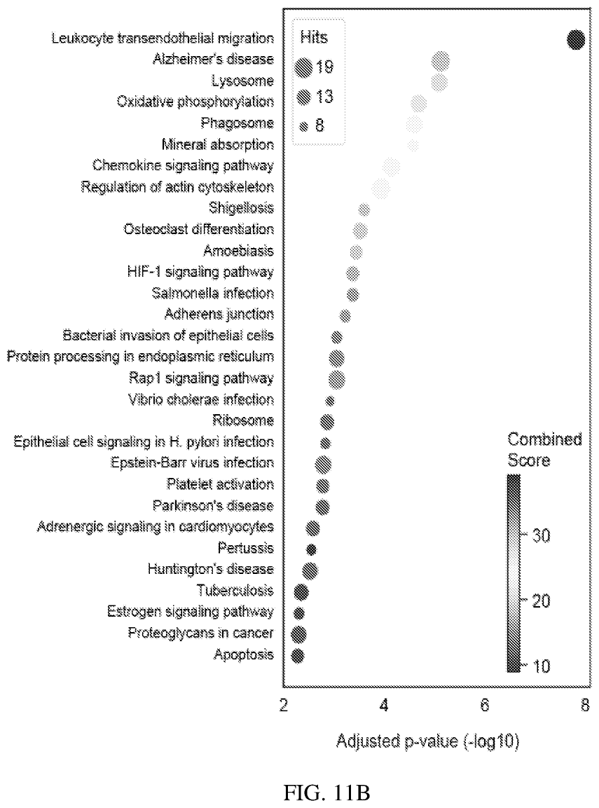

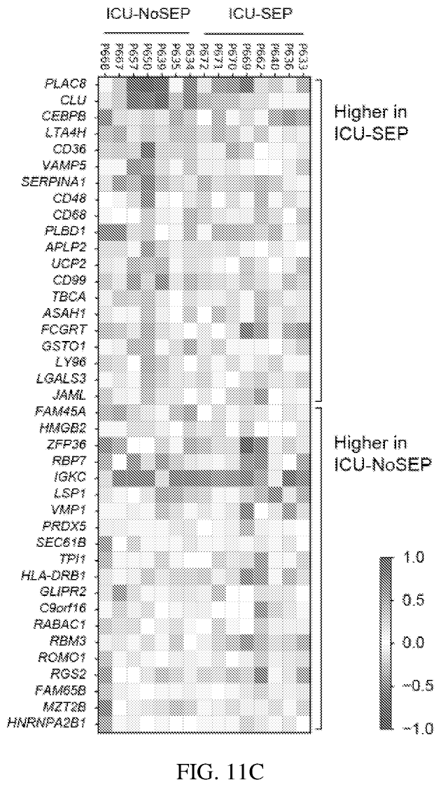

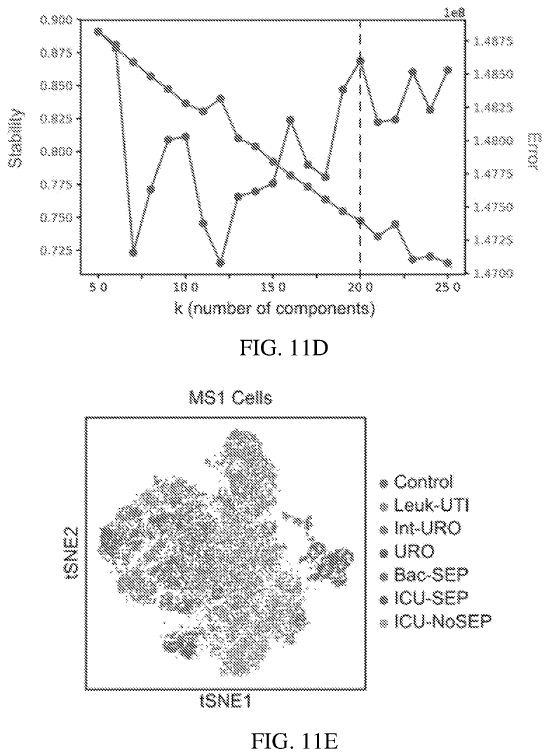

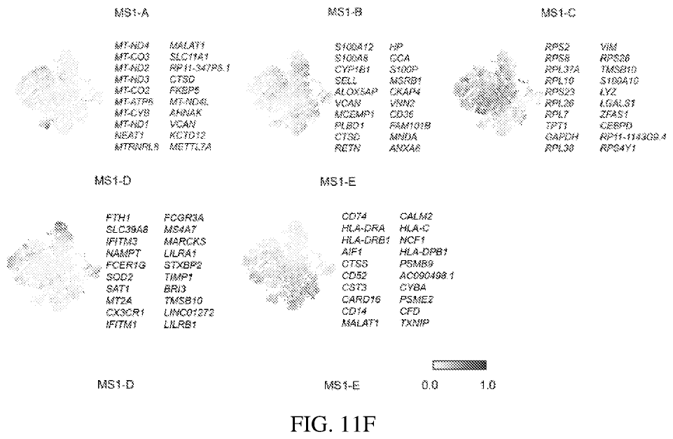

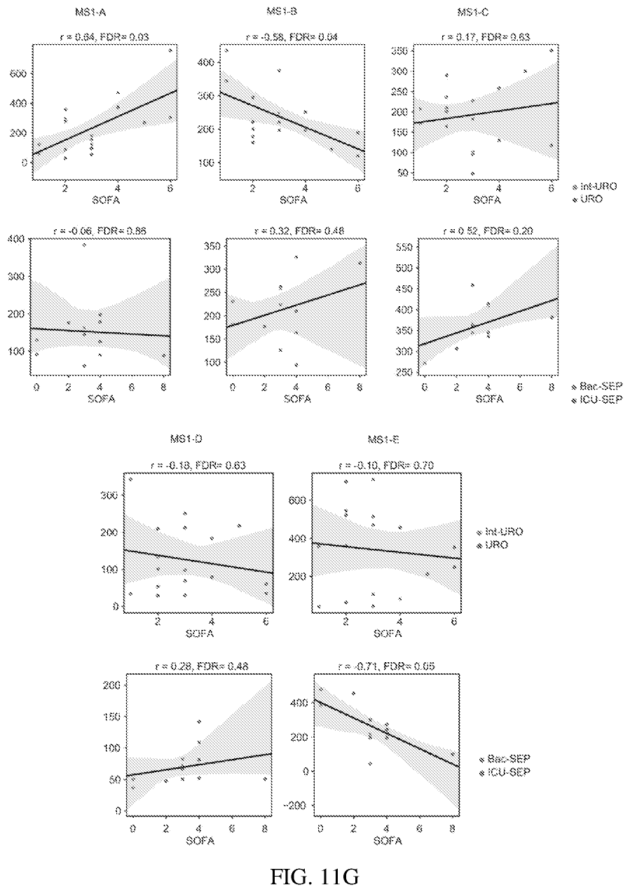

Given the expansion of MS1 in people with sepsis, it was reasoned that analysis of gene expression signatures within MS1 cells may reveal useful clinical markers for sepsis and further insight into biological mechanisms. We looked for signatures that discriminate sepsis from critical illness without bacterial infection because these cohorts were not significantly distinguished by cell-state abundance alone. Thus, genes differentially expressed in MS1 cells from ICU-SEP versus ICU-NoSEP subjects (FIG. 2D) were identified. Two genes, placenta-associated 8 (PLAC8) and clusterin (CLU), were identified that distinguished these two populations of subjects (FIG. 2E and FIG. 11). Whereas PLAC8 expression has been associated with sepsis in studies analyzing the bulk expression of blood cells, CLU expression has not, perhaps owing to its specific upregulation in MS1 cells.

Co-varying genes among MS1 cells were analyzed using non-negative matrix factorization. Five gene modules detected in more than half of the subjects with sepsis in the study were found (FIGS. 11D-11F). The module genes are disclosed in the Supplementary Table 3 in Reyes et al., An immune-cell signature of bacterial sepsis, Nature Medicine, 26, pages 333-340 (2020), which is incorporated by reference herein. Of note, the module in MS1 cells corresponding to mitochondrial respiration (MS1-A; MT-ND4, MT-CO3, MT-ATP6) correlated significantly with disease severity in subjects with sepsis from the primary cohort (Int-URO and URO, FDR=0.03; FIG. 2F), supporting the link between alterations in energy metabolism and immunoparalysis in sepsis. In addition, a module of genes in MS1 related to anti-inflammatory and pro-resolving responses (MS1-B; S100A8, RETN, ALOX5AP, FPR2) correlates negatively with severity (FDR=0.04) (FIG. 2G and FIG. 11G), consistent with a current model of sepsis wherein people early in their disease course have a heightened inflammatory state, but subsequently switch to an immunosuppressive state.

### Example 4: Validation of MS1 Signatures as Markers for Sepsis

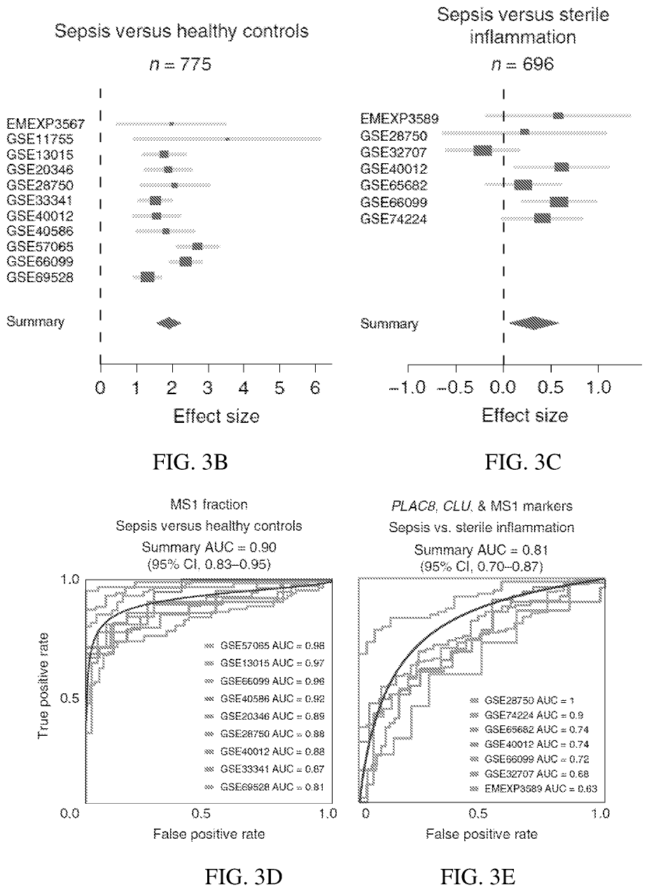

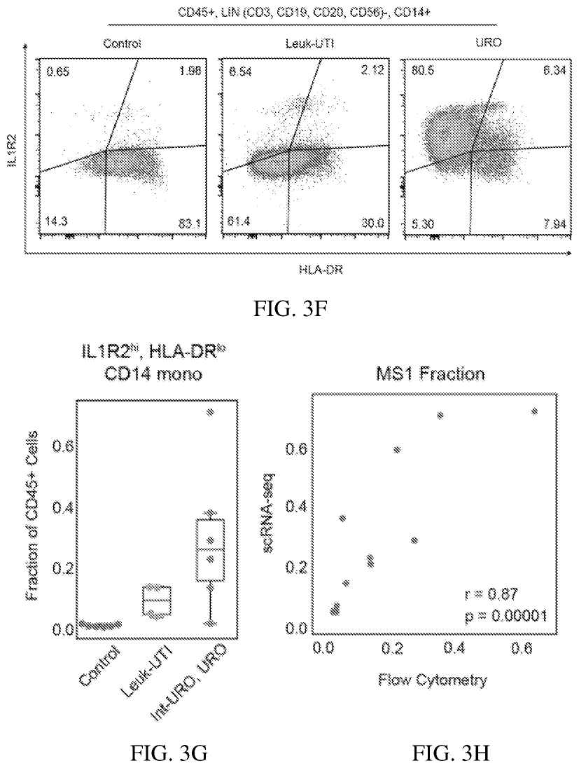

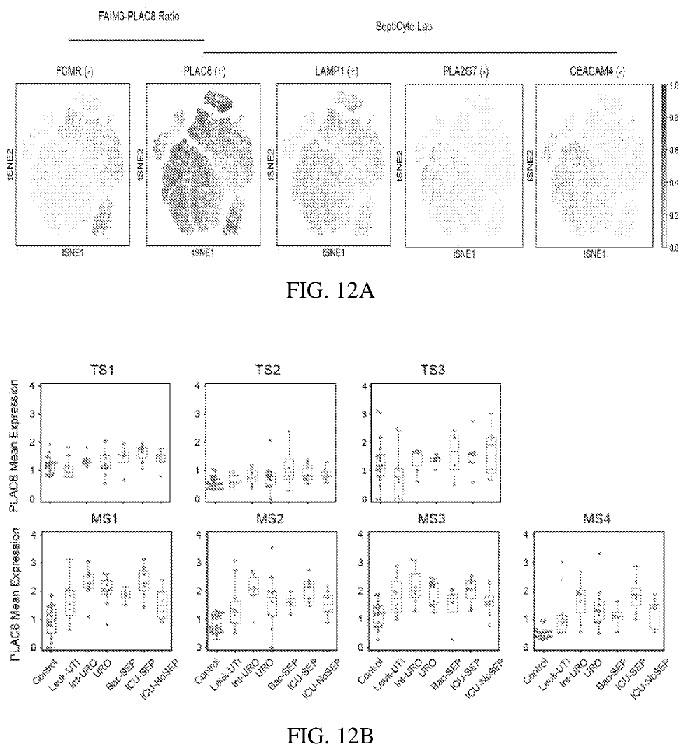

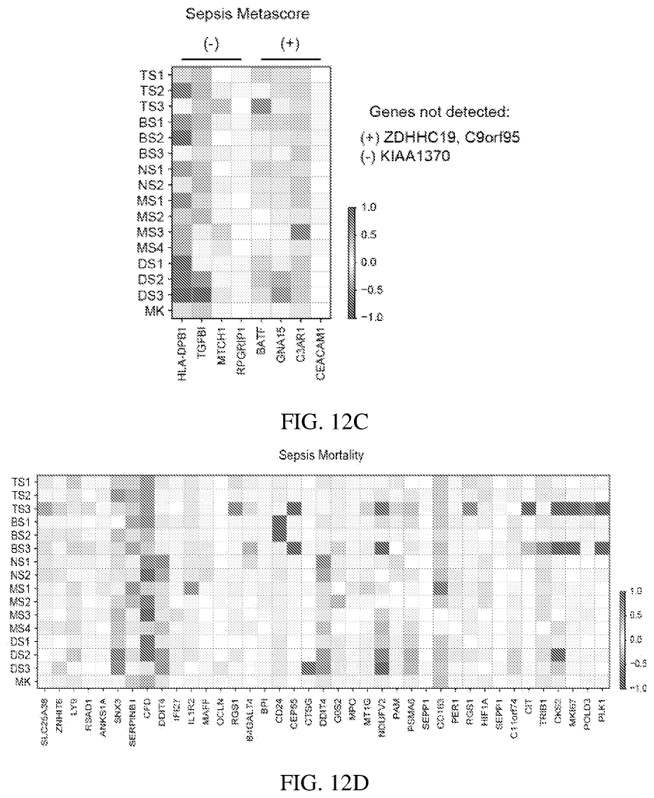

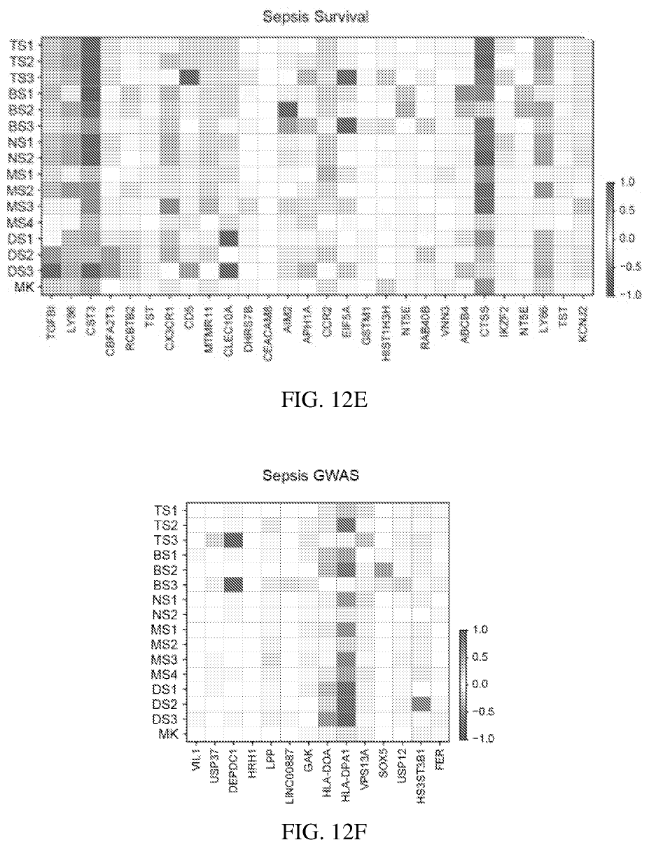

To compare the performance of the identified signatures against previously reported classifiers, we quantified the classification accuracy of the MS1 fraction, PLAC8+ CLU expression in MS1 cells, and published gene-based signatures in the cohort of the study (FIG. 3A). When classifying all individuals with sepsis (Int-URO, URO, Bac-SEP, and ICU-SEP) against Control and Leuk-UTI subjects, the MS1 fraction outperformed two published gene-set signatures (area under the curve (AUC), MS1 fraction=0.92, FAIM3/PLAC8 ratio=0.81 and SeptiCyte Lab=0.74). In addition, PLAC8+ CLU expression in MS1 cells had higher classification accuracy when comparing ICU-SEP with ICU-NoSEP subjects (AUC, MS1 PLAC8+ CLU=0.85, FAIM3/PLAC8=0.74 and SeptiCyte Lab=0.82). These external gene signatures were derived from whole-blood profiling in varying clinical contexts, which could affect their performance when applied to the PBMC-derived expression data. In addition, the performance of MS1 PLAC8+CLU may be inflated when applied to a subset of subjects from which MS1 was derived. Nevertheless, the approach provided biological context for these previously derived signature genes, as their expression in the data described in the application was specific to certain cell states (FIG. 12).

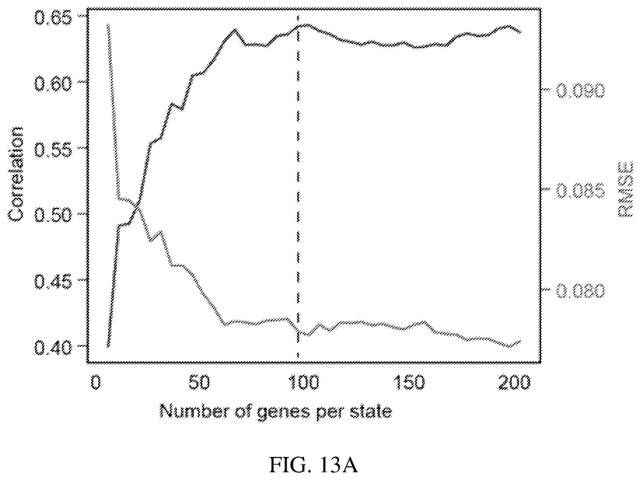

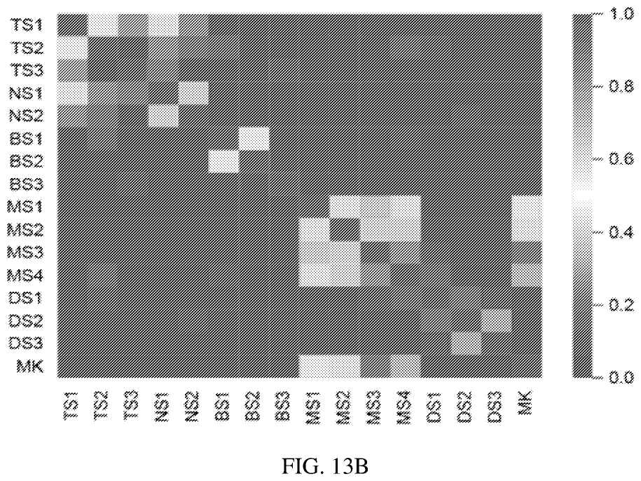

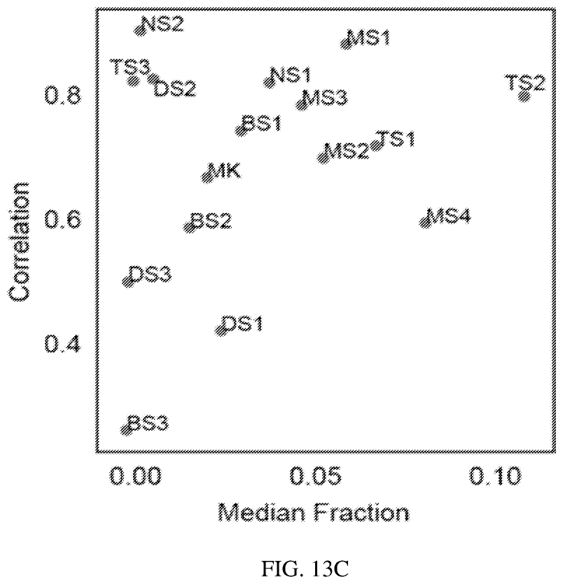

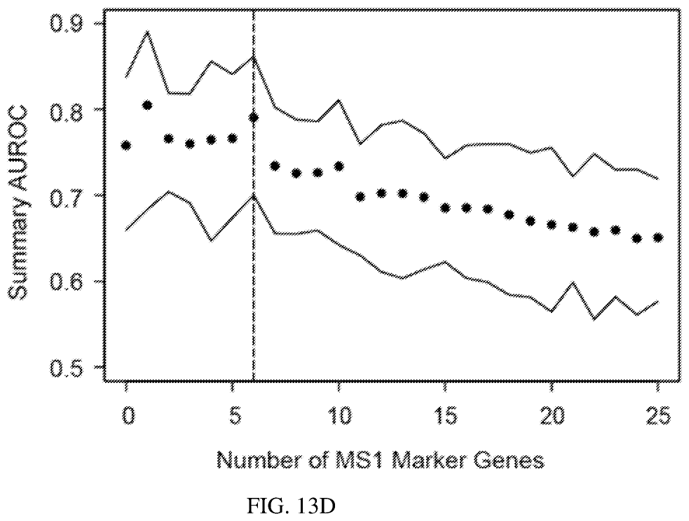

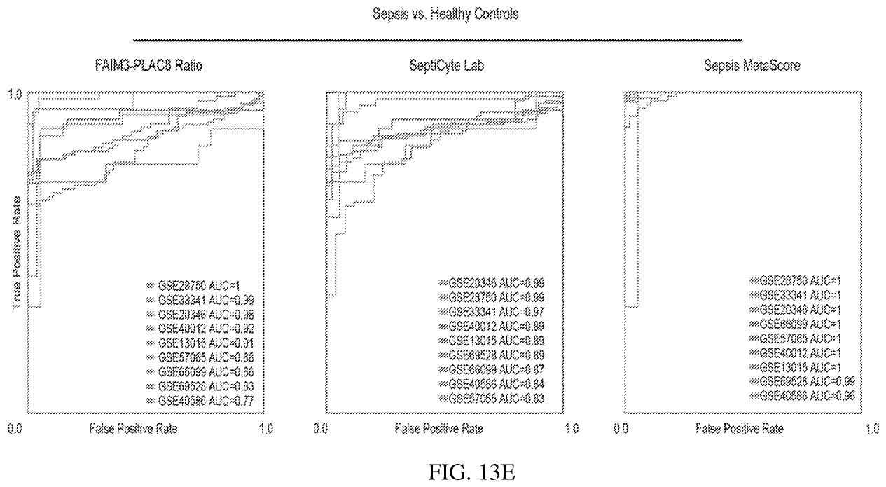

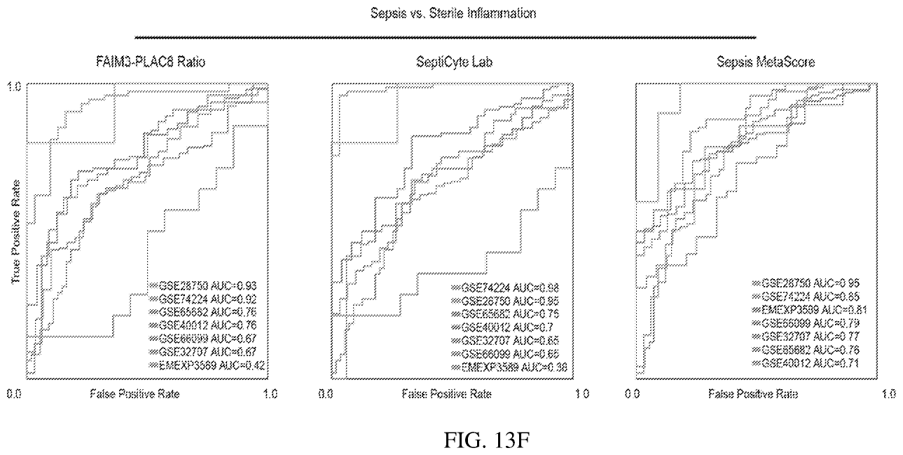

To validate the signatures in external datasets, independent cohorts of subjects with bacterial sepsis from published bulk-expression studies of sepsis were analyzed. First, the use of bulk-gene-expression deconvolution on the data was validated to infer the relative fraction of MS1 cells and cells in other states in the blood (FIGS. 13A-13C and Example 1). Upon extending this approach to bulk transcriptional data from 11 sepsis cohorts included in a recent meta-analysis, the inferred abundance of the MS1 state to be higher in people with sepsis than in controls in each study was found, with a summary effect size of 1.9 across all cohorts (FDR=1.75×10⁻³⁰, FIG. 3B and Table 3). Furthermore, the inferred MS1 fraction alone for each subject can be used as a classifier for sepsis in the same datasets, with a summary AUC of 0.90 (range of 0.81-0.98) across all studies (FIG. 3D), performing similarly to reported classifiers that were derived from bulk gene expression signatures (FIG. 13E). In a similar analysis of 7 datasets comparing people with sepsis with ICU controls (people with non-infectious systemic inflammatory response syndrome) (FIG. 3C), MS1 was expanded in sepsis, albeit with a lower but notable effect size of 0.32 (FDR=0.08), consistent with observations in the cohorts in the studies disclosed in the application. Whereas the MS1 fraction alone cannot be used as a sepsis classifier in this context, analyzing the co-expression of PLAC8, CLU, and MS1 marker genes (RETN, CD63, ALOX5AP, SEC61G, TXN, and MT1X) in these datasets performed well in classifying subjects with sepsis against sterile inflammation (FIG. 3E), with a summary AUC of 0.81 (range of 0.63-1.000.63-1.00), performing similarly to published signatures (FIGS. 13D and 13F). This analysis of published transcriptional data implied that MS1 cells were present in people with sepsis across several geographic locations, genetic backgrounds and clinical contexts, and demonstrates the potential utility of MS1-specific gene signatures for the discrimination of sepsis from sterile inflammation.

### Example 5: Surface Markers for Isolation of MS1 Cells

To improve its utility as a cytologic marker, a panel of surface proteins was identified that can be used to define the MS1 cell state by flow cytometry. Among the differentially expressed genes that distinguish it from other CD14+ monocytes, low HLA-DR and high IL1R2 expression can be used to quantify the fraction of MS1 cells (FIG. 3F). Previous studies (Gossez et al., Sci. Rep. 8, 17296 (2018); Landelle et al., Intensive Care Med. 36, 1859-1866 (2010)) showed that CD14+ monocytes from people with sepsis had decreased HLA-DR expression. However, it was found that monocytes from Leuk-UTI subjects also had this phenotype, signifying that decreased HLA-DR expression alone was insufficient to distinguish patients with sepsis from those with uncomplicated infection. By contrast, HLA-DR^{lo}IL1R2^{hi}CD14+ monocytes were at higher frequencies in Int-URO and URO subjects than in Control or Leuk-UTI subjects (FIG. 3G), and their fractions measured by flow cytometry correlated significantly with fractions determined by scRNA-seq (Pearson r=0.87) (FIG. 3H). Cells sorted with this phenotype (7,098 cells from 5 URO subjects) co-localized by expression profile with MS1 cells in the original dataset when analyzed together and projected on the same t-distributed stochastic neighbor embedding (t-SNE) plot (FIG. 3I). This combination of cell surface markers could be used to detect and/or purify the cell state for further molecular and functional characterization, or could potentially be employed as a routine monitoring tool for rapid quantification of the MS1 fraction in people at risk of sepsis.

### Example 6: Generation of MS1-Like Cells from Human Bone Marrow

Low HLA-DR expression has been associated with monocyte immaturity, resulting in decreased responsiveness to stimuli. It was hypothesized that MS1 cells might be derived from bone marrow mononuclear cells (BMMCs), which included hematopoietic precursors, rather than from mature immune cells in peripheral blood.

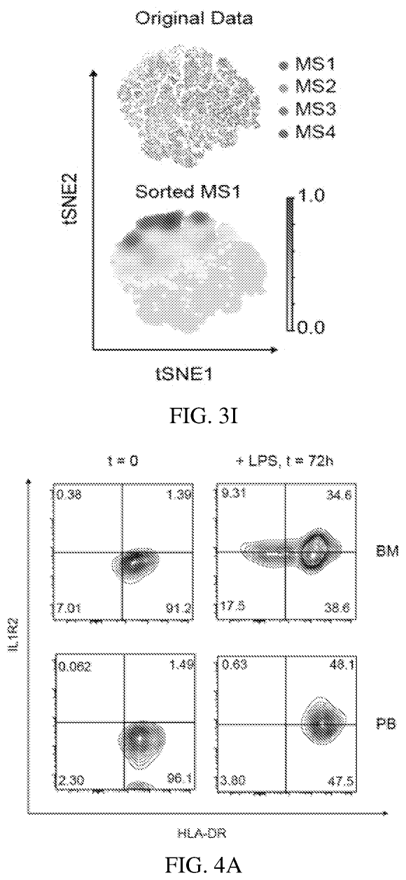

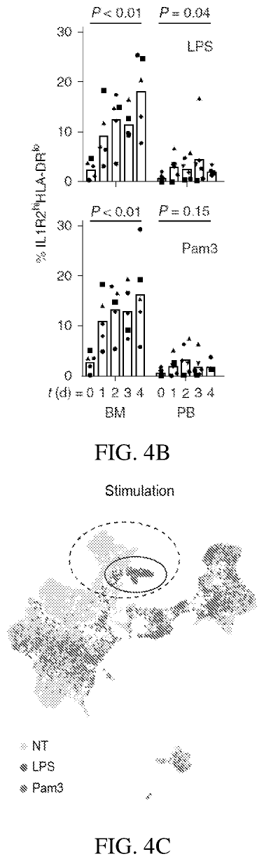

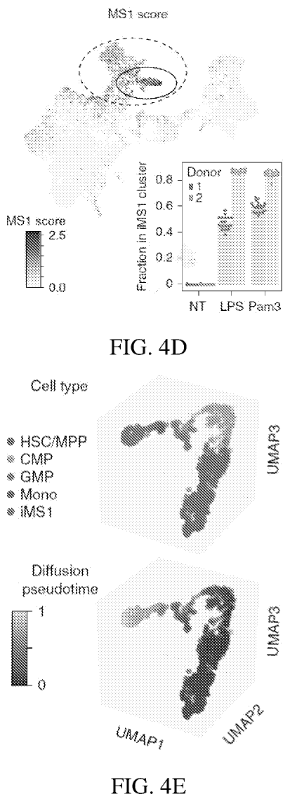

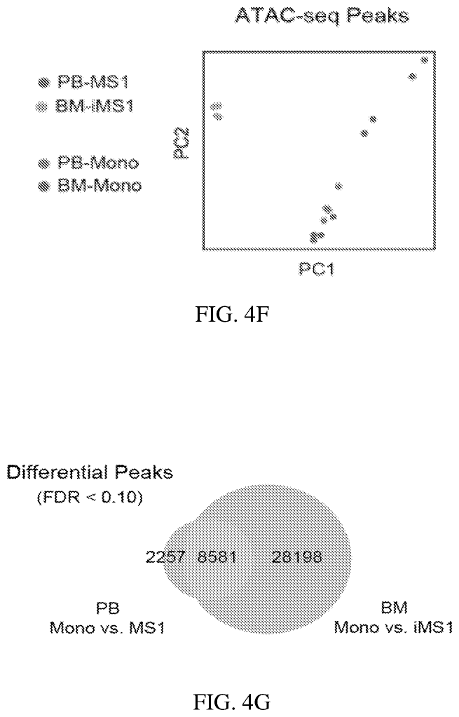

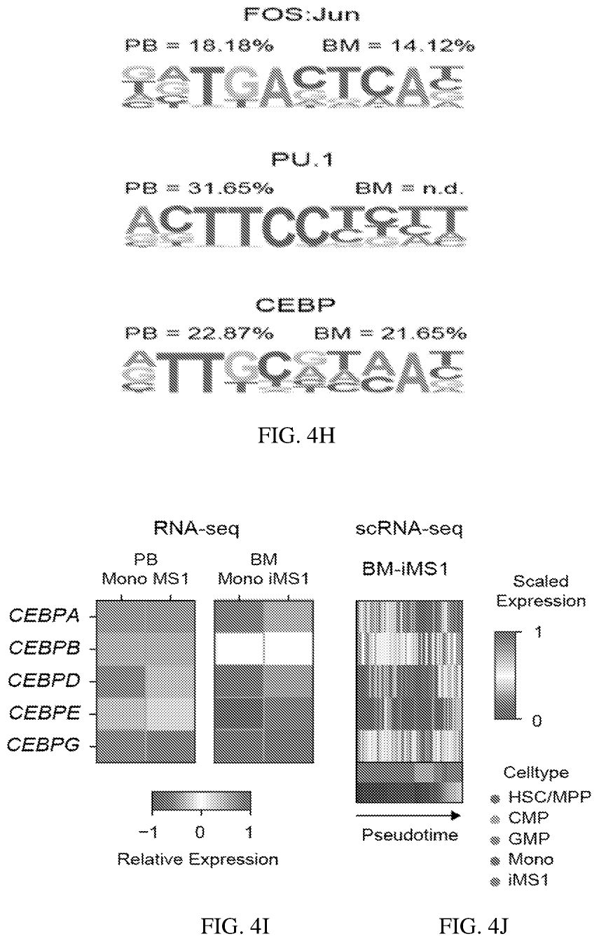

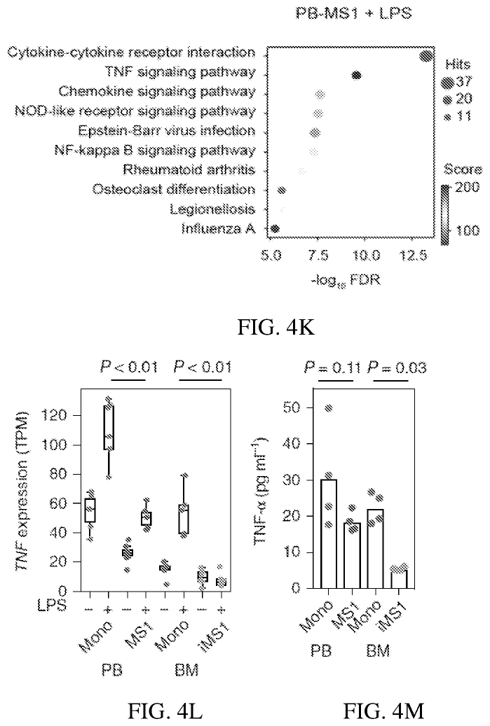

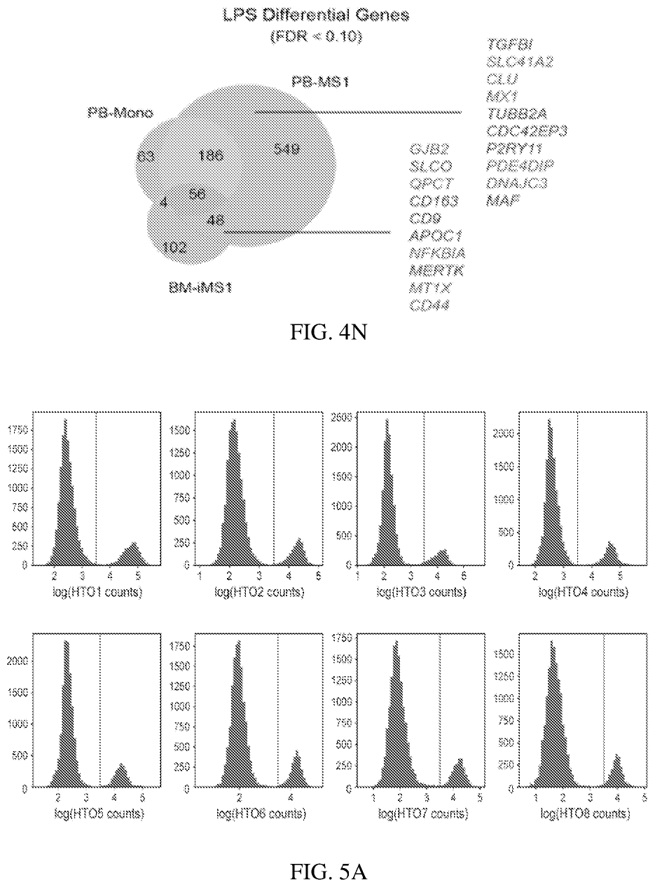

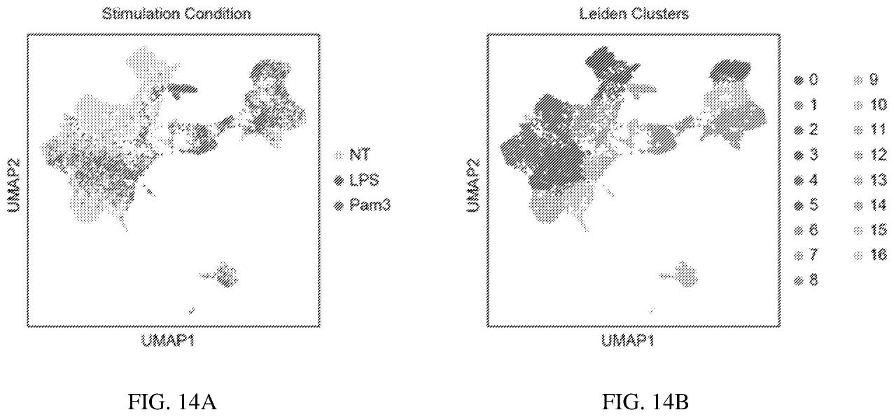

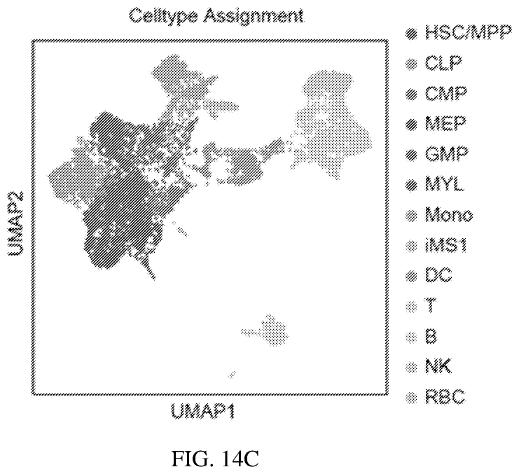

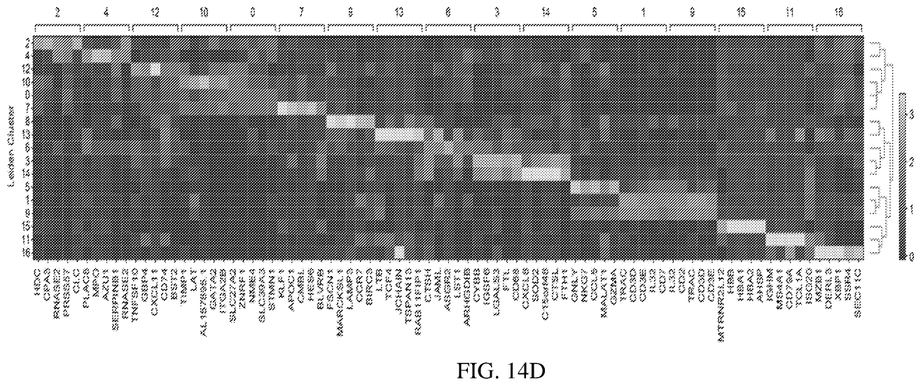

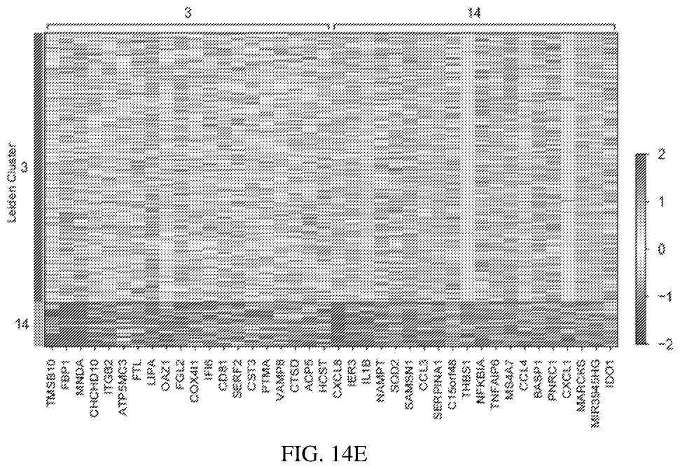

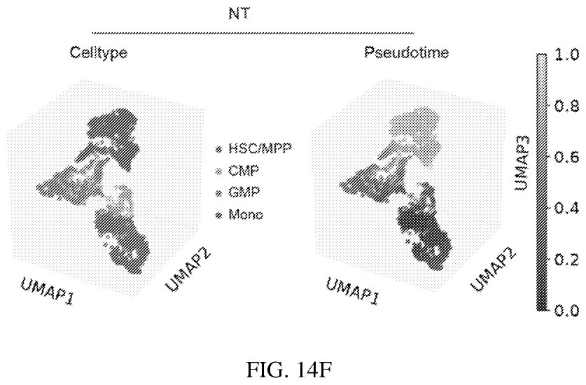

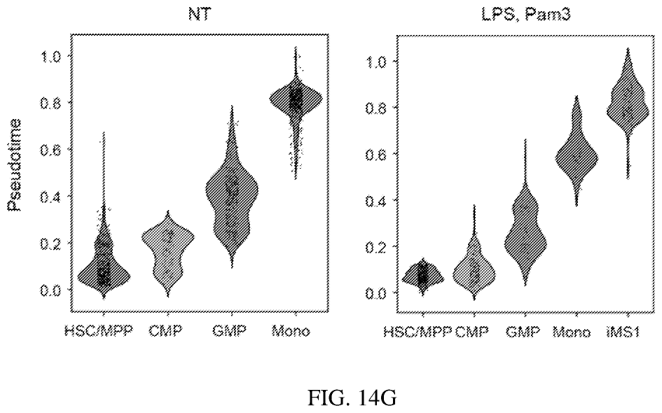

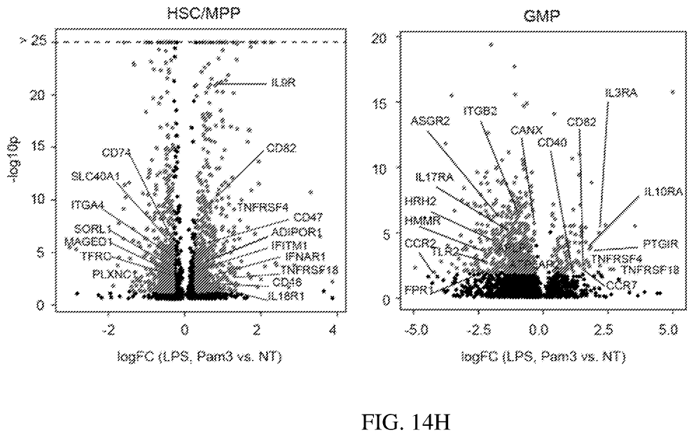

It was found that chronic stimulation of BMMCs with Pam3CSK4 (Pam3) or lipopolysaccharide (LPS) resulted in the emergence of a HLA-DR^{lo}IL1R2^{hi}CD14+ population (FIG. 4A). The abundance of this population, as a fraction of total CD14+ cells, increased significantly over time in treated BMMCs, but not in treated PBMCs (FIG. 4B). Furthermore, scRNA-seq profiling of BMMCs treated with LPS or Pam3CSK4 revealed a cluster of cells scoring highly for MS1 signature genes that were absent in the untreated condition (FIGS. 4C-4D and FIG. 14A-14E). Trajectory analysis of the myeloid populations suggested that the MS1-like induced population (iMS1, Leiden cluster 14) proceeded initially through a differentiation pathway similar to that of cells from the non-stimulated condition, but that it subsequently deviated from this fate (FIG. 4E and FIGS. 14F-14G). Progenitor populations in the stimulated condition displayed several differentially expressed genes (FIG. 14H). Stimulated progenitor cells upregulated several receptors previously associated with inflammation-induced myelopoiesis (for example, IL3R, IL10R, IFNAR1), suggesting that an MS1-like population may emerge in the bloodstream as a result of sepsis-induced myelopoiesis. These results demonstrated the potential of human bone marrow cells as a model for the expansion of the MS1 state in sepsis, and supported the hypothesis that the emergence of reprogrammed myeloid cells in the blood stems from dysregulated differentiation of hematopoietic precursors.

### Example 7: Epigenomic Landscape and Transcriptional Regulators of MS1 Cells

The chromatin accessibility landscapes of monocytes from peripheral blood of healthy controls (PB-Mono), MS1 cells sorted from patients with sepsis (PB-MS1), monocytes from healthy bone marrow (BM-Mono), and monocytes from BMMCs stimulated with LPS and HSC cytokines (BM-iMS1) were profiled. Principal component analysis of genome-wide ATAC-seq profiles of the four populations showed that PB-Mono and BM-Mono co-localized, whereas PB-MS1 and BM-iMS1 formed distinct clusters yet shared similar loadings on PC2 (FIG. 4F). Motif enrichment analysis on the differential peaks between PB-Mono and PB-MS1 demonstrated enrichment of the FOS-Jun, PU.1 and CEBP motifs, all of which were families of transcription factors critical to monocyte development (FIGS. 4G and 4H). The MS1 peaks were disclosed in the Supplementary Table 5 in Reyes et al., An immune-cell signature of bacterial sepsis, Nature Medicine, 26, pages 333-340 (2020), which is incorporated by reference herein in its entirety. Given their important role in inflammation-induced myelopoiesis, the expression of the CEBP transcription factors was analyzed. Bulk RNA-seq showed an increase in CEBPD (CEBPδ) and CEBPE and a decrease in CEBPG expression in PB-MS1 compared with PB-Mono, and similarly in BM-iMS1 compared with BM-Mono (FIG. 4I). Analysis of the differentiation trajectory of iMS1 cells from bone-marrow progenitors also showed an increase in CEBPD expression after the transition from a GMP state (FIG. 4J). Interestingly, CEBPD was among the top genes of the module comprised of transcription-related and housekeeping genes from the analysis of MS1 cells from people with sepsis (MS1-C, FIG. 11F), suggesting its potential importance in the maintenance of the MS1 program. The module genes were disclosed in the Supplementary Table 3 in Reyes et al., An immune-cell signature of bacterial sepsis, Nature Medicine, 26, pages 333-340 (2020), which is incorporated by reference herein in its entirety. Altogether, these analyses showed that MS1 cells had an epigenomic profile markedly different from that of normal CD14+ blood monocytes, and that these differences were associated with transcription factors involved in monocyte differentiation. Although in vitro-generated BM-iMS1 did not fully recapitulate the epigenomic landscape of MS1 cells, the two populations showed significant overlap in accessible peaks and shared the upregulation of similar transcriptional regulators.

### Example 8: Functional Response of MS1 Cells to Restimulation

To compare the functional responses of MS1 cells to those of other CD14+ monocytes, the four monocyte populations' cells were sorted and stimulated with 100 ng ml⁻¹ LPS after resting for 24 hours. As expected, LPS stimulation resulted in upregulation of genes related to cytokine secretion and activation of the nuclear factor-κB (NF-κB) signaling pathway (FIG. 4K). The LPS response differential expression was disclosed in the Supplementary Table 6 in Reyes et al., An immune-cell signature of bacterial sepsis, Nature Medicine, 26, pages 333-340 (2020), which is incorporated by reference herein in its entirety. However, the magnitude of response was decreased in PB-MS1 cells relative to PB-Mono, and in BM-iMS1 relative to BM-Mono, as evidenced by lower basal and induced expression of the tumor necrosis factor (TNF) gene, and less secretion of the TNF-α protein (FIG. 4L-4M). Analyzing the overlap in differentially expressed genes upon stimulation revealed a large number of genes that were uniquely upregulated in PB-MS1 (FIG. 4N). This included CLU, one of the genes proposed as a marker for discriminating sepsis from noninfectious inflammation. BM-iMS1 also up-regulated a subset of the genes (12.4%) induced in PB-MS1. Of note, NFKBIA, a known inhibitor of inflammatory responses, was upregulated in both PB-MS1 and BM-iMS1, perhaps explaining the blunted response in both populations. This analysis demonstrated that MS1 cells from people with sepsis and those induced from human bone marrow both have a dysregulated response to further bacterial stimulation, recapitulating known phenotypes of monocytes in people with sepsis.

### Example 9: Characterization of the Gene Expression Module of MS1 Incubated with Bone Marrow Progenitor

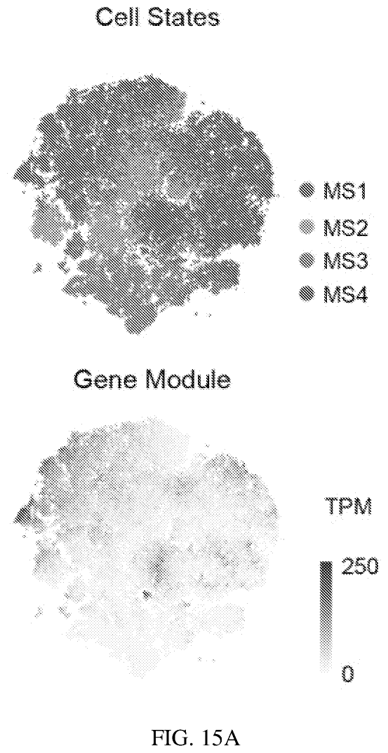

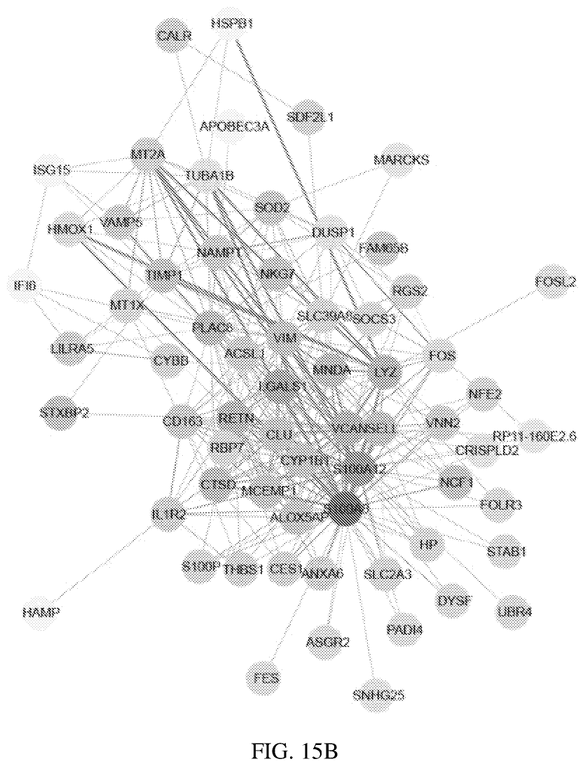

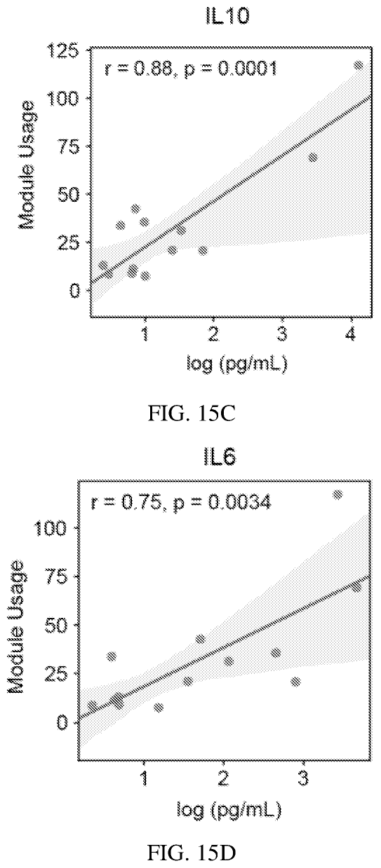

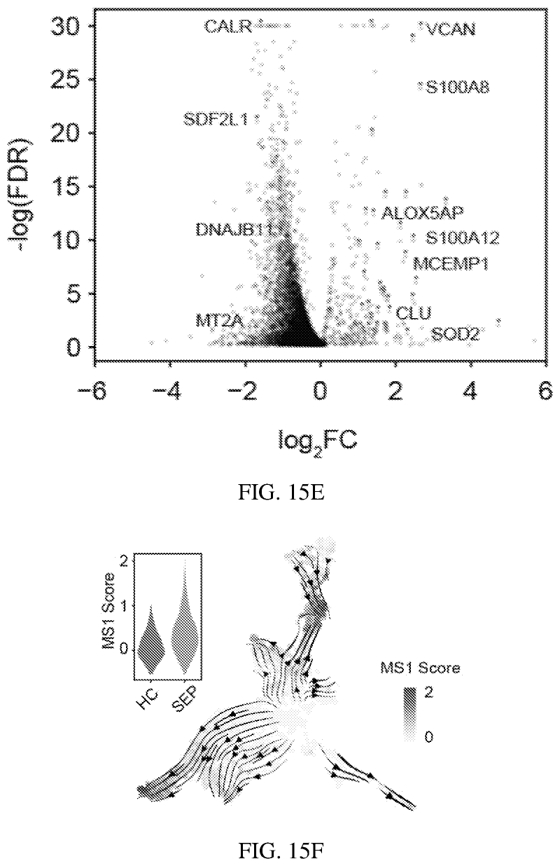

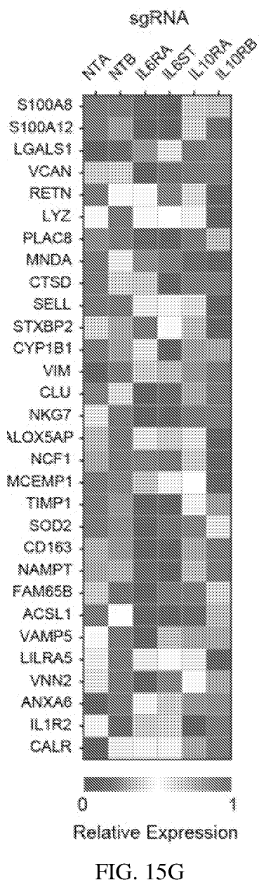

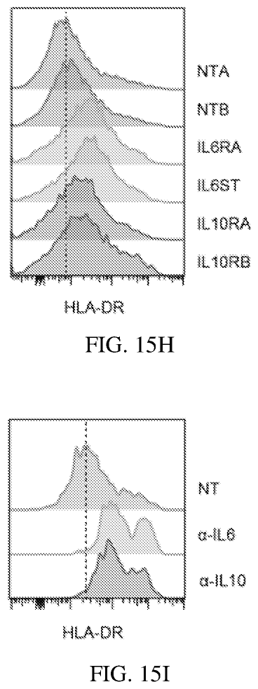

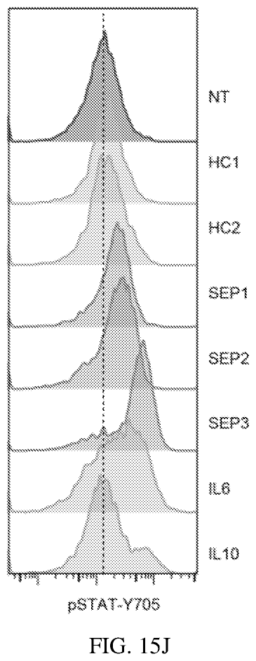

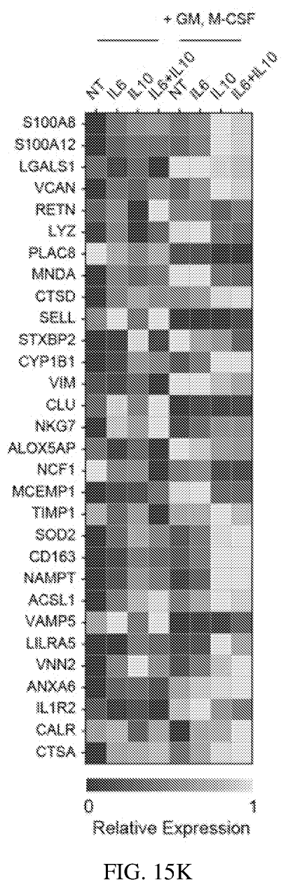

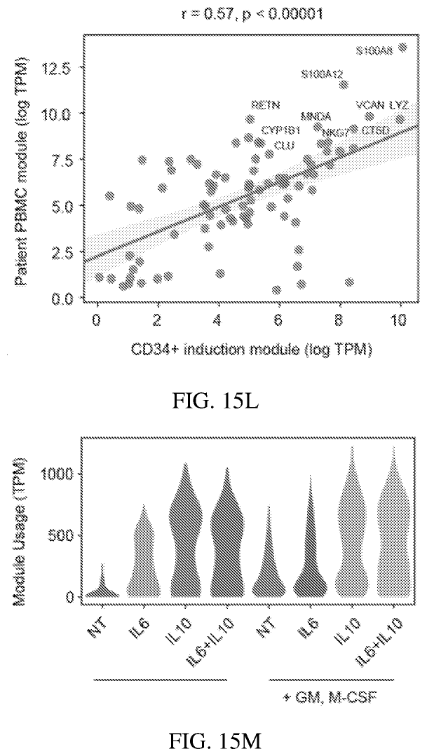

To evaluate the gene expression signature of MS1 cells, non-negative matrix factorization was performed. The original cell states visualized with t-SNE projection versus module usage of MS1, Ms2, MS3, and MS4 are shown in FIG. 15A and FIG. 15B. The top graph of FIG. 15A showed original classification of cells from the cohorts. Gene module was expressed as TPM (transcript per million). To examine whether cytokines promoted the growth and preparation of MS1 cells, IL6 and IL10 were incubated with MS1 cells. As shown in FIG. 15C and FIG. 15D, the usage of the MS1 module correlated with IL6 and IL10 plasma levels.

To evaluate the effects of bone marrow progenitor cells on the growth and production of MS1 cells, CD34+ hematopoietic stem & progenitor cells (HSPCs) were co-incubated with sepsis plasma (20%) or healthy plasma (e.g. without sepsis) for 7 days. FIG. 15E showed that CD34+ HSPCs produced monocytes with higher expression of MS1 genes compared with the healthy plasma counterparts. Differential gene expression was conducted to further evaluate MS1 gene signature. IL6 or IL10 receptors were knocked out by using a CRISPR guide RNA to further determine the effects of IL6 and IL10 on CD34+ HSPCs-incubated MS1 cells. As shown in FIG. 15G, the incubation in sepsis plasma of HSPCs with IL6 or IL10 receptors knocked out demonstrated reduction in expression of MS1 genes (e.g. S100A8 and MNDA) compared with the no treatment groups (NTA and NTB). Similarly, differential gene expression was conducted to evaluate MS1 gene signature incubated with IL6, IL10, or IL6 and IL10 in the presence or absence of GM-CSF (FIG. 15K). In general, at least S100A8, S100A12, VCAN, RETN, LYZ, MNDA, CTSD, SELL, CYP1B1, CLU, NKG7, MCEMP1, TIMP1, SOD2, CD163, NAMPT, ACSL1, VAMP5, LILRA5, VNN2, ANXA6, CALR, and CTSA were upregulated with the incubation of IL6 and/or IL10, especially in the presence of GM-CSF.

Incubation in sepsis plasma of HSPCs with IL6 or IL10 receptors knocked out showed partial rescue of HLA-DR expression (FIG. 15H and FIG. 15I). HLA-DR is an MHC class II cell surface receptor encoded by the human leukocyte antigen complex. As known in the art, the primary function of HLA-DR is to present peptide antigens, potentially foreign in origin, to the immune system, thereby regulating T cell response, for example. FIG. 15J showed that the incubation of HSPCs in sepsis plasma resulted in STAT3-Y705 phosphorylation, which represents a downstream target of both IL6 and IL10 signaling. Further, the MS1 module derived de novo from CD34+ HSPCs differentiated with cytokines as described in the present disclosure was compared with the expression from sepsis patients' PBMCs (FIG. 15L). The usage of the MS1 module differentiated across different cytokine conditions were also examined (FIG. 15 M): (1) NT: no treatment, (2) IL6 only, (3) IL10 only, and (4) IL6 and IL10 in the presence or absence of GM-CSF and/or M-CSF. IL10 resulted in higher module usage (TPM), regardless of the presence or absence of GM-CSF and/or M-CSF.

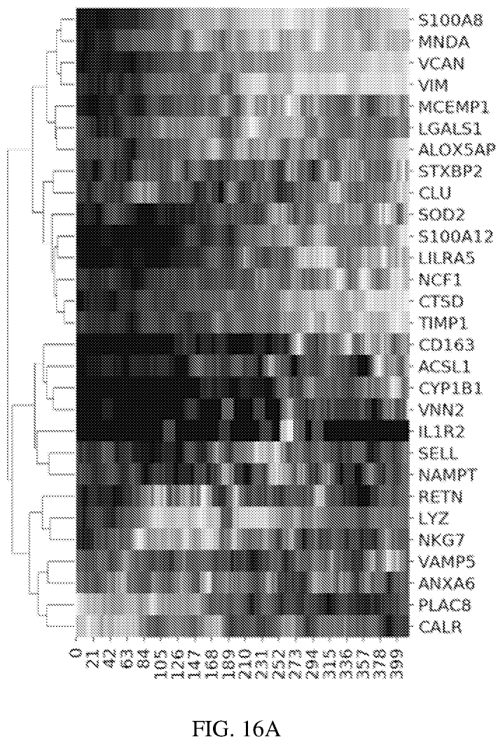

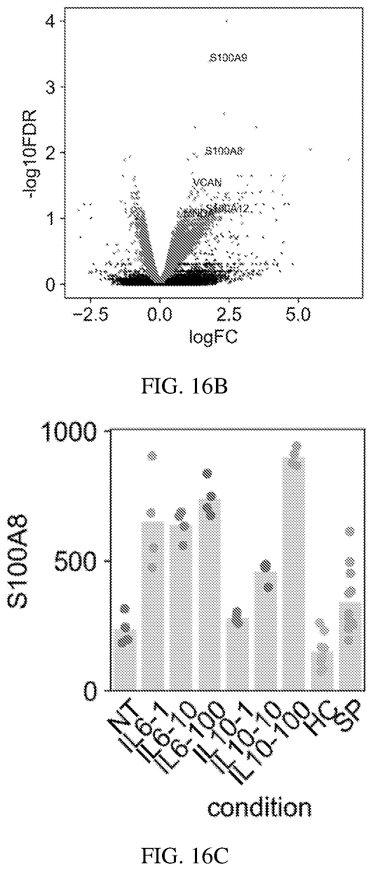

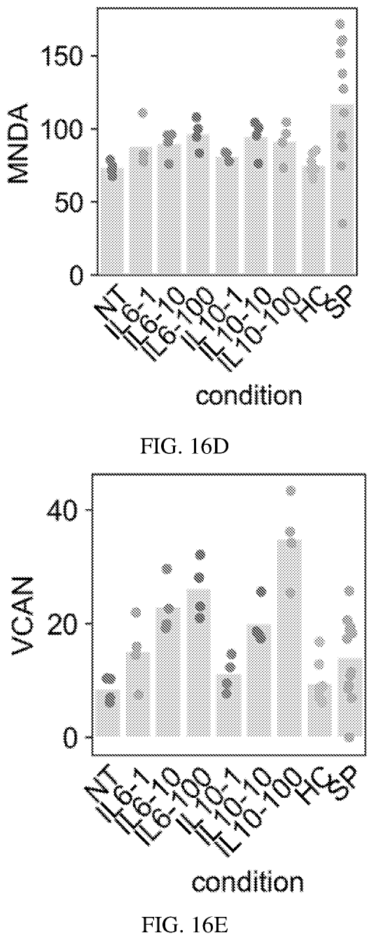

To analyze genes along the trajectory pathway from HSPCs to MS1-like monocytes, a differential gene expression assay was performed. As shown in FIGS. 16A-FIG. 16C, at least S100A8, MNDA, and VCAN gene expression was up-regulated after 24 hour incubation. These genes further remained up-regulated throughout the tested time points.

### Example 10: The Effect of MS1 Cells on T Cell Proliferation

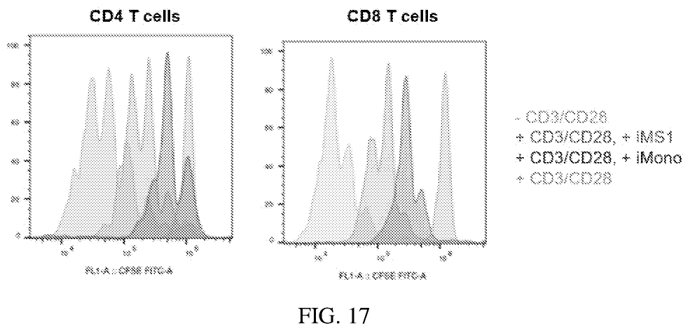

To assess the effect of MS1 cells (iMS1) on T cell proliferation, CD4 T cells and CD8 T cells were co-incubated with the following conditions: (1) no treatment, (2) CD3/CD28 T cell activator, (3) CD3/CD28 T cell activator+MS1 cells (iMS1), or (4) CD3/CD28 T cell activator+iMono cells. The MS1 cells used were derived from a different donor than the donor of CD4 T cells and CD8 T cells. After the treatments, CFSE (carboxyfluorescein succinimidyl ester) cell proliferation analysis by flow cytometry was performed, using a protocol known in the art. As shown in FIG. 17, both CD4 T cells and CD8 T cells were activated and proliferated by the CD3/CD28 T cell activator at earlier time points when the assay was performed. The addition of the MS1 cells delayed such proliferation of both CD4 T cells and CD8 T cells. Co-incubation with the MS1 cells also suppressed the proliferation of both CD4 T cells and CD8 T cells. This analysis demonstrated that MS1 cells were able to delay and/or inhibit the proliferation of T cells. These results suggested that MS1 cells as disclosed in the present disclosure could be used as an immunosuppressive treatment for regulating T cell populations.

### Example 11: Gene Profiling of Renal Epithelial Cells Co-Incubated with MS1 Cells

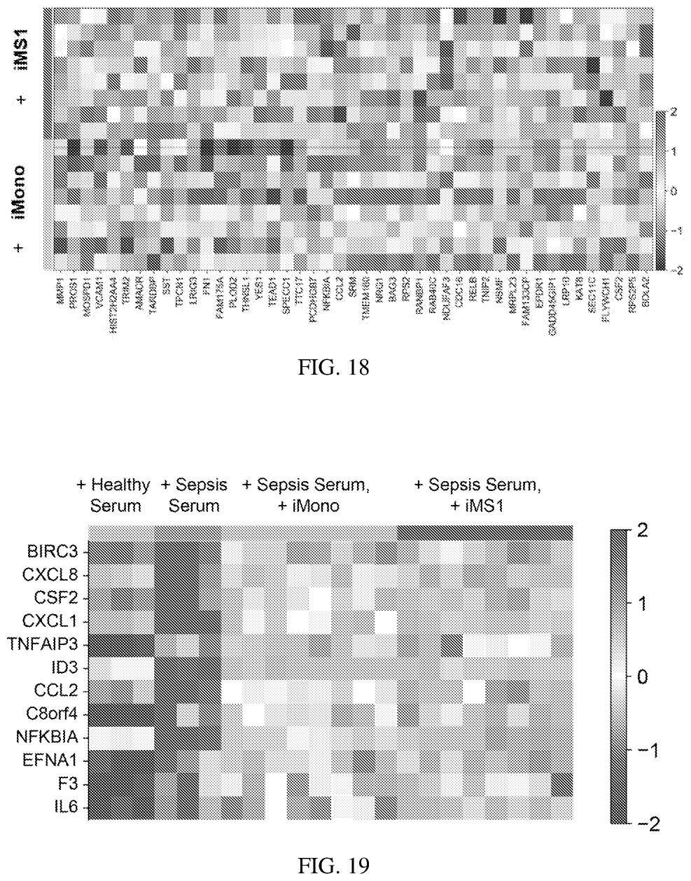

To characterize the effects of MS1 cells on differential gene expression, renal epithelial cells were incubated with either MS1 cells or iMono cells for at least 24 hours before RNA sequencing analysis was performed. The heatmap graph in FIG. 18 shows that gene signatures of renal epithelial cells incubated with MS1 cells were generally opposite from the renal epithelial cells incubated with iMono cells. For example, MMP1 (collagenase), PROS1 (protein S, regulates clotting), VCAM1 (adhesion molecule), SST (somatostatin, pleiotropic hormone, decreases renal blood flow), and FN1 (fibronectin) were upregulated by MS1 cells (iMS1) cells, while down-regulated by the iMono cells.

To further examine the effects of sepsis serum on inflammatory cytokine expression in the renal epithelial cells in the presence or absence of MS1 cells, the renal epithelial cells were categorized to the following treatment groups: (1) healthy serum, (2) sepsis serum, (3) sepsis serum+MS1 cells, or (4) sepsis serum+iMono cells. As shown in FIG. 19, as expected, healthy serum did not induce inflammatory cytokine expression, whereas sepsis serum upregulated inflammatory cytokine expression (e.g. BIRC3, CXCL1, CSF2). When MS1 cells were added, the inflammatory cytokine expression upregulated by sepsis serum was substantially reduced compared with iMono cells. For instance, CXCL1 was suppressed with the MS1 cell treatment to levels that were similar to healthy serum. Taken together, this analysis showed that MS1 cells regulated the activated renal epithelial cells by reducing their expression of inflammatory cytokines.

### Example 12: Characterization of Gene Signatures of Endothelial Cells with MS1 Cell Treatment

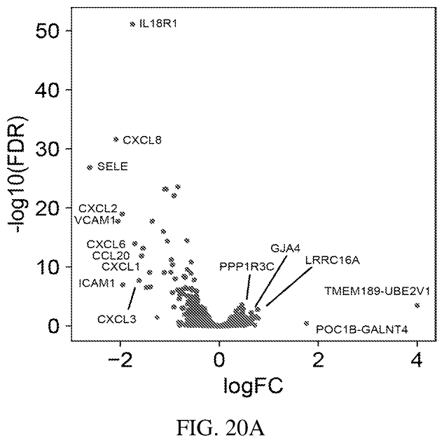

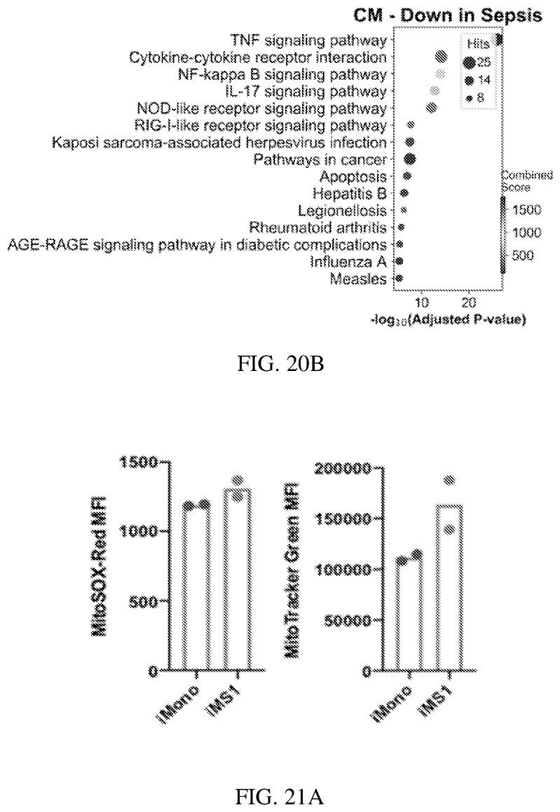

To determine the role of MS1 cells on endothelial cells, conditioned media from MS1 as described in the present disclosure was used for incubating endothelial cells. Differential expression analysis was performed and the results were conducted by two-sided Wilcoxon rank-sum test. As shown in FIG. 20A, several chemokine associated genes such as CXCL6, CCL20, and CXCL1 were suppressed in endothelial cells in the presence of conditioned media from MS1. To further characterize gene signatures, enrichment of pathways (KEGG database) for downregulated genes in MS1 cells were conducted. As shown in FIG. 20B, the largest size of circles, which also corresponded to the number of gene hits in a set (i.e. hits=25), represented cytokine-cytokine receptor interaction and pathways in cancer. Among these two pathways, cytokine-cytokine receptor interaction had higher enrichment score. The second largest size of circles (i.e. hits at least=14), represented pathways such as NF-kB signaling pathway, IL-17 signaling pathway, NOD-like receptor signaling pathway, Kaposi sarcoma-associated herpesvirus infection, apoptosis, hepatitis B, influenza A, and measles. Among these pathways, NF-kB signaling pathway, and IL-17 signaling pathway had the highest enrichment scores. Taken together, the results demonstrated that the addition of MS1 cells to activated endothelial cells reduced their expression of adhesion molecules and chemokines.

### Example 13: Characterization of the Phenotype of MS1 Cells

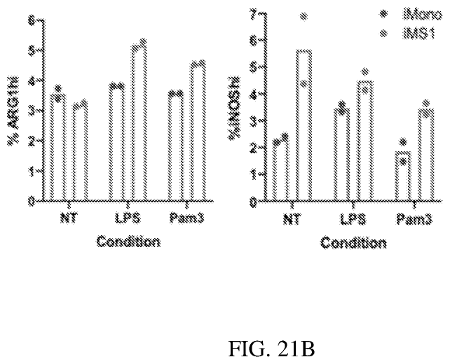

To compare the phenotype of MS1 cells with the myeloid-derived suppressor cells (M-MDSCs) known in the art, the levels of reactive oxygen species (ROS) were first detected in both MS1 cell and iMono cells by conducting MitoSOX-based assays with either MitoSOX-Red or Mito Tracker Green. As shown in FIG. 21A, MS1 cells comprised higher levels of ROS compared with iMono cells. The MS1 cells were then treated with the following groups in the culture media: (1) no treatment (NT), (2) LPS, or (3) Pam 3. As shown in FIG. 21B, MS1 resulted in higher % ARG1^{hi} and % iNOS^{hi} with the presence of LPS or Pam3. Interestingly, MS1 cells resulted in substantially higher % iNOS^{hi} even without any treatment. Taken together, the results showed that the MS1 phenotypes were consistent with the phenotypes of M-MDSCs with high ARG1, iNOs, and ROS.

### REFERENCES

1. Rudd, K. E. et al. The global burden of sepsis: barriers and potential solutions. Crit. Care 22, 232 (2018).
2. Singer, M. et al. The Third International Consensus Definitions for Sepsis and Septic Shock (Sepsis-3). JAMA 315, 801-810 (2016).
3. Angus, D. C. & van der Poll, T. Severe sepsis and septic shock. N. Engl. J. Med. 369, 840-851 (2013).
4. Filbin, M. R. et al. Presenting Symptoms Independently Predict Mortality in Septic Shock: Importance of a Previously Unmeasured Confounder. Crit. Care Med. 46, 1592-1599 (2018).
5. Seymour, C. W. et al. Derivation, Validation, and Potential Treatment Implications of Novel Clinical Phenotypes for Sepsis. JAMA (2019). doi:10.1001/jama.2019.5791
6. Sweeney, T. E. et al. Unsupervised Analysis of Transcriptomics in Bacterial Sepsis Across Multiple Datasets Reveals Three Robust Clusters. Crit. Care Med. 46, 915-925 (2018).
7. Opal, S. M., Dellinger, R. P., Vincent, J.-L., Masur, H. & Angus, D. C. The next generation of sepsis clinical trial designs: what is next after the demise of recombinant human activated protein C?*. Crit. Care Med. 42, 1714-1721 (2014).
8. Scicluna, B. P. & Baillie, J. K. The Search for Efficacious New Therapies in Sepsis Needs to Embrace Heterogeneity. Am. J. Respir. Crit. Care Med. (2018). doi:10.1164/rccm.201811-2148ED
9. Prescott, H. C., Calfee, C. S., Thompson, B. T., Angus, D. C. & Liu, V. X. Toward Smarter Lumping and Smarter Splitting: Rethinking Strategies for Sepsis and Acute Respiratory Distress Syndrome Clinical Trial Design. Am. J. Respir. Crit. Care Med. 194, 147-155 (2016).
10. Coopersmith, C. M. et al. Surviving sepsis campaign: research priorities for sepsis and septic shock. Intensive Care Med. (2018). doi:10.1007/s00134-018-5175-z
11. Davenport, E. E. et al. Genomic landscape of the individual host response and outcomes in sepsis: a prospective cohort study. Lancet Respir Med 4, 259-271 (2016).
12. Sweeney, T. E. & Khatri, P. Benchmarking Sepsis Gene Expression Diagnostics Using Public Data. Crit. Care Med. 45, 1-10 (2017).
13. Raymond, S. L. et al. Unique transcriptomic response to sepsis is observed among patients of different age groups. PLoS One 12, e0184159 (2017).
14. McHugh, L. et al. A Molecular Host Response Assay to Discriminate Between Sepsis and Infection-Negative Systemic Inflammation in Critically Ill Patients: Discovery and Validation in Independent Cohorts. PLoS Med. 12, e1001916 (2015).
15. Sweeney, T. E. et al. A community approach to mortality prediction in sepsis via gene expression analysis. Nat. Commun. 9, 694 (2018).
16. Reyes, M. et al. Multiplexed enrichment and genomic profiling of peripheral blood cells reveal subset-specific immune signatures. Science Advances 5, eaau9223 (2019).
17. Becker, A. M. et al. SLE peripheral blood B cell, T cell and myeloid cell transcriptomes display unique profiles and each subset contributes to the interferon signature. PLoS One 8, e67003 (2013).
18. Flint, S. M. et al. Leucocyte subset-specific type 1 interferon signatures in SLE and other immune-mediated diseases. RMD Open 2, e000183 (2016).
19. Center for Medicare & Medicaid Services (CMS). Sepsis Bundle Project (SEP), National Hospital Inpatient Quality Measures. Quality Net Available at: http://www.qualitynet.org/. (Accessed: 18 Jun. 2019)
20. Levy, M. M. et al. 2001 SCCM/ESICM/ACCP/ATS/SIS International Sepsis Definitions Conference. Intensive Care Med. 29, 530-538 (2003).
21. Stoeckius, M. et al. Cell Hashing with barcoded antibodies enables multiplexing and doublet detection for single cell genomics. Genome Biol. 19, 224 (2018).
22. Villani, A.-C. et al. Single-cell RNA-seq reveals new types of human blood dendritic cells, monocytes, and progenitors. Science 356, eaah4573-eaah4573 (2017).
23. Crinier, A. et al. High-Dimensional Single-Cell Analysis Identifies Organ-Specific Signatures and Conserved NK Cell Subsets in Humans and Mice. Immunity 49, 971-986.e5 (2018).
24. Zheng, G. X. Y. et al. Massively parallel digital transcriptional profiling of single cells. Nat. Commun. 8, 14049 (2017).
25. Sundén-Cullberg, J. et al. Pronounced elevation of resistin correlates with severity of disease in severe sepsis and septic shock. Crit. Care Med. 35, 1536-1542 (2007).
26. Macdonald, S. P. J. et al. Resistin and NGAL are associated with inflammatory response, endothelial activation and clinical outcomes in sepsis. Inflamm. Res. 66, 611-619 (2017).
27. Khattab, A. A., El-Mekkawy, M. S., Helwa, M. A. & Omar, E. S. Utility of serum resistin in the diagnosis of neonatal sepsis and prediction of disease severity in term and late preterm infants. J. Perinat. Med. 46, 919-925 (2018).
28. Lang, Y. et al. Interleukin-1 Receptor 2: A New Biomarker for Sepsis Diagnosis and Gram-Negative/Gram-Positive Bacterial Differentiation. Shock 47, 119-124 (2017).
29. Schaack, D., Siegler, B. H., Tamulyte, S., Weigand, M. A. & Uhle, F. The immunosuppressive face of sepsis early on intensive care unit-A large-scale microarray meta-analysis. PLoS One 13, e0198555 (2018).
30. Bauer, M. et al. A Transcriptomic Biomarker to Quantify Systemic Inflammation in Sepsis—A Prospective Multicenter Phase II Diagnostic Study. EBioMedicine 6, 114-125 (2016).
31. Sweeney, T. E., Shidham, A., Wong, H. R. & Khatri, P. A comprehensive time-course-based multicohort analysis of sepsis and sterile inflammation reveals a robust diagnostic gene set. Sci. Transl. Med. 7, 287ra71 (2015).
32. Kotliar, D. et al. Identifying gene expression programs of cell-type identity and cellular activity with single-cell RNA-Seq. Elife 8, 310599 (2019).
33. Ulas, T. et al. S100-alarmin-induced innate immune programming protects newborn infants from sepsis. Nat. Immunol. 18, 622-632 (2017).
34. Jang, J. C. et al. Human resistin protects against endotoxic shock by blocking LPS-TLR4 interaction. Proc. Natl. Acad. Sci. U.S.A. 114, E10399-E10408 (2017).
35. Orr, S. K. et al. Gene Expression of Proresolving Lipid Mediator Pathways Is Associated With Clinical Outcomes in Trauma Patients. Crit. Care Med. 43, 2642-2650 (2015).
36. Hotchkiss, R. S., Monneret, G. & Payen, D. Sepsis-induced immunosuppression: from cellular dysfunctions to immunotherapy. Nat. Rev. Immunol. 13, 862-874 (2013).
37. Hotchkiss, R. S., Monneret, G. & Payen, D. Immunosuppression in sepsis: a novel understanding of the disorder and a new therapeutic approach. Lancet Infect. Dis. 13, 260-268 (2013).
38. Scicluna, B. P. et al. A molecular biomarker to diagnose community-acquired pneumonia on intensive care unit admission. Am. J. Respir. Crit. Care Med. 192, 826-835 (2015).
39. Newman, A. M. et al. Robust enumeration of cell subsets from tissue expression profiles. Nat. Methods 12, 453-457 (2015).
40. Roy Chowdhury, R. et al. A multi-cohort study of the immune factors associated with M. tuberculosis infection outcomes. Nature 560, 644-648 (2018).
41. Uhel, F. et al. Early Expansion of Circulating Granulocytic Myeloid-derived Suppressor Cells Predicts Development of Nosocomial Infections in Patients with Sepsis. Am. J. Respir. Crit. Care Med. 196, 315-327 (2017).
42. Gossez, M. et al. Proof of concept study of mass cytometry in septic shock patients reveals novel immune alterations. Sci. Rep. 8, 17296 (2018).
43. Landelle, C. et al. Low monocyte human leukocyte antigen-DR is independently associated with nosocomial infections after septic shock. Intensive Care Med. 36, 1859-1866 (2010).
44. Veglia, F., Perego, M. & Gabrilovich, D. Myeloid-derived suppressor cells coming of age. Nat. Immunol. 19, 108-119 (2018).
45. Schrijver, I. T., Théroude, C. & Roger, T. Myeloid-Derived Suppressor Cells in Sepsis. Front. Immunol. 10, 327 (2019).
46. Chavakis, T., Mitroulis, I. & Hajishengallis, G. Hematopoietic progenitor cells as integrative hubs for adaptation to and fine-tuning of inflammation. Nat. Immunol. 20, 802-811 (2019).
47. Zhao, J. L. et al. Conversion of danger signals into cytokine signals by hematopoietic stem and progenitor cells for regulation of stress-induced hematopoiesis. Cell Stem Cell 14, 445-459 (2014).
48. Pastille, E. et al. Modulation of dendritic cell differentiation in the bone marrow mediates sustained immunosuppression after polymicrobial sepsis. J. Immunol. 186, 977-986 (2011).
49. Rodriguez, S. et al. Dysfunctional expansion of hematopoietic stem cells and block of myeloid differentiation in lethal sepsis. Blood 114, 4064-4076 (2009).
50. Zhang, H. et al. Sepsis Induces Hematopoietic Stem Cell Exhaustion and Myelosuppression through Distinct Contributions of TRIF and MYD88. Stem Cell Reports 6, 940-956 (2016).
51. McPeak, M. B. et al. Frontline Science: Myeloid cell-specific deletion of Cebpb decreases sepsis-induced immunosuppression in mice. J. Leukoc. Biol. 102, 191-200 (2017).
52. Rosenbauer, F. & Tenen, D. G. Transcription factors in myeloid development: balancing differentiation with transformation. Nat. Rev. Immunol. 7, 105-117 (2007).
53. Mitroulis, I., Kalafati, L., Hajishengallis, G. & Chavakis, T. Myelopoiesis in the Context of Innate Immunity. J. Innate Immun. 10, 365-372 (2018).
54. Manz, M. G. & Boettcher, S. Emergency granulopoiesis. Nat. Rev. Immunol. 14, 302-314 (2014).
55. Mathias, B. et al. Human Myeloid-derived Suppressor Cells are Associated With Chronic Immune Suppression After Severe Sepsis/Septic Shock. Ann. Surg. 265, 827-834 (2017).
56. Janols, H. et al. A high frequency of MDSCs in sepsis patients, with the granulocytic subtype dominating in gram-positive cases. J. Leukoc. Biol. 96, 685-693 (2014).
57. Wolf, F. A., Angerer, P. & Theis, F. J. SCANPY: large-scale single-cell gene expression data analysis. Genome Biol. 19, 15 (2018).
58. Vallania, F. et al. Leveraging heterogeneity across multiple datasets increases cell-mixture deconvolution accuracy and reduces biological and technical biases. Nat. Commun. 9, 4735 (2018).
59. Haynes, W. A. et al. Empowering Multi-Cohort Gene Expression Analysis to Increase Reproducibility. in Biocomputing 2017 144-153 (World Scientific, 2016).
60. Corces, M. R. et al. An improved ATAC-seq protocol reduces background and enables interrogation of frozen tissues. Nat. Methods 14, 959-962 (2017).
61. Picelli, S. et al. Full-length RNA-seq from single cells using Smart-seq2. Nat. Protoc. 9, 171-181 (2014).
62. Nakahira, K. et al. Circulating mitochondrial DNA in patients in the ICU as a marker of mortality: derivation and validation. PLoS Med. 10, e1001577; discussion e1001577 (2013).
63. Dolinay, T. et al. Inflammasome-regulated cytokines are critical mediators of acute lung injury. Am. J. Respir. Crit. Care Med. 185, 1225-1234 (2012).
64. Shalova, I. N. et al. Human monocytes undergo functional re-programming during sepsis mediated by hypoxia-inducible factor-1α. Immunity 42, 484-498 (2015).
65. André, M. C. et al. Bacterial reprogramming of PBMCs impairs monocyte phagocytosis and modulates adaptive T cell responses. J. Leukoc. Biol. 91, 977-989 (2012).
66. Ferreira da Mota, N. V. et al. Immunophenotyping of Monocytes During Human Sepsis Shows Impairment in Antigen Presentation: A Shift Toward Nonclassical Differentiation and Upregulation of FCγRi-Receptor. Shock 50, 293-300 (2018).
67. Santos, S. S. et al. Modulation of monocytes in septic patients: preserved phagocytic activity, increased ROS and NO generation, and decreased production of inflammatory cytokines. Intensive Care Med Exp 4, 5 (2016).
68. Brudecki, L., Ferguson, D. A., McCall, C. E. & El Gazzar, M. Myeloid-derived suppressor cells evolve during sepsis and can enhance or attenuate the systemic inflammatory response. Infect. Immun. 80, 2026-2034 (2012).
69. Van Wyngene, L., Vandewalle, J. & Libert, C. Reprogramming of basic metabolic pathways in microbial sepsis: therapeutic targets at last? EMBO Mol. Med. 10, (2018).
70. Khan, M. M., Yang, W.-L. & Wang, P. Endoplasmic reticulum stress in sepsis. Shock 44, 294-304 (2015).
71. Rosen, D. A. et al. Modulation of the sigma-1 receptor-IRE1 pathway is beneficial in preclinical models of inflammation and sepsis. Sci. Transl. Med. 11, (2019).
72. Ramilowski, J. A. et al. A draft network of ligand-receptor-mediated multicellular signalling in human. Nat. Commun. 6, 7866 (2015).

TABLE 1
Clinical characteristics of patient cohorts

| | Cohort: | | | | | | | |
|---|---|---|---|---|---|---|---|---|
| | Control | | Leuk-UTI | | Int-URO | | URO | |
| Patient count: | N = 19 | | N = 10 | | N = 7 | | N = 10 | |
| Demographics: | | | | | | | | |
| Age, Median Year | 57 | [51-66] | 53 | [33-66] | 71 | [54-75] | 69 | [55-75] |
| [interquartile range (IQR)] | | | | | | | | |
| Male, n (%) | 7 | (37%) | 4 | (40%) | 1 | (14%) | 6 | (60%) |
| White | 19 | (100%) | 10 | (100%) | 7 | (100%) | 10 | (100%) |

| Underlying medical | | | | | | | |
|---|---|---|---|---|---|---|---|
| comorbidities: | | | | | | | |
| Active cancer* | n.d.§ | 1 | (10%) | 1 | (14%) | 1 | (10%) |
| Immunocompromising | n.d. | 2 | (20%) | 1 | (14%) | 1 | (10%) |
| conditions** | | | | | | | |
| Coronary artery disease or | n.d. | 1 | (10%) | 3 | (43%) | 5 | (50%) |
| congestive heart failure | | | | | | | |
| Chronic kidney disease*** | n.d. | 0 | (0%) | 2 | (29%) | 1 | (10%) |
| Diabetes mellitus | n.d. | 1 | (10%) | 1 | (14%) | 3 | (30%) |
| Chronic severe lung | n.d. | 0 | (0%) | 1 | (14%) | 1 | (10%) |
| disease | | | | | | | |
| Clinical information on | | | | | | | |
| day of enrollment: | | | | | | | |
| Hours [IQR] from hospital | — | 3.9 | [3.1-4.9] | 4.0 | [3.6-7.1] | 4.3 | [2.9-5.1] |
| arrival to enrollment | | | | | | | |
| Hours from initial | — | 0.5 | [−1.6-1.0] | 1.7 | [1.2-3.7] | 1.8 | [−0.2-3.4] |
| antibiotic initiation | | | | | | | |
| Documented T >=100.4 F. | — | 4 | (40%) | 4 | (57%) | 3 | (30%) |
| Documented SBP <90 mmHg | — | 1 | (10%) | 3 | (43%) | 8 | (80%) |
| SOFA Score ****, Median | — | 0 | [0-1] | 2 | [2-3] | 4 | [2-6] |
| [IQR] | | | | | | | |
| WBC Count, Median | — | 14.6 | [13.9-14.8] | 12.0 | [8.9-17.0] | 15.6 | [14.0-19.5] |
| [IQR] | | | | | | | |
| % Lymphocytes [IQR] | — | 10% | [6-14] | 6% | [5-12] | 5% | [3-11] |
| % Monocytes [IQR] | — | 8% | [7-11] | 7% | [6-8] | 5% | [4-7] |
| Elevated serum | — | 0 | (0%) | 3 | (43%) | 6 | (60%) |
| lactate >2.0 mmol/dL | | | | | | | |
| Vasopressor therapy within | — | 0 | (0%) | 0 | (0%) | 3 | (30%) |
| 48 hours | | | | | | | |
| Identified infectious | | | | | | | |
| source: | | | | | | | |
| Pulmonary/pneumonia | — | 0 | (0%) | 0 | (0%) | 0 | (0%) |
| Abdominal | — | 0 | (0%) | 0 | (0%) | 0 | (0%) |
| Urinary tract | — | 10 | (100%) | 7 | (100%) | 10 | (100%) |
| Wound/Soft Tissue/Skin | — | 0 | (0%) | 0 | (0%) | 0 | (0%) |
| Endocarditis | — | 0 | (0%) | 0 | (0%) | 0 | (0%) |
| Unclear source | — | 0 | (0%) | 0 | (0%) | 0 | (0%) |
| Microorganism | | | | | | | |
| Gram-negative | — | 8 | (80%) | 4 | (57%) | 9 | (90%) |
| Escherichia coli | — | 5 | (50%) | 3 | (43%) | 6 | (60%) |
| Klebsiella | — | 2 | (20%) | 1 | (14%) | — | |
| pneumoniae | | | | | | | |
| Citrobacter koseri | — | 1 | (10%) | — | | — | |
| Enterobacter cloacae | — | — | | — | | 2 | (20%) |
| complex | | | | | | | |
| Bacteroides fragilis | — | — | | — | | — | |
| Proteus mirabilis | — | — | | — | | 1 | (10%) |
| Enterobacter | | | | | | | |
| aerogenes | | | | | | | |
| Gram-positive | — | 1 | (10%) | 1 | (14%) | 1 | (10%) |
| Staphylococcus | — | — | | 1 | (14%) | — | |
| Enterococcus | — | 1 | (10%) | — | | 1 | (10%) |
| Streptococcus | — | — | | — | | — | |
| No pathogen isolated | — | 1 | (1%) | 2 | (28%) | 0 | (0%) |
| Clinical outcomes | | | | | | | |
| variables: | | | | | | | |
| Admission to an ICU | — | 0 | (0%) | 0 | (0%) | 3 | (30%) |
| within 48 hrs | | | | | | | |
| Positive Blood Culture | — | 0 | (0%) | 2 | (29%) | 5 | (50%) |
| Death during index illness | — | 0 | (0%) | 0 | (0%) | 1 | (10%) |
| and/or hospitalization | | | | | | | |

| | Cohort: | | | | | |
|---|---|---|---|---|---|---|
| | Bac-SEP | | ICU-SEP | | ICU-NoSEP | |
| Patient count: | N = 4 | | N = 8 | | N = 7 | |
| Demographics: | | | | | | |
| Age, Median Year | 64 | [58-73] | 63 | [59-68] | 60 | [43-66] |
| [interquartile range (IQR)] | | | | | | |
| Male, n (%) | 4 | (100%) | 6 | (75%) | 4 | (57%) |
| White | 3 | (75%) | 4 | (50%) | 3 | (43%) |
| Underlying medical | | | | | | |
| comorbidities: | | | | | | |
| Active cancer* | 1 | (25%) | 3 | (38%) | 1 | (14%) |
| Immunocompromising | 0 | (0%) | 2 | (25%) | 1 | (14%) |
| conditions** | | | | | | |
| Coronary artery disease or | 1 | (25%) | 3 | (38%) | 3 | (43%) |
| congestive heart failure | | | | | | |
| Chronic kidney disease*** | 0 | (0%) | 2 | (25%) | 2 | (29%) |
| Diabetes mellitus | 0 | (0%) | 5 | (63%) | 3 | (43%) |
| Chronic severe lung | 0 | (0%) | 1 | (13%) | 3 | (43%) |
| disease | | | | | | |
| Clinical information on | | | | | | |
| day of enrollment: | | | | | | |
| Hours [IQR] from hospital | 70 | [57-83] | 68 | [44-100] | 41 | [34-58] |
| arrival to enrollment | | | | | | |
| Hours from initial | 70 | [61-79] | 49 | [44-65] | 49 | [35-135]§§ |
| antibiotic initiation | | | | | | |
| Documented T >=100.4 F. | 0 | (0%) | 3 | (38%) | 0 | (0%) |
| Documented SBP <90 mmHg | 0 | (0%) | 2 | (25%) | 1 | (14%) |
| SOFA Score ****, Median | 2 | [0-3] | 4 | [3-4] | 1 | [1-4] |
| [IQR] | | | | | | |
| WBC Count, Median | 10.3 | [9.5-13.4] | 10.0 | [7.9-19.9] | 8.5 | [7.9-16.3] |
| [IQR] | | | | | | |
| % Lymphocytes [IQR] | 10% | (6-11] | 7% | [5-9] | 20% | [19-31] |
| % Monocytes [IQR] | 9% | [8-10] | 5% | [2-8] | 8% | [7-10] |
| Elevated serum | 0 | (0%) | 0 | (0%) | 0 | (0%) |
| lactate >2.0 mmol/dL | | | | | | |
| Vasopressor therapy within | 0 | (0%) | 1 | (13%) | 0 | (0%) |
| 48 hours | | | | | | |
| Identified infectious | | | | | | |
| source: | | | | | | |
| Pulmonary/pneumonia | 0 | (0%) | 3 | (38%) | 0 | (0%) |
| Abdominal | 2 | (50%) | 1 | (13%) | 0 | (0%) |
| Urinary tract | 0 | (0%) | 1 | (13%) | 0 | (0%) |
| Wound/Soft Tissue/Skin | 0 | (0%) | 0 | (0%) | 0 | (0%) |
| Endocarditis | 2 | (50%) | 0 | (0%) | 0 | (0%) |
| Unclear source | 0 | (0%) | 2 | (25%) | 0 | (0%) |
| Microorganism | | | | | | |
| Gram-negative | 0 | (0%) | 3 | (38%) | — | |
| Escherichia coli | — | | 1 | (12%) | — | |
| Klebsiella | — | | — | | — | |
| pneumoniae | | | | | | |
| Citrobacter koseri | — | | — | | — | |
| Enterobacter cloacae | — | | — | | — | |
| complex | | | | | | |
| Bacteroides fragilis | — | | 1 | (12%) | — | |
| Proteus mirabilis | — | | — | | — | |
| Enterobacter | | | 1 | (12%) | | |
| aerogenes | | | | | | |
| Gram-positive | 4 | (100%) | 4 | (50%) | — | |
| Staphylococcus | 1 | (25%) | 1 | (12%) | — | |
| Enterococcus | 2 | (50%) | — | | — | |
| Streptococcus | 1 | (25%) | 3 | (38%) | — | |
| No pathogen isolated | 0 | (0%) | 1 | (12%) | — | |
| Clinical outcomes | | | | | | |
| variables: | | | | | | |
| Admission to an ICU | 0 | (0%) | 8 | (100%) | 7 | (100%) |
| within 48 hrs | | | | | | |
| Positive Blood Culture | 4 | (100%) | 5 | (63%) | 0 | (0%) |
| Death during index illness | 0 | (0%) | 1 | (12%) | 0 | (0%) |
| and/or hospitalization | | | | | | |

*Active treatment for cancer diagnosis or metastatic disease.
**Immunocompromising conditions include receipt of chemotherapy within 30 days, organ transplant, chronic condition requiring immunomodulating therapy, or splenectomy. Specifically, 2 Leuk-UTI patients had prior splenectomy, 1 Int-URO patient and 1 URO patient were on chemotherapy for active cancer, 1 ICU-SEP patient was on immunosuppressants for a renal transplant, and 1 ICU-NoSEP patient was on low-dose prednisone for polymyositis.
***Denotes at least stage 3 chronic kidney disease with glomerular filtration rate <60 mL/min.
**** Sequential organ failure assessment (SOFA) score is a standard for grading illness severity and is based on functional status of 6 organ systems. [Vincent, J. L. et al. Intensive Care Med. 22, 707-710 (1996)]
§Not done: Matched controls were volunteers who donated blood while they were not ill, and no further information is available on their underlying medical comorbidities.
§§Four of 7 ICU-NoSEP patients had antibiotics started during hospitalization but were later determined not to be infected.

TABLE 2
MS1 Marker Genes

| | | |
|---|---|---|
| Gene | logFC | FDR |

MS1 vs. All Monocytes

| | | |
|---|---|---|
| RETN | 2.6071298 | 0 |
| ALOX5AP | 1.8590558 | 0 |
| CD63 | 1.030687 | 0 |
| SEC61G | 0.771678 | 3.15E−202 |
| TXN | 0.7650104 | 1.76E−149 |
| MT1X | 1.2253797 | 7.59E−134 |
| FOS | 0.71384716 | 2.07E−127 |
| SOD2 | 0.7368248 | 4.64E−112 |
| NCF1 | 0.6139928 | 4.11E−97 |
| IL1R2 | 3.1905918 | 1.76E−71 |
| THBS1 | 1.9180616 | 6.79E−69 |
| DPYSL2 | 0.624073 | 4.39E−61 |
| PTPRE | 0.5733434 | 8.21E−61 |
| C6orf62 | 0.5696041 | 2.36E−51 |
| FES | 0.9147935 | 3.14E−47 |
| CD164 | 0.48924762 | 4.01E−43 |
| TM9SF2 | 0.597988 | 1.32E−38 |
| PRKAR1A | 0.47393256 | 7.06E−37 |
| SLC25A37 | 0.5977127 | 3.23E−35 |
| YWHAE | 0.3587922 | 2.00E−31 |
| PTEN | 0.4516738 | 1.04E−30 |
| SLC38A2 | 0.6691931 | 1.54E−30 |
| A1BG | 0.5683889 | 1.68E−27 |
| CSNK1A1 | 0.4231965 | 1.18E−26 |
| ADAM10 | 0.4963114 | 3.94E−26 |
| TRABD | 0.6142366 | 1.90E−25 |
| LILRA5 | 0.28308862 | 2.01E−25 |
| TLN1 | 0.34133977 | 6.72E−25 |
| DYNC1I2 | 0.58676225 | 1.43E−23 |
| CSF2RB | 0.73332447 | 4.70E−23 |
| ITGAE | 0.5312276 | 2.36E−22 |
| IL1RN | 1.0305688 | 3.47E−20 |
| LPXN | 0.63793373 | 4.30E−19 |
| RRBP1 | 0.7597774 | 4.80E−19 |
| SNHG25 | 0.40689662 | 5.58E−19 |
| FOSL2 | 0.46375927 | 5.71E−19 |
| CAPZA1 | 0.25094187 | 2.17E−18 |
| FKBP2 | 0.35492882 | 3.95E−18 |
| HNRNPM | 0.4046929 | 2.19E−17 |
| GOLPH3 | 0.58164346 | 3.86E−17 |
| JAK3 | 0.66083044 | 7.41E−16 |
| SLC16A7 | 1.011987 | 9.43E−16 |
| ESRRA | 0.23770268 | 1.16E−12 |
| YY1 | 0.3434706 | 8.85E−12 |
| RAB1B | 0.50652397 | 4.16E−11 |
| MYL12A | 0.34635365 | 5.50E−11 |
| NUFIP2 | 0.5506808 | 5.50E−11 |
| CTBP1 | 0.38685283 | 7.55E−10 |
| BIRC2 | 0.352858 | 8.64E−10 |
| SLC15A4 | 0.89066195 | 1.32E−09 |
| LENG8 | 0.2823143 | 1.33E−09 |
| TDG | 0.61318463 | 1.46E−09 |
| DNAJC3 | 0.3965807 | 3.00E−09 |
| COLGALT1 | 0.52488637 | 3.71E−09 |
| TNFAIP6 | 2.4990368 | 5.73E−09 |
| TMEM33 | 0.39364615 | 7.59E−09 |
| APP | 0.6245647 | 7.72E−09 |
| UQCC3 | 0.3710911 | 9.73E−09 |
| OGT | 0.4327405 | 4.28E−08 |
| DNAJC7 | 0.2860722 | 4.59E−08 |
| CST7 | 0.38517067 | 5.20E−08 |
| EIF3J | 0.34985632 | 6.48E−08 |
| CERS6 | 0.75459033 | 9.84E−08 |
| TBL1XR1 | 0.36999843 | 3.82E−07 |
| IRAK1 | 0.6889536 | 4.51E−07 |
| YTHDF3 | 0.65564895 | 5.89E−07 |
| ADAM17 | 0.34209844 | 6.45E−07 |
| ST14 | 1.0434066 | 1.21E−06 |
| SMG7 | 0.6179948 | 1.24E−06 |
| PUM2 | 0.47554237 | 1.42E−06 |
| PRPF4B | 0.29350668 | 2.07E−06 |
| CDK2AP2 | 0.23574242 | 2.39E−06 |
| PHACTR2 | 0.59475875 | 4.78E−06 |
| MGST2 | 0.3368151 | 5.59E−06 |
| KIAA0319L | 0.617746 | 7.38E−06 |
| ABCA7 | 0.6656912 | 7.58E−06 |
| MGAT4A | 0.64594626 | 7.67E−06 |
| SFT2D2 | 0.29606098 | 7.67E−06 |
| COX20 | 0.30872056 | 8.14E−06 |
| TES | 0.25897893 | 1.12E−05 |
| MT1G | 3.050509 | 1.67E−05 |
| AP2B1 | 0.43252102 | 1.79E−05 |
| ADAM19 | 0.86898 | 1.88E−05 |
| HIP1 | 1.147587 | 1.88E−05 |
| PABPN1 | 0.19034192 | 2.12E−05 |
| NCOA3 | 0.42164952 | 2.75E−05 |
| INTS6 | 0.6486802 | 5.27E−05 |
| UPF2 | 0.23221949 | 6.82E−05 |
| METAP2 | 0.23328802 | 7.14E−05 |
| BRI3BP | 0.5740479 | 9.89E−05 |
| NUP50 | 0.35948458 | 0.000115735 |
| HIST1H4C | 0.3765645 | 0.000126003 |
| IGHA1 | 0.79512155 | 0.000136945 |
| HBB | 2.4754834 | 0.000136945 |
| APMAP | 0.6145643 | 0.000138174 |
| EXOC5 | 0.70440876 | 0.000142635 |
| CHD3 | 0.59686637 | 0.000160344 |
| PNKD | 0.19284436 | 0.000168263 |
| FBXW5 | 0.2568183 | 0.000170603 |
| MAPKAPK2 | 0.63853717 | 0.000174336 |
| PTPN22 | 0.9228026 | 0.000174336 |
| TNKS2 | 0.41274077 | 0.00020333 |
| ARF4 | 0.32980248 | 0.000221394 |
| ATP2A3 | 0.73342407 | 0.000255298 |
| POR | 0.65733504 | 0.000292881 |
| MT-ATP8 | 0.30359328 | 0.000299804 |
| ADAM8 | 0.6757972 | 0.000313421 |
| MKL1 | 0.5750798 | 0.000345181 |
| SUPT5H | 0.4637095 | 0.000406766 |
| FBXL15 | 0.20853965 | 0.000468876 |
| DNAJB11 | 0.15336898 | 0.00055291 |
| HNRNPAB | 0.33968168 | 0.000601991 |
| TRAM1 | 0.15445973 | 0.000610102 |
| DNM2 | 0.36547217 | 0.000647249 |
| STAG2 | 0.14570394 | 0.000770388 |
| HERPUD1 | 0.2179522 | 0.000892666 |
| RSBN1L | 0.3034676 | 0.001032879 |
| DR1 | 0.27376887 | 0.001243421 |
| MAN2A1 | 0.45276383 | 0.00135465 |
| YTHDC1 | 0.2812023 | 0.001407315 |
| GSN | 0.49243438 | 0.001542903 |
| NUCB2 | 0.6305114 | 0.001666061 |
| LLNLR-245B6.1 | 0.292182 | 0.001723708 |
| COPG1 | 0.5355667 | 0.001803713 |
| PTPRA | 0.37326708 | 0.001830175 |
| AP2A2 | 0.54962873 | 0.002277773 |
| CBX6 | 0.18427254 | 0.003037842 |
| AC004556.1 | 0.3473703 | 0.003077118 |
| RREB1 | 0.3386614 | 0.003297111 |
| NXF1 | 0.3765875 | 0.003550557 |
| UHMK1 | 0.279729 | 0.003646805 |
| TRG-AS1 | 0.5669947 | 0.003841836 |
| DENND4B | 0.3269256 | 0.00392757 |
| AP3M1 | 0.48430827 | 0.003979046 |
| COMTD1 | 0.48372906 | 0.004392478 |
| PLCB2 | 0.2383028 | 0.004673064 |
| CNOT1 | 0.34327912 | 0.005009646 |
| HYOU1 | 0.99043995 | 0.005108712 |
| RHBDF2 | 0.22131197 | 0.005609798 |
| PTMS | 0.64900255 | 0.005667637 |
| PRKAA1 | 0.4495149 | 0.005853431 |
| TYK2 | 0.33223215 | 0.006018868 |
| KIAA0100 | 0.4348752 | 0.007495297 |
| DCTN1 | 0.5351371 | 0.0082376 |
| XPO1 | 0.27625823 | 0.008719539 |
| FBXW11 | 0.44099098 | 0.009324827 |
| CYTH1 | 0.23365675 | 0.009804773 |
| SMG1 | 0.22367293 | 0.010071709 |
| UBE4A | 0.41928113 | 0.010071709 |
| HOPX | 0.45225978 | 0.010255791 |
| ARL6IP1 | 0.2114991 | 0.010381054 |
| HSPB1 | 0.11302291 | 0.011133753 |
| NLRC5 | 0.37520042 | 0.011724672 |
| IRF2BPL | 0.15934825 | 0.012917572 |
| SPTY2D1 | 0.6268313 | 0.012920543 |
| ANP32E | 0.31182006 | 0.014645808 |
| AVL9 | 0.49529022 | 0.015434448 |
| CH17-373J23.1 | 0.29601997 | 0.018129658 |
| DDIT4 | 0.4636246 | 0.01849667 |
| ZMPSTE24 | 0.48480412 | 0.019816406 |
| PBX2 | 0.31123573 | 0.021372953 |
| ACADVL | 0.15724014 | 0.022213942 |
| POGZ | 0.48029622 | 0.023198724 |
| MED15 | 0.41280824 | 0.023198724 |
| AP1G1 | 0.2602707 | 0.023422158 |
| KRCC1 | 0.26675805 | 0.026988406 |
| TIAL1 | 0.24003243 | 0.032932773 |
| SEC23A | 0.3994531 | 0.034199997 |
| PSD4 | 0.41415885 | 0.035089338 |
| ZNF516 | 0.3728206 | 0.040525388 |
| MAP2K4 | 0.6783416 | 0.040525388 |
| RASSF3 | 0.015267988 | 0.042905897 |
| SMARCA5 | 0.26114753 | 0.04495159 |
| TTC1 | 0.2784163 | 0.047193603 |
| ITGB7 | 0.57286036 | 0.049123849 |
| AKT2 | 0.4165231 | 0.050533959 |
| DDA1 | 0.26530516 | 0.052896613 |
| PAFAH1B2 | 0.19234869 | 0.053169794 |
| HBA2 | 1.8376906 | 0.053260459 |
| ATF7IP | 0.288255 | 0.059343386 |
| VIMP | 0.1517431 | 0.063059392 |
| MIER1 | 0.20790116 | 0.063751163 |
| PRPF8 | 0.29201698 | 0.064128662 |
| MKLN1 | 0.35631666 | 0.065876041 |
| RPS6KA4 | 0.22100714 | 0.066109263 |
| SUDS3 | 0.36791486 | 0.067422484 |
| RSRC2 | 0.15604736 | 0.07709203 |
| MDM2 | 0.37148646 | 0.077484608 |
| STARD3NL | 0.19558172 | 0.078719463 |
| BORCS8 | 0.2594253 | 0.079383637 |
| SUGP2 | 0.3372654 | 0.081581496 |
| SUPT6H | 0.3959987 | 0.088522211 |
| MRPL55 | 0.095821485 | 0.091340501 |
| STT3A | 0.6315023 | 0.092602393 |
| TRIM22 | 0.12941799 | 0.092919266 |
| SUMF1 | 0.45330456 | 0.092958692 |
| DAB2 | 0.8046983 | 0.096667144 |
| TMX3 | 0.39931664 | 0.101796792 |
| SACM1L | 0.47407183 | 0.101941203 |
| PRDM2 | 0.28756317 | 0.109150211 |
| PPP3R1 | 0.118881494 | 0.113893872 |
| HGS | 0.32583207 | 0.119440539 |
| PREB | 0.447414 | 0.120584361 |
| PPP2R5E | 0.28165907 | 0.127432287 |
| RP11-802E16.3 | 0.3349683 | 0.127432287 |
| C2orf68 | 0.06591099 | 0.127581473 |
| MRPL53 | 0.08664803 | 0.129449182 |
| YIF1B | 0.16365983 | 0.132673079 |
| WBP4 | 0.3433745 | 0.133006981 |
| PIP4K2A | 0.12928657 | 0.133795107 |
| CRIPT | 0.225312 | 0.135705325 |
| CCDC186 | 0.39603543 | 0.1358341 |
| VPS26A | 0.32276332 | 0.137061291 |
| CDYL | 0.44439057 | 0.13725805 |
| MSL2 | 0.30856586 | 0.13725805 |
| HSPA1B | 0.5752229 | 0.142226563 |
| FUT7 | 0.79572237 | 0.142226563 |
| ZDHHC2 | 0.40883923 | 0.144967423 |
| ARHGAP9 | 0.13835016 | 0.146222542 |
| CYHR1 | 0.23022157 | 0.147959865 |
| CNOT4 | 0.30169037 | 0.151434525 |
| CSF2RA | 0.12082033 | 0.15147525 |
| PSMA3-AS1 | 0.085751 | 0.158552344 |
| TSPAN3 | 0.33060408 | 0.161297735 |
| FABP5 | 0.27277285 | 0.164975928 |
| ERLEC1 | 0.26383242 | 0.166953386 |
| GZMB | 0.10198447 | 0.174934599 |
| SNHG9 | 0.011241212 | 0.180074612 |
| LIMK2 | 0.87808233 | 0.185087586 |
| RC3H1 | 0.15169793 | 0.185087586 |
| RP11-140K17.3 | 0.25836122 | 0.187758748 |
| RNF220 | 0.23034744 | 0.198066198 |

MS1 vs. MS2

| | | |
|---|---|---|
| S100A8 | 1.2660923 | 0 |
| S100A12 | 1.7208534 | 0 |
| LGALS1 | 0.865564 | 0 |
| S100A9 | 1.022807 | 0 |
| CTSD | 1.4100318 | 0 |
| VCAN | 1.0673008 | 0 |
| RETN | 2.2511775 | 0 |
| S100A6 | 0.37841713 | 0 |
| MT-ND3 | 0.6360306 | 0 |
| ATP5E | 0.43313357 | 0 |
| STXBP2 | 1.0953611 | 0 |
| PLAC8 | 1.2963204 | 0 |
| MT-ATP6 | 0.56278205 | 0 |
| MT-CO2 | 0.34047136 | 0 |
| MT-ND4 | 0.4329782 | 0 |
| CLU | 2.801447 | 0 |
| MT-CO3 | 0.41353533 | 0 |
| SERF2 | 0.30509362 | 0 |
| CYP1B1 | 1.7745417 | 0 |
| SELL | 1.1894882 | 1.64E−278 |
| FTL | 0.2361522 | 2.22E−267 |
| MCEMP1 | 2.063787 | 1.87E−250 |
| C4orf48 | 0.6754157 | 8.76E−224 |
| MT-CO1 | 0.24811521 | 2.39E−217 |
| VIM | 0.45644048 | 1.93E−202 |
| NKG7 | 0.7985205 | 9.42E−187 |
| TMSB10 | 0.16941012 | 2.45E−173 |
| LCP1 | 0.58182216 | 3.94E−154 |
| RPL37A | 0.22607289 | 2.42E−153 |
| GAPDH | 0.30043817 | 6.33E−144 |
| RPL28 | 0.17074087 | 2.33E−138 |
| ATP5I | 0.44289914 | 5.75E−134 |
| LILRA5 | 1.0121979 | 1.61E−130 |
| NDUFB1 | 0.45673916 | 2.36E−128 |
| PLBD1 | 0.88771534 | 1.23E−127 |
| ACTG1 | 0.39311233 | 9.91E−126 |
| FAM65B | 0.8475277 | 5.62E−123 |
| RPLP2 | 0.15811726 | 1.61E−116 |
| ACTB | 0.16220982 | 2.25E−115 |
| MT-CYB | 0.3285237 | 2.53E−115 |
| RPLP1 | 0.14785194 | 1.03E−114 |
| ITGB2 | 0.42875078 | 1.11E−114 |
| ALOX5AP | 1.1115316 | 3.55E−109 |
| MT-ND1 | 0.2594698 | 1.23E−108 |
| C14orf2 | 0.24693733 | 1.06E−101 |
| CD36 | 0.5795429 | 3.24E−100 |
| PGD | 0.8032501 | 2.72E−97 |
| COX8A | 0.33336607 | 1.59E−96 |
| CTSB | 0.58019674 | 1.60E−92 |
| GCA | 0.5997053 | 1.03E−91 |
| RPL38 | 0.18344103 | 3.63E−90 |
| BLOC1S1 | 0.37446776 | 2.22E−84 |
| MT-ND5 | 0.34526667 | 4.17E−81 |
| PGAM1 | 0.604217 | 1.64E−79 |
| RPL39 | 0.10306641 | 3.49E−79 |
| ITGAM | 0.93438846 | 4.59E−79 |
| ACSL1 | 1.4605644 | 4.12E−76 |
| KCTD12 | 0.597197 | 4.83E−76 |
| TCEB2 | 0.20061368 | 7.75E−75 |
| FKBP5 | 1.1510907 | 3.68E−74 |
| CD163 | 1.1130005 | 8.58E−74 |
| RNASE2 | 0.95553434 | 1.34E−73 |
| AQP9 | 1.4926822 | 3.07E−71 |
| MT-ND2 | 0.23074804 | 4.32E−69 |
| CCND3 | 0.8582858 | 1.17E−68 |
| SLC39A8 | 3.2838552 | 1.98E−68 |
| POLR2L | 0.26666743 | 3.58E−68 |
| ROMO1 | 0.5869193 | 4.11E−68 |
| SERPINB1 | 0.47402006 | 2.60E−66 |
| RPL37 | 0.13436005 | 8.63E−66 |
| UQCR11 | 0.18127047 | 3.19E−65 |
| H2AFJ | 0.71873754 | 6.77E−64 |
| STAB1 | 1.1472969 | 8.66E−63 |
| SH3BGRL3 | 0.100744024 | 3.50E−60 |
| TMBIM6 | 0.4155807 | 4.37E−58 |
| APLP2 | 0.41087872 | 7.01E−56 |
| TPT1 | 0.08497499 | 6.25E−55 |
| CD63 | 0.32904074 | 2.41E−52 |
| PGK1 | 0.42703417 | 4.93E−52 |
| RPS21 | 0.132002 | 2.27E−50 |
| SLC11A1 | 0.6136691 | 3.26E−49 |
| ACTR2 | 0.35206395 | 6.28E−46 |
| GNS | 0.7832561 | 5.23E−45 |
| AGFG1 | 1.1471243 | 4.72E−44 |
| NDUFA3 | 0.31240466 | 1.75E−41 |
| TPM3 | 0.28905964 | 6.58E−41 |
| PKM | 0.3372666 | 2.05E−40 |
| ATP5EP2 | 0.46409822 | 8.39E−40 |
| LINC01272 | 0.4214121 | 1.08E−39 |
| CKAP4 | 1.1910115 | 4.11E−39 |
| CD55 | 0.60984516 | 1.12E−38 |
| ANXA6 | 0.9761884 | 3.09E−38 |
| MYH9 | 0.63186467 | 4.76E−38 |
| CYBB | 0.25911117 | 5.30E−38 |
| RAB31 | 0.5714583 | 7.24E−38 |
| IL1R2 | 2.8737311 | 4.20E−37 |
| COX17 | 0.4277356 | 3.59E−34 |
| LAPTM5 | 0.18337049 | 4.93E−34 |
| NUP214 | 0.28630164 | 8.51E−34 |
| GRINA | 0.6235833 | 1.43E−33 |
| EHBP1L1 | 0.74488795 | 1.06E−32 |
| THBS1 | 1.7519834 | 2.07E−32 |
| S100P | 1.867267 | 3.74E−32 |
| FGR | 0.3035864 | 7.75E−32 |
| NEAT1 | 0.11250022 | 5.61E−31 |
| USMG5 | 0.18516852 | 1.81E−30 |
| CAP1 | 0.32360518 | 2.44E−30 |
| TSPO | 0.1098452 | 5.26E−30 |
| MRPL52 | 0.37046593 | 2.61E−29 |
| SKAP2 | 0.5392888 | 1.62E−28 |
| WSB1 | 0.47894296 | 2.32E−28 |
| UBL5 | 0.13641965 | 2.82E−28 |
| CLTC | 0.62229115 | 6.38E−28 |
| MT1X | 0.7254027 | 1.12E−27 |
| S100A10 | 0.124558374 | 1.57E−27 |
| RP11-295G20.2 | 0.80856514 | 1.59E−27 |
| FAM198B | 0.68416363 | 3.53E−27 |
| HMGB2 | 0.3409171 | 4.29E−27 |
| PHC2 | 1.1575555 | 4.69E−27 |
| FPR2 | 1.0980574 | 5.03E−27 |
| CD44 | 0.22083831 | 5.88E−27 |
| ARPC4 | 0.31154835 | 8.86E−27 |
| TMPO | 0.82558805 | 1.44E−26 |
| ESRRA | 0.74989027 | 1.44E−26 |
| VNN2 | 0.7242236 | 4.04E−26 |
| ATOX1 | 0.33400074 | 4.63E−26 |
| UBA52 | 0.051680624 | 1.75E−24 |
| CD93 | 0.49930564 | 3.55E−24 |
| EFHD2 | 0.25738204 | 4.14E−24 |
| FLNA | 0.36406964 | 4.88E−24 |
| PIK3AP1 | 0.6617256 | 1.19E−23 |
| IFI27L2 | 0.34769565 | 1.87E−23 |
| SSH2 | 0.5974137 | 3.44E−23 |
| MSRB1 | 0.4238649 | 6.09E−23 |
| MS4A4A | 0.94841206 | 7.82E−23 |
| SEC61G | 0.16337833 | 1.51E−22 |
| NFE2 | 0.8735045 | 2.15E−22 |
| PIM1 | 0.90796894 | 3.09E−22 |
| MTRNR2L8 | 0.23801869 | 3.10E−22 |
| MEGF9 | 0.64096045 | 3.73E−22 |
| MT2A | 0.30687836 | 5.41E−22 |
| SLA | 0.8237741 | 7.35E−22 |
| MTRNR2L12 | 0.4806108 | 1.34E−21 |
| NAMPT | 0.490383 | 2.83E−21 |
| GNG5 | 0.16602293 | 5.49E−21 |
| YWHAZ | 0.23863004 | 9.98E−21 |
| SLC2A3 | 0.7472694 | 1.05E−20 |
| DAZAP2 | 0.1993865 | 1.24E−20 |
| TMEM167A | 0.26202032 | 1.78E−20 |
| RHOA | 0.104796804 | 2.60E−20 |
| MSN | 0.2812612 | 3.89E−20 |
| LILRB2 | 0.47168824 | 5.70E−20 |
| MT-ND4L | 0.3064619 | 5.77E−20 |
| RBM47 | 0.6339632 | 1.29E−19 |
| MTPN | 0.3277827 | 1.41E−19 |
| IRAK3 | 0.5486246 | 1.41E−19 |
| TMA7 | 0.053598724 | 1.45E−19 |
| PAG1 | 0.96878064 | 1.60E−19 |
| FPR1 | 0.31362 | 3.93E−19 |
| STOM | 1.0711293 | 5.35E−19 |
| CES1 | 0.8391904 | 5.90E−19 |
| HSPA5 | 0.6118494 | 5.99E−19 |
| MT1F | 1.1207933 | 7.39E−19 |
| LILRA6 | 1.0665289 | 7.41E−19 |
| CAPNS1 | 0.384402 | 1.20E−18 |
| SAMSN1 | 0.74217016 | 1.66E−18 |
| LASP1 | 0.64323115 | 3.15E−18 |
| CAPZA1 | 0.26949075 | 3.18E−18 |
| FAM101B | 0.94383883 | 4.02E−18 |
| TIMP2 | 0.4539843 | 4.17E−18 |
| SEPW1 | 0.40922415 | 4.29E−18 |
| EIF4G2 | 0.29823986 | 4.48E−18 |
| C20orf24 | 0.28632516 | 6.72E−18 |
| CD53 | 0.29650187 | 6.84E−18 |
| ZDHHC20 | 0.7075934 | 9.74E−18 |
| ANPEP | 1.0089974 | 1.76E−17 |
| SELPLG | 0.49166486 | 1.85E−17 |
| CMTM6 | 0.32754195 | 1.96E−17 |
| CALM3 | 0.25979975 | 3.71E−17 |
| ADM | 1.4483229 | 3.79E−17 |
| CTSA | 0.4654828 | 6.13E−17 |
| UPP1 | 0.48917496 | 6.45E−17 |
| LBR | 0.66398776 | 7.30E−17 |
| RPS12 | 0.053308938 | 7.79E−17 |
| CALR | 0.44803378 | 1.01E−16 |
| METTL9 | 0.2906079 | 1.02E−16 |
| LYN | 0.35207987 | 1.43E−16 |
| SOD2 | 0.33049357 | 1.59E−16 |
| PLEC | 0.6902505 | 2.23E−16 |
| RNF149 | 0.38784036 | 2.50E−16 |
| IL17RA | 0.45119062 | 4.01E−16 |
| B4GALT5 | 1.175831 | 4.43E−16 |
| MYEOV2 | 0.2025189 | 5.36E−16 |
| MXD1 | 0.7261478 | 1.19E−15 |
| DBI | 0.14654645 | 1.21E−15 |
| CHCHD7 | 0.6362854 | 1.49E−15 |
| HIPK2 | 0.9150807 | 3.97E−15 |
| MIR4435-2HG | 0.59805727 | 4.55E−15 |
| ACTR3 | 0.28953353 | 6.64E−15 |
| HP | 2.0137117 | 6.65E−15 |
| TPP1 | 0.32147458 | 2.89E−14 |
| IRS2 | 0.8072485 | 3.30E−14 |
| LILRB1 | 0.59630066 | 7.24E−14 |
| LDHA | 0.28115454 | 1.21E−13 |
| GNAI2 | 0.18774572 | 2.25E−13 |
| QSOX1 | 0.7426047 | 2.45E−13 |
| DYSF | 1.2174559 | 3.18E−13 |
| IQGAP1 | 0.23119423 | 6.53E−13 |
| LINC00657 | 0.4935494 | 7.77E−13 |
| MOB1A | 0.27084503 | 9.18E−13 |
| FLOT1 | 0.5359697 | 1.41E−12 |
| ZSCAN16-AS1 | 0.5322747 | 1.71E−12 |
| RP6-159A1.4 | 0.38520387 | 2.23E−12 |
| RASSF2 | 0.4552823 | 2.67E−12 |
| GNAQ | 0.45653936 | 2.71E−12 |
| CCR2 | 0.54259795 | 4.61E−12 |
| RBMS1 | 0.44123545 | 7.74E−12 |
| RPS9 | 0.02243035 | 1.28E−11 |
| TSPAN14 | 0.6339025 | 1.50E−11 |
| SULT1B1 | 1.1126722 | 2.58E−11 |
| MCTP2 | 1.8900664 | 3.85E−11 |
| EMB | 0.5234957 | 4.13E−11 |
| HSPB1 | 0.5421697 | 4.35E−11 |
| RAB10 | 0.33806282 | 4.49E−11 |
| CFLAR | 0.34268075 | 5.44E−11 |
| KIAA0930 | 0.49989277 | 6.12E−11 |
| MYL6 | 0.08600398 | 8.56E−11 |
| CDC42EP3 | 0.34722883 | 1.02E−10 |
| TNIP1 | 0.66114837 | 2.05E−10 |
| PRKCB | 0.29177785 | 2.33E−10 |
| NAA38 | 0.17931002 | 2.71E−10 |
| GAS7 | 0.51976514 | 3.04E−10 |
| PSMA7 | 0.08053571 | 3.08E−10 |
| RAB8B | 0.5691495 | 3.12E−10 |
| GYG1 | 0.52206373 | 3.17E−10 |
| COX6B1 | 0.01084459 | 3.54E−10 |
| C6orf62 | 0.35186508 | 3.60E−10 |
| LINC00152 | 0.36025366 | 4.15E−10 |
| NDUFB9 | 0.16023639 | 4.23E−10 |
| CCDC69 | 0.44219142 | 5.34E−10 |
| CPD | 0.7504521 | 5.80E−10 |
| RUNX1 | 0.76618445 | 6.68E−10 |
| YWHAG | 0.5551726 | 6.71E−10 |
| DDX17 | 0.23651825 | 6.91E−10 |
| F5 | 1.3194835 | 7.27E−10 |
| SNHG25 | 0.5180283 | 7.51E−10 |
| TOMM7 | 0.012599452 | 8.10E−10 |
| NBEAL2 | 0.6955884 | 1.34E−09 |
| MARCO | 0.54318714 | 1.39E−09 |
| LAMP1 | 0.42049694 | 1.43E−09 |
| PRR34-AS1 | 0.78917795 | 1.78E−09 |
| BATF | 0.7281571 | 1.85E−09 |
| GLUL | 0.27205062 | 2.22E−09 |
| PNPLA6 | 0.66430855 | 2.48E−09 |
| TCIRG1 | 0.40596047 | 2.49E−09 |
| ST20 | 0.95339906 | 3.09E−09 |
| PTPN12 | 0.536913 | 3.14E−09 |
| MAPK14 | 0.6136134 | 3.19E−09 |
| LILRA1 | 0.50736594 | 3.26E−09 |
| FLOT2 | 0.6100189 | 3.65E−09 |
| SLC16A3 | 0.33793104 | 4.06E−09 |
| SDCBP | 0.13519244 | 5.26E−09 |
| SHOC2 | 0.3754378 | 6.93E−09 |
| ARPC5 | 0.07265956 | 8.38E−09 |
| C3AR1 | 0.6360008 | 9.13E−09 |
| RASGRP2 | 0.35076612 | 1.04E−08 |
| PTK2B | 0.5779424 | 1.23E−08 |
| KCNE3 | 0.42077154 | 1.51E−08 |
| ATP6V1B2 | 0.28345326 | 1.53E−08 |
| MGST1 | 0.24790986 | 1.96E−08 |
| SLC16A7 | 0.9937738 | 1.97E−08 |
| WDR1 | 0.34414953 | 1.97E−08 |
| RTN3 | 0.28188503 | 2.35E−08 |
| LINC00482 | 1.0785195 | 2.40E−08 |
| BASP1 | 0.697361 | 2.88E−08 |
| SMPDL3A | 1.1846147 | 3.53E−08 |
| IGF2R | 0.7434028 | 4.13E−08 |
| JAK3 | 0.7367914 | 4.59E−08 |
| CRISPLD2 | 0.69640553 | 4.60E−08 |
| SGMS2 | 1.0042313 | 4.77E−08 |
| UQCRQ | 0.027639192 | 6.26E−08 |
| GLIPR2 | 0.20300446 | 6.36E−08 |
| ACTN4 | 0.52092314 | 6.67E−08 |
| OST4 | 0.017248146 | 6.95E−08 |
| NFAM1 | 0.5328003 | 8.64E−08 |
| IDH1 | 0.75986856 | 9.00E−08 |
| CREB5 | 0.470956 | 1.17E−07 |
| IL16 | 0.42563888 | 1.24E−07 |
| MZT2B | 0.14662817 | 1.24E−07 |
| PTPRJ | 0.6297723 | 1.66E−07 |
| MAPK1 | 0.38605338 | 1.78E−07 |
| NDUFB7 | 0.09155628 | 1.81E−07 |
| HSP90B1 | 0.31016114 | 2.00E−07 |
| IL10RB | 0.44816068 | 2.08E−07 |
| RNF144B | 0.4941323 | 2.65E−07 |
| SH3GLB1 | 0.325094 | 3.40E−07 |
| NAIP | 0.2845427 | 4.73E−07 |
| METTL7B | 2.1608682 | 4.83E−07 |
| EHD1 | 0.9658446 | 6.21E−07 |
| TNFRSF1B | 0.18429026 | 6.72E−07 |
| IGFBP2 | 2.2708569 | 6.90E−07 |
| PRKAR1A | 0.27777526 | 1.01E−06 |
| UBE2F | 0.43057647 | 1.07E−06 |
| LRRFIP1 | 0.12851237 | 1.11E−06 |
| WDR26 | 0.5436984 | 1.16E−06 |
| GNB1 | 0.35066724 | 1.21E−06 |
| CR1 | 0.9002841 | 1.42E−06 |
| G6PD | 0.40645602 | 2.35E−06 |
| MARCH1 | 0.20727344 | 2.66E−06 |
| FYB | 0.08225876 | 2.66E−06 |
| RP2 | 0.6375206 | 3.09E−06 |
| JDP2 | 0.78056455 | 3.92E−06 |
| PDIA4 | 0.60301614 | 4.15E−06 |
| TOP1 | 0.43470746 | 4.42E−06 |
| RPS24 | −0.000973117 | 4.63E−06 |
| RGS18 | 0.28991014 | 6.26E−06 |
| SASH3 | 0.42326063 | 7.91E−06 |
| STK38 | 0.46188563 | 8.44E−06 |
| BCL6 | 0.49014977 | 9.33E−06 |
| TIMP1 | 0.05811575 | 1.01E−05 |
| RPS6KA1 | 0.44873562 | 1.12E−05 |
| GM2A | 0.6874029 | 1.28E−05 |
| TLR8 | 0.56973124 | 1.30E−05 |
| NISCH | 0.7104525 | 1.38E−05 |
| RPS29 | 0.06588244 | 1.45E−05 |
| TRIP12 | 0.43021423 | 1.66E−05 |
| PPP1CB | 0.22190766 | 1.82E−05 |
| ATP13A3 | 0.74783397 | 2.08E−05 |
| FAM45A | 0.218513 | 2.40E−05 |
| MT-ND6 | 0.36885387 | 2.43E−05 |
| LAMTOR4 | −0.03467756 | 2.88E−05 |
| EIF4E3 | 0.5567325 | 2.90E−05 |
| NACC2 | 0.5140943 | 3.46E−05 |
| SLC25A37 | 0.341718 | 3.48E−05 |
| ENO1 | 0.074052826 | 3.51E−05 |
| FOSL2 | 0.4204945 | 3.80E−05 |
| CSGALNACT2 | 0.4479326 | 4.39E−05 |
| TNFAIP6 | 2.3433127 | 4.64E−05 |
| PLXNC1 | 0.4908287 | 4.97E−05 |
| CNIH4 | 0.28083116 | 7.08E−05 |
| RAB13 | 0.64946 | 7.11E−05 |
| FNDC3B | 0.55931664 | 7.95E−05 |
| CD99 | 0.101912916 | 7.95E−05 |
| CSF2RB | 0.46743047 | 8.53E−05 |
| FES | 0.4054935 | 9.46E−05 |
| PSTPIP1 | 0.35175237 | 0.000106683 |
| ADAM10 | 0.30670184 | 0.000114006 |
| FAR1 | 0.42571545 | 0.000114277 |
| HMOX1 | 0.3530429 | 0.000122731 |
| FAM107B | 0.41738078 | 0.000123645 |
| ATP6V1A | 0.36505392 | 0.000131709 |
| SRGN | 0.023015883 | 0.000137957 |
| VNN1 | 1.1805 | 0.000145791 |
| AIM1 | 0.535748 | 0.000147759 |
| ACSL4 | 0.511695 | 0.000149286 |
| EZR | 0.36933884 | 0.000161615 |
| DTX3L | 0.5960141 | 0.000161649 |
| FAM20A | 1.3786174 | 0.000169526 |
| LMNB1 | 0.6391043 | 0.000175256 |
| NPLOC4 | 0.6493585 | 0.000183402 |
| RRP12 | 0.62438464 | 0.000184426 |
| PAK2 | 0.2090441 | 0.000184564 |
| P4HB | 0.25392053 | 0.000187916 |
| PPDPF | −0.005053191 | 0.000190084 |
| CPEB4 | 0.5523164 | 0.000192823 |
| LILRB3 | 0.25871438 | 0.000204091 |
| KLHL2 | 0.8295296 | 0.000209752 |
| MCL1 | 0.106628545 | 0.000217002 |
| ADRBK1 | 0.2775691 | 0.000227678 |
| PROK2 | 1.1274799 | 0.000259281 |
| VAMP5 | 0.07048447 | 0.000300464 |
| MAFB | 0.21625733 | 0.000301974 |
| LTB4R | 0.43574023 | 0.000306544 |
| NCF1 | 0.07423708 | 0.000370357 |
| RRBP1 | 0.48280734 | 0.000403512 |
| SEC25A24 | 0.510095 | 0.000456638 |
| TMED8 | 0.8784822 | 0.000460563 |
| MT1E | 0.8589023 | 0.000462872 |
| BRI3 | −0.026777128 | 0.000552684 |
| GOLPH3 | 0.4038046 | 0.000560304 |
| GNG2 | 0.36641845 | 0.000595512 |
| DNAJC5 | 0.46699953 | 0.000656497 |
| CALM1 | 0.07962 | 0.000715678 |
| ATP6AP1 | 0.3542401 | 0.000743322 |
| MTHFD2 | 0.37341082 | 0.001105353 |
| FAM129B | 1.1131177 | 0.001125538 |
| RPN1 | 0.31605902 | 0.001274591 |
| SDF2L1 | 0.33770162 | 0.001286943 |
| RIN3 | 0.29692706 | 0.001401778 |
| LRP1 | 0.24149673 | 0.00148746 |
| POMP | 0.01379685 | 0.001514347 |
| CAB39 | 0.37304148 | 0.001520474 |
| MRPL41 | 0.1506235 | 0.001546939 |
| ATP2A2 | 0.5629077 | 0.001556622 |
| CD164 | 0.18064122 | 0.001587404 |
| ECE1 | 0.8535824 | 0.001696056 |
| CD82 | 0.97091395 | 0.001704493 |
| NUDT16 | 0.2324117 | 0.001712087 |
| EDEM3 | 0.51392835 | 0.001787124 |
| OAZ1 | −0.025662612 | 0.001889075 |
| C5AR1 | 0.31020564 | 0.002066081 |
| ZFAND5 | 0.14811042 | 0.002436335 |
| TAPBP | 0.19362076 | 0.002468225 |
| PIK3CD | 0.5507664 | 0.002801782 |
| EMILIN2 | 0.334624 | 0.00290243 |
| CTSZ | 0.09361586 | 0.002921022 |
| SAMHD1 | 0.06327006 | 0.002938606 |
| ASAP1 | 0.37841716 | 0.002961147 |
| EIF4EBP2 | 0.25343755 | 0.003019865 |
| DICER1 | 0.27376208 | 0.003683226 |
| UBE2J1 | 0.22254807 | 0.004015706 |
| DYNC1I2 | 0.311868 | 0.004065988 |
| MAN1A1 | 0.749566 | 0.004114131 |
| VMP1 | 0.060046766 | 0.004229893 |
| MBD2 | 0.27428994 | 0.004255347 |
| POLD4 | 0.31654742 | 0.004441262 |
| UNC13D | 0.6495286 | 0.004465846 |
| ADD3 | 0.29397976 | 0.00449781 |
| SLC12A6 | 0.4612941 | 0.00455543 |
| ARID1A | 0.29324776 | 0.004681124 |
| RHOU | 0.7333714 | 0.004703606 |
| MT1G | 2.5600884 | 0.004714535 |
| BCAT1 | 0.7670732 | 0.004751175 |
| MT-ATP8 | 0.50621825 | 0.004798493 |
| ASPH | 0.82223314 | 0.004907283 |
| TNIP3 | 4.254475 | 0.005046181 |
| MAP4K4 | 0.42832196 | 0.005324412 |
| SAP30 | 0.63528883 | 0.005399326 |
| MARCH1 | 0.71237254 | 0.00542617 |
| CDC42SE1 | 0.15264526 | 0.005615783 |
| RP11-84C10.2 | 1.0511802 | 0.005701224 |
| SPATA13 | 0.71142465 | 0.005947129 |
| METTL7A | 0.29000172 | 0.006701373 |
| SNX18 | 0.4083233 | 0.007307885 |
| TPM4 | 0.22038879 | 0.007743243 |
| HIPK1 | 0.44139567 | 0.007986633 |
| ETS2 | 0.4356731 | 0.009200953 |
| FURIN | 0.7280132 | 0.009435651 |
| P2RX1 | 0.7764762 | 0.009862631 |
| LTA4H | 0.046202738 | 0.010137063 |
| PLXND1 | 0.3840982 | 0.010469186 |
| SIRPA | 0.34314796 | 0.010696356 |
| PLEKHO2 | 0.4839532 | 0.010895049 |
| PCNX | 0.58932585 | 0.011516321 |
| RAB27A | 0.2635964 | 0.01164531 |
| UBA1 | 0.37780547 | 0.011906006 |
| HELZ | 0.3542133 | 0.012020539 |
| SIGLEC10 | 0.53294075 | 0.012634549 |
| RAB3D | 0.3043319 | 0.012827598 |
| CYSTM1 | 0.40797052 | 0.012855179 |
| TMEM2 | 0.62717265 | 0.013195513 |
| RBPJ | 0.20143872 | 0.013834445 |
| TMEM170B | 0.39153683 | 0.013898973 |
| APOBR | 0.33904767 | 0.014907167 |
| E2F2 | 1.5042969 | 0.014919493 |
| MANF | 0.48548132 | 0.014969696 |
| TPST2 | 0.36231193 | 0.015164829 |
| PLIN2 | 0.35985124 | 0.015164829 |
| IRF2BPL | 0.49433622 | 0.015845517 |
| VCL | 0.40262562 | 0.016415266 |
| TAF10 | 0.28006795 | 0.016764333 |
| FBXO9 | 0.355014 | 0.016936963 |
| CCR1 | 0.25716466 | 0.017375815 |
| NUP58 | 0.5219425 | 0.017925721 |
| UBR4 | 0.34273565 | 0.018221339 |
| TBCA | 0.041195195 | 0.018373183 |
| RTN4 | 0.0695505 | 0.019203881 |
| CEACAM4 | 0.4742593 | 0.019525631 |
| LIMS1 | 0.24951178 | 0.02089386 |
| FAM214B | 0.54299533 | 0.021076003 |
| CTNNA1 | 0.33827347 | 0.021669537 |
| SLC6A6 | 0.39036658 | 0.022304747 |
| ADAM9 | 0.67081875 | 0.022949429 |
| NFKB1 | 0.39033636 | 0.023261664 |
| LPGAT1 | 0.3035242 | 0.024740763 |
| CCDC167 | 0.50993204 | 0.026517587 |
| PTBP3 | 0.23654461 | 0.028084034 |
| MAP3K8 | 0.29336554 | 0.029277182 |
| ACTN1 | 0.38798374 | 0.029689604 |
| AGTRAP | 0.0477479 | 0.030646164 |
| TAF13 | 0.7367251 | 0.031779806 |
| CDK2AP2 | 0.23743378 | 0.031901234 |
| HSPA1A | 0.32994667 | 0.032592413 |
| MTMR3 | 0.56183594 | 0.033453845 |
| IL10RB-AS1 | 0.60315996 | 0.033491969 |
| HN1 | 0.16457497 | 0.036782787 |
| SPCS3 | 0.17753543 | 0.036939353 |
| HIPK3 | 0.264136 | 0.037506944 |
| C16orf72 | 0.29400447 | 0.0376335 |
| RHOG | 0.006316801 | 0.038042046 |
| SGTB | 0.6780799 | 0.038784242 |
| PDLIM7 | 0.44614542 | 0.039351379 |
| IL1RN | 0.4455267 | 0.04044708 |
| TM9SF2 | 0.18668634 | 0.041048472 |
| PADI4 | 0.6392817 | 0.041865934 |
| DOK3 | 0.30222762 | 0.042526845 |
| CDYL2 | 1.6434119 | 0.042697804 |
| PPM1M | 0.3186435 | 0.042968127 |
| ADAM17 | 0.3201454 | 0.043319632 |
| ATP1A1 | 0.28004715 | 0.043391533 |
| SH3BP2 | 0.23526847 | 0.043412782 |
| NDUFA11 | −0.02539604 | 0.043976153 |
| CPT1A | 0.4228959 | 0.044007859 |
| RHBDF2 | 0.38573143 | 0.044412374 |
| DPYD | 0.20450194 | 0.047482682 |
| NDRG1 | 0.6171073 | 0.047872429 |
| UBASH3B | 0.8465683 | 0.053227502 |
| N4BP1 | 0.58426774 | 0.054227768 |
| IL4R | 0.4692827 | 0.054227768 |
| WDFY3 | 0.5191001 | 0.054408186 |
| HBB | 1.9262346 | 0.057447168 |
| ARL4A | 0.32381642 | 0.057473287 |
| ACSL3 | 0.61024076 | 0.058035973 |
| SYK | 0.21143799 | 0.060385245 |
| SLC36A1 | 0.9914711 | 0.060767823 |
| CSNK1A1 | 0.16340353 | 0.063231556 |
| ASGR2 | 0.31869727 | 0.063856633 |
| HIP1 | 0.85026616 | 0.063977384 |
| ACER3 | 0.3235143 | 0.064285815 |
| TRABD | 0.2481931 | 0.064824405 |
| IL6ST | 0.42778534 | 0.071730473 |
| HRH2 | 0.2987029 | 0.072114254 |
| ABCA7 | 0.57369953 | 0.072114254 |
| FAM129A | 0.37902957 | 0.07276473 |
| WSB2 | 0.51066476 | 0.073640882 |
| LAIR1 | 0.27677563 | 0.07616532 |
| NLRC4 | 0.49999472 | 0.077025579 |
| TBC1D10B | 0.587827 | 0.077785011 |
| PADI2 | 0.7345606 | 0.080050891 |
| ITGAL | 0.24044655 | 0.081836663 |
| ADAMTS2 | 1.1353395 | 0.083407013 |
| MAP2K6 | 1.0804924 | 0.08378108 |
| MAP3K1 | 0.21254523 | 0.086417824 |
| RNF24 | 0.45556155 | 0.086929437 |
| NPTN | 0.27968496 | 0.087566061 |
| AGTPBP1 | 0.30352223 | 0.087566061 |
| KLF7 | 0.57635117 | 0.088175979 |
| MSL1 | 0.38628697 | 0.090262029 |
| MYO1G | 0.16193055 | 0.090516 |
| COX20 | 0.27356488 | 0.09124443 |
| RASSF3 | 0.1882066 | 0.092319696 |
| CORO1C | 0.25212872 | 0.093709216 |
| ACLY | 0.4116989 | 0.096049459 |
| PCYT1A | 0.4472556 | 0.097563192 |
| GSR | 0.515014 | 0.097639432 |
| AC004556.1 | 0.5176908 | 0.098686094 |
| HECA | 0.35043156 | 0.099143287 |

MS1 vs. MS4

| | | |
|---|---|---|
| PLAC8 | 2.6834667 | 0 |
| RETN | 2.77065 | 0 |
| CTSD | 2.0346358 | 0 |
| SELL | 1.992091 | 0 |
| CLU | 3.171919 | 0 |
| CYP1B1 | 2.1478531 | 0 |
| DUSP1 | 1.7316717 | 0 |
| CD36 | 1.7347965 | 0 |
| TIMP1 | 1.6462245 | 0 |
| MCEMP1 | 2.2647977 | 0 |
| ALOX5AP | 2.029591 | 0 |
| SOD2 | 1.7098211 | 0 |
| NAMPT | 2.064373 | 0 |
| RGS2 | 1.5128001 | 0 |
| CD163 | 2.4357023 | 0 |
| FAM65B | 1.4432725 | 0 |
| NKG7 | 1.3357148 | 4.58E−276 |
| CD99 | 1.2601565 | 5.55E−237 |
| ITGAM | 1.3763115 | 5.37E−209 |
| STXBP2 | 1.2288339 | 1.48E−204 |
| SLC11A1 | 1.1083503 | 5.34E−178 |
| ROMO1 | 1.0630426 | 6.74E−177 |
| TMEM176B | 1.351234 | 1.28E−175 |
| ACSL1 | 1.9117036 | 6.76E−167 |
| MT1X | 1.547267 | 1.36E−164 |
| ATOX1 | 1.0105096 | 5.33E−163 |
| CTSA | 1.2930936 | 1.04E−162 |
| VAMP5 | 1.0295805 | 5.85E−161 |
| IFI27L2 | 1.0064006 | 1.58E−159 |
| FLNA | 1.0026498 | 7.93E−156 |
| TUBA1B | 1.2024951 | 3.19E−155 |
| AHNAK | 1.0162011 | 2.39E−154 |
| NFKBIA | 1.2773824 | 1.74E−153 |
| COX17 | 0.97703695 | 3.16E−153 |
| CD93 | 1.1178768 | 5.62E−148 |
| MSRB1 | 1.0229114 | 1.80E−147 |
| FOS | 0.9971389 | 6.16E−147 |
| ANXA6 | 1.7012901 | 1.48E−146 |
| IL17RA | 1.2117969 | 1.07E−145 |
| VNN2 | 1.5561702 | 3.59E−141 |
| MPEG1 | 0.9417149 | 3.95E−140 |
| FAM198B | 1.376346 | 7.21E−140 |
| ATP6V1B2 | 1.0240076 | 1.01E−133 |
| DUSP6 | 0.9437985 | 2.58E−131 |
| KLF6 | 0.9042918 | 8.00E−131 |
| STAB1 | 1.2313107 | 1.63E−123 |
| NAIP | 1.0594059 | 3.61E−121 |
| RP6-159A1.4 | 1.0221155 | 4.07E−118 |
| SELPLG | 1.0593681 | 6.87E−117 |
| MYO1F | 0.8264199 | 1.69E−113 |
| SLC2A3 | 1.5932789 | 1.95E−111 |
| LINC00657 | 1.2387288 | 2.70E−109 |
| TAGLN2 | 0.8240124 | 1.05E−108 |
| CKAP4 | 1.676018 | 6.06E−107 |
| ZFP36 | 0.87713516 | 2.81E−106 |
| CFLAR | 0.89667165 | 7.90E−106 |
| LILRB3 | 0.99333245 | 8.89E−103 |
| SLC39A8 | 3.5078375 | 7.00E−102 |
| YWHAE | 0.8000871 | 1.66E−101 |
| C4orf48 | 0.9818137 | 1.58E−94 |
| EHBP1L1 | 0.95634085 | 3.07E−94 |
| TCIRG1 | 0.9743305 | 5.38E−93 |
| FLOT1 | 1.1663125 | 5.94E−93 |
| SOCS3 | 1.7801386 | 1.04E−92 |
| CES1 | 1.6340492 | 1.15E−92 |
| TAPBP | 0.8459693 | 4.42E−92 |
| FCGR3A | 1.2332549 | 5.15E−91 |
| CCDC69 | 1.0570109 | 3.00E−89 |
| PIM1 | 1.5513694 | 3.85E−89 |
| SDF2L1 | 1.1955146 | 7.46E−89 |
| HMOX1 | 1.2137146 | 1.55E−88 |
| DPYSL2 | 0.8178964 | 6.77E−88 |
| CALR | 0.787033 | 4.39E−86 |
| MT2A | 0.79519105 | 5.80E−86 |
| MS4A7 | 0.7936289 | 6.90E−84 |
| TMEM176A | 1.0584252 | 1.96E−83 |
| SYK | 1.1014276 | 1.69E−82 |
| RP11-295G20.2 | 1.0633569 | 4.86E−82 |
| LILRB1 | 1.0893308 | 1.79E−81 |
| CD300E | 0.9591577 | 2.67E−81 |
| C6orf62 | 0.76935095 | 6.67E−81 |
| GAS7 | 1.2053448 | 6.67E−81 |
| LBR | 1.1287249 | 1.70E−80 |
| CREB5 | 1.2706846 | 3.06E−80 |
| EMB | 1.135959 | 6.85E−80 |
| CDA | 0.7171759 | 1.78E−79 |
| ATP6AP1 | 1.2694225 | 6.56E−79 |
| LRP1 | 0.8465628 | 2.85E−77 |
| PRAM1 | 0.8247364 | 2.85E−77 |
| JAML | 0.6675858 | 2.89E−77 |
| CLEC12A | 0.68461984 | 1.21E−75 |
| ADAM10 | 0.9256516 | 3.89E−73 |
| NFE2 | 1.1585557 | 3.68E−71 |
| ZYX | 0.6279834 | 1.14E−69 |
| PSTPIP1 | 0.9789188 | 1.38E−68 |
| EIF4EBP2 | 0.852464 | 3.65E−68 |
| MXD1 | 1.2169302 | 6.14E−67 |
| H1FX | 0.7112568 | 2.05E−66 |
| LINC01272 | 0.64444655 | 1.19E−65 |
| CHCHD5 | 0.65015066 | 5.15E−65 |
| PLEC | 1.0004779 | 1.78E−64 |
| LRRK2 | 0.82815903 | 2.95E−64 |
| FAM26F | 0.7035289 | 3.61E−63 |
| ADRBK1 | 0.7424788 | 4.12E−63 |
| FAM200B | 0.71593577 | 1.46E−62 |
| AES | 0.7523766 | 4.88E−62 |
| FES | 1.0803131 | 7.29E−62 |
| OSCAR | 0.9051662 | 1.96E−61 |
| RIN3 | 0.8555301 | 1.98E−61 |
| ZSCAN16-AS1 | 0.809664 | 1.88E−60 |
| MT1F | 1.7451392 | 2.30E−60 |
| IL1R2 | 3.1961305 | 4.52E−60 |
| TRABD | 1.0057548 | 1.35E−59 |
| TNIP1 | 1.2461655 | 2.31E−59 |
| MYADM | 0.90469676 | 2.41E−59 |
| C19orf60 | 0.5640048 | 2.83E−58 |
| PSME2 | 0.5802871 | 1.69E−57 |
| LILRA5 | 0.5183792 | 3.16E−57 |
| SLC25A37 | 0.77162147 | 7.28E−57 |
| RXRA | 0.79596114 | 8.93E−57 |
| S100P | 2.0408256 | 2.76E−56 |
| IFITM3 | 0.6304251 | 7.22E−56 |
| RPS6KA1 | 1.0164689 | 3.36E−55 |
| MAP3K1 | 0.7808075 | 2.54E−54 |
| NCF4 | 0.91487545 | 4.37E−54 |
| GTF2I | 0.93364674 | 8.53E−53 |
| SLC38A2 | 0.90862185 | 2.19E−52 |
| NFAM1 | 0.98335785 | 2.44E−52 |
| PTK2B | 1.0412685 | 2.82E−52 |
| JUND | 0.55837643 | 1.21E−51 |
| SASH3 | 0.8787163 | 1.53E−51 |
| ARID1A | 0.846854 | 1.86E−51 |
| RNF144B | 0.91623163 | 4.35E−51 |
| ADGRE5 | 0.57952654 | 1.53E−50 |
| FAR1 | 1.0408695 | 7.75E−50 |
| STK38 | 0.9711871 | 8.01E−50 |
| THBS1 | 1.5754179 | 9.78E−50 |
| STK4 | 0.6618645 | 1.14E−49 |
| LTB4R | 1.176289 | 2.08E−49 |
| HSPA1A | 1.1068157 | 4.97E−49 |
| C17orf62 | 0.7108321 | 5.74E−49 |
| RP11-160E2.6 | 1.1712598 | 5.78E−49 |
| ALOX5 | 0.8556868 | 8.56E−48 |
| PTPN18 | 0.57926005 | 1.13E−47 |
| ZFP36L2 | 0.51149744 | 1.16E−47 |
| GOLPH3 | 1.0526196 | 1.68E−47 |
| SORL1 | 0.8063868 | 1.54E−46 |
| BATF | 1.2712046 | 1.86E−46 |
| PNPLA6 | 1.0565624 | 3.46E−46 |
| HMHA1 | 0.82039475 | 7.74E−46 |
| C16orfl3 | 0.51828825 | 1.80E−45 |
| MIF | 0.47595397 | 2.88E−45 |
| ISG15 | 0.5668362 | 3.65E−45 |
| LILRA6 | 1.1722548 | 5.83E−45 |
| APOBR | 1.0448743 | 5.89E−45 |
| UBR4 | 1.0160676 | 2.95E−44 |
| SIGIRR | 0.6980415 | 4.36E−44 |
| LAT2 | 0.70575464 | 8.09E−44 |
| FLOT2 | 0.89756703 | 8.33E−44 |
| POLR2I | 0.6133767 | 7.01E−43 |
| MZT2A | 0.83927774 | 7.13E−43 |
| BASP1 | 1.2813623 | 7.95E−43 |
| A1BG | 0.69483596 | 8.40E−43 |
| NOTCH2 | 0.62509114 | 8.58E−43 |
| AAK1 | 0.9384365 | 1.49E−42 |
| ASGR2 | 1.1177266 | 3.66E−42 |
| PDIA4 | 1.1526957 | 1.43E−41 |
| UBA1 | 1.0786086 | 1.66E−40 |
| LPXN | 0.9622928 | 6.56E−40 |
| HIPK3 | 0.74892956 | 8.18E−40 |
| TAF10 | 0.67272174 | 1.27E−39 |
| TUSC2 | 0.79053885 | 1.54E−39 |
| DDX3X | 0.4731858 | 2.84E−39 |
| ITGAL | 0.68694407 | 4.25E−39 |
| FMNL1 | 0.5037729 | 8.62E−39 |
| RNPEPL1 | 0.7417742 | 1.27E−38 |
| ATF4 | 0.53976303 | 1.70E−38 |
| NAPRT | 0.6393878 | 2.04E−38 |
| HSPB1 | 0.5732363 | 3.99E−38 |
| MOB3A | 0.73500216 | 4.72E−38 |
| PRMT2 | 0.5720945 | 8.67E−38 |
| RP11-347P5.1 | 0.56787217 | 8.72E−38 |
| SUN2 | 0.8952475 | 8.82E−38 |
| RUNX1 | 1.0718619 | 9.74E−38 |
| C16orf72 | 0.858061 | 1.86E−37 |
| CYTH4 | 0.76417863 | 7.66E−37 |
| SH3BP2 | 0.5696053 | 7.92E−37 |
| C1orf122 | 0.8122617 | 1.41E−36 |
| TRMT1 | 0.43425888 | 1.20E−35 |
| IRF2BP2 | 0.61623627 | 5.95E−35 |
| ACADVL | 0.7447258 | 6.71E−35 |
| MRPL55 | 0.60592204 | 7.33E−35 |
| GLTP | 0.78567207 | 7.47E−35 |
| NBEAL2 | 0.8367949 | 7.87E−35 |
| FAM195B | 0.5471023 | 1.19E−34 |
| CMIP | 0.6885152 | 1.82E−34 |
| CARD19 | 0.7615546 | 2.97E−34 |
| KCNAB2 | 0.9252677 | 5.03E−34 |
| STAT1 | 0.614953 | 9.66E−34 |
| C1QA | 1.2427158 | 1.21E−33 |
| CSF2RB | 0.8568763 | 2.97E−33 |
| MTRNR2L1 | 0.98261184 | 3.13E−33 |
| TMED2 | 0.42183015 | 3.13E−33 |
| ANPEP | 0.8273229 | 6.86E−33 |
| IST1 | 0.88308597 | 1.16E−32 |
| CECR1 | 0.48032027 | 1.30E−32 |
| CISD3 | 0.52020323 | 1.38E−32 |
| CXCR4 | 0.8851789 | 2.82E−32 |
| GCHFR | 0.78223175 | 5.65E−32 |
| NAA10 | 0.52481675 | 5.86E−32 |
| SHISA5 | 0.6923912 | 5.89E−32 |
| TES | 0.6759137 | 7.15E−32 |
| IGKC | 0.47523558 | 9.24E−32 |
| ARAP1 | 0.7930083 | 1.08E−31 |
| PPP6C | 0.84003097 | 1.13E−31 |
| MBOAT7 | 0.75061435 | 1.90E−31 |
| FBXL15 | 0.6049767 | 8.75E−31 |
| MAP3K11 | 0.80424535 | 1.28E−30 |
| STAG2 | 0.46701097 | 1.90E−30 |
| NOSIP | 0.5257825 | 2.05E−30 |
| RAB1B | 0.8508409 | 2.51E−30 |
| LY6E | 0.35156962 | 6.80E−30 |
| STARD7 | 0.7699389 | 8.25E−30 |
| DYSF | 1.342074 | 9.16E−30 |
| PRPF4B | 0.6564478 | 1.13E−29 |
| RTF1 | 0.5283223 | 1.68E−29 |
| SFC6A6 | 0.99518037 | 4.51E−29 |
| CRISPLD2 | 0.9530082 | 5.27E−29 |
| CARD8 | 0.5589405 | 9.40E−29 |
| TNFAIP2 | 0.37696618 | 9.67E−29 |
| FRCH4 | 0.690461 | 1.70E−28 |
| NME3 | 0.40636468 | 1.83E−28 |
| UQCC3 | 0.64906955 | 2.16E−28 |
| NDUFV3 | 0.5757249 | 2.41E−28 |
| SQSTM1 | 0.51052976 | 3.10E−28 |
| APOBEC3A | 0.79551655 | 7.42E−28 |
| PRKACA | 0.82228535 | 1.13E−27 |
| CPEB4 | 0.98285407 | 1.30E−27 |
| HIPK1 | 1.0033945 | 1.34E−27 |
| RRP12 | 1.144413 | 2.01E−27 |
| R3HDM4 | 0.70926464 | 2.16E−27 |
| STAT6 | 0.58589315 | 2.45E−27 |
| ZFP36L1 | 0.3518991 | 4.16E−27 |
| CR1 | 1.5406318 | 4.16E−27 |
| DNAJC4 | 0.54776305 | 5.31E−27 |
| PRR34-AS1 | 0.8614223 | 5.55E−27 |
| EIF5B | 0.48924354 | 7.02E−27 |
| SIPA1 | 0.87262976 | 1.42E−26 |
| OGFRE1 | 0.40827224 | 1.68E−26 |
| NISCH | 1.1459894 | 2.49E−26 |
| PEEKHJ1 | 0.5596552 | 2.63E−26 |
| PABPN1 | 0.42927626 | 3.47E−26 |
| IL1RN | 1.1679146 | 5.53E−26 |
| ACAP1 | 0.77884454 | 6.55E−26 |
| RHOC | 0.5405182 | 7.54E−26 |
| PXN | 0.89502144 | 8.95E−26 |
| PSMA3-AS1 | 0.5270844 | 9.67E−26 |
| HP | 2.0310574 | 1.11E−25 |
| UPF2 | 0.55705935 | 1.38E−25 |
| SLC12A6 | 0.9878098 | 1.38E−25 |
| MGEA5 | 0.5969189 | 1.44E−25 |
| ANKRD12 | 0.58465385 | 1.49E−25 |
| PLEKHO1 | 0.34201148 | 1.53E−25 |
| MYO9B | 0.6188371 | 1.56E−25 |
| GYPC | 0.62431103 | 2.36E−25 |
| CARS2 | 0.482421 | 2.60E−25 |
| FAM133B | 0.4213926 | 5.99E−25 |
| PDLIM7 | 1.0759916 | 8.98E−25 |
| MARCKS | 0.53281087 | 9.25E−25 |
| PIP4K2A | 0.70315087 | 1.25E−24 |
| NCF1 | 0.5551959 | 1.78E−24 |
| TRIM38 | 0.50063354 | 1.86E−24 |
| KIF22 | 0.42069593 | 2.16E−24 |
| ZC3H11A | 0.8415451 | 4.08E−24 |
| ANKRD44 | 0.5494843 | 4.28E−24 |
| JAK3 | 0.737065 | 5.46E−24 |
| PIK3CD | 1.0741184 | 6.46E−24 |
| LFNG | 0.6429084 | 8.07E−24 |
| JUNB | 0.34468403 | 1.53E−23 |
| BIRC2 | 0.48570004 | 2.17E−23 |
| PPP2R5C | 0.4724107 | 2.27E−23 |
| CRTAP | 0.40327635 | 2.32E−23 |
| TBL1XR1 | 0.6705532 | 2.82E−23 |
| NPLOC4 | 1.048685 | 3.08E−23 |
| CDC42SE2 | 0.38874373 | 3.43E−23 |
| AUP1 | 0.4039095 | 9.62E−23 |
| JARID2 | 0.8061349 | 1.05E−22 |
| ANKRD13D | 0.51226336 | 1.09E−22 |
| ELOF1 | 0.5178243 | 1.15E−22 |
| RASSF4 | 0.6551349 | 1.44E−22 |
| MX2 | 0.79295003 | 1.45E−22 |
| EHD1 | 1.2894934 | 2.09E−22 |
| HK3 | 0.76262885 | 2.31E−22 |
| RSBN1L | 0.8007653 | 4.27E−22 |
| CTBP1 | 0.50944865 | 4.61E−22 |
| CLK1 | 0.70192194 | 5.61E−22 |
| COLGALT1 | 0.77199197 | 1.35E−21 |
| TNNT1 | 1.0744644 | 1.46E−21 |
| PLXNB2 | 0.50587094 | 2.38E−21 |
| SEC61A1 | 0.9070997 | 2.73E−21 |
| YTHDC1 | 0.72175664 | 2.77E−21 |
| NDUFA7 | 0.4461855 | 2.94E−21 |
| MIIP | 0.5523462 | 3.11E−21 |
| GZMA | 0.6344292 | 4.03E−21 |
| RBP7 | 0.329803 | 4.40E−21 |
| OGT | 0.67183805 | 4.47E−21 |
| PLEKHA2 | 0.82875764 | 5.83E−21 |
| LMO4 | 0.5828197 | 6.01E−21 |
| RAE1 | 0.70400816 | 6.23E−21 |
| ADAM17 | 0.5796656 | 7.35E−21 |
| CST7 | 0.53643185 | 7.92E−21 |
| ARHGAP4 | 0.40966067 | 9.90E−21 |
| PHF21A | 1.0557847 | 1.23E−20 |
| DNAJB11 | 0.33521074 | 1.37E−20 |
| DPP7 | 0.35520387 | 1.92E−20 |
| ODF3B | 0.31579652 | 2.00E−20 |
| AGO4 | 0.78116715 | 2.00E−20 |
| CCDC107 | 0.6115242 | 2.00E−20 |
| AKNA | 0.52543736 | 2.25E−20 |
| PSMB8-AS1 | 0.38479906 | 3.05E−20 |
| STRA13 | 0.47892654 | 3.43E−20 |
| MSL1 | 0.90639 | 4.90E−20 |
| GIMAP8 | 0.8806021 | 8.43E−20 |
| FO538757.2 | 0.34834415 | 2.24E−19 |
| SMG1 | 0.6312613 | 2.72E−19 |
| PPM1F | 0.6591933 | 3.05E−19 |
| BOD1L1 | 0.68409646 | 3.16E−19 |
| MAP7D1 | 0.69311094 | 4.33E−19 |
| FOER3 | 0.49228308 | 4.43E−19 |
| XIST | 0.5081952 | 7.43E−19 |
| CBX6 | 0.49663585 | 8.41E−19 |
| CHIC2 | 0.66585916 | 1.05E−18 |
| SEC38A10 | 0.86783576 | 1.15E−18 |
| IAH1 | 0.44328228 | 1.26E−18 |
| RNF24 | 1.0979263 | 1.62E−18 |
| CREBRF | 0.7065177 | 2.92E−18 |
| NUP50 | 0.6930176 | 3.07E−18 |
| AP2B1 | 0.81870484 | 3.40E−18 |
| CMC1 | 0.67395586 | 3.97E−18 |
| CCE5 | 0.19524112 | 4.12E−18 |
| ARHGAP9 | 0.5046079 | 4.41E−18 |
| RASGRP4 | 0.7411826 | 4.96E−18 |
| YPEL3 | 0.20919648 | 5.80E−18 |
| FAM214B | 1.1091377 | 7.69E−18 |
| GZMB | 0.45572448 | 9.08E−18 |
| TNFRSF14 | 0.29346088 | 1.55E−17 |
| TMEM91 | 0.56337565 | 1.57E−17 |
| JDP2 | 0.79643565 | 1.70E−17 |
| PPM1B | 0.8289917 | 2.22E−17 |
| NRGN | 0.32860133 | 3.12E−17 |
| YIF1B | 0.6436086 | 3.80E−17 |
| KLF10 | 0.40998372 | 4.14E−17 |
| TLE3 | 0.6685309 | 4.48E−17 |
| PPP1R9B | 0.6511441 | 5.33E−17 |
| ZNF217 | 0.66197723 | 6.49E−17 |
| C12orf57 | 0.6041321 | 6.68E−17 |
| RNF167 | 0.5839906 | 9.87E−17 |
| TOM1 | 0.83647805 | 1.17E−16 |
| XPO1 | 0.672261 | 1.33E−16 |
| HIST1H4C | 0.7045354 | 1.49E−16 |
| FAM91A1 | 0.61984384 | 1.50E−16 |
| LENG8 | 0.25701344 | 1.61E−16 |
| LINC00482 | 0.9407084 | 1.62E−16 |
| ZBTB7B | 0.7709974 | 2.11E−16 |
| SDHAF2 | 0.7145882 | 2.51E−16 |
| EGLN2 | 0.5220857 | 2.94E−16 |
| SP3 | 0.61168325 | 4.91E−16 |
| IER2 | 0.20971465 | 5.12E−16 |
| IL4R | 0.9001733 | 5.40E−16 |
| SLC15A4 | 1.1662946 | 6.09E−16 |
| IL27RA | 0.7652713 | 6.78E−16 |
| SLCO3A1 | 0.64522284 | 8.74E−16 |
| CCM2 | 0.57851315 | 1.12E−15 |
| BRD4 | 0.41375583 | 1.16E−15 |
| IQSEC1 | 0.6755465 | 1.16E−15 |
| GSDMD | 0.38762844 | 1.22E−15 |
| TOPORS-AS1 | 0.5658863 | 1.22E−15 |
| KIAA2013 | 0.8194552 | 1.88E−15 |
| UNC13D | 0.94240135 | 1.90E−15 |
| IL2RG | 0.74099576 | 2.08E−15 |
| SAR1A | 0.38665807 | 2.19E−15 |
| SP1 | 0.7036779 | 2.26E−15 |
| DEF8 | 0.57622415 | 2.97E−15 |
| DNM2 | 0.6844709 | 3.26E−15 |
| STAT2 | 0.400525 | 3.54E−15 |
| SMARCD2 | 1.0190076 | 3.98E−15 |
| TRIM56 | 0.6030382 | 5.26E−15 |
| PLCB2 | 0.5012337 | 5.26E−15 |
| PDAP1 | 0.59602296 | 5.29E−15 |
| ROGDI | 0.50752115 | 8.35E−15 |
| ARHGAP27 | 0.5014642 | 9.44E−15 |
| ARRDC1 | 0.4059092 | 9.88E−15 |
| MKNK1 | 0.8076249 | 1.02E−14 |
| PNPLA2 | 0.717729 | 1.12E−14 |
| RNF166 | 0.3587348 | 1.29E−14 |
| YTHDF3 | 0.9360164 | 1.38E−14 |
| E2F3 | 1.203273 | 1.57E−14 |
| NUP58 | 0.90091914 | 1.65E−14 |
| GNLY | 0.27272052 | 1.65E−14 |
| ABTB1 | 0.3327824 | 1.72E−14 |
| IGHA1 | 1.8824328 | 1.82E−14 |
| PTPRA | 0.76984173 | 1.90E−14 |
| TPD52L2 | 0.54410934 | 2.49E−14 |
| OXLD1 | 0.42518333 | 2.64E−14 |
| C1orf228 | 0.58207226 | 3.22E−14 |
| KIAA0430 | 0.8064012 | 3.28E−14 |
| WDFY3 | 1.0703889 | 3.30E−14 |
| APBB3 | 0.6869016 | 4.11E−14 |
| NT5C | 0.33009365 | 4.27E−14 |
| ATP2B4 | 1.2344265 | 4.38E−14 |
| RP11-792A8.4 | 0.8884143 | 8.77E−14 |
| MGAT4A | 1.0038934 | 1.06E−13 |
| TNKS2 | 0.67409104 | 1.28E−13 |
| PADI4 | 1.2325085 | 1.37E−13 |
| ZC3HAV1 | 0.602467 | 2.02E−13 |
| TRIOBP | 0.606497 | 2.20E−13 |
| ELAVL1 | 0.5305411 | 2.29E−13 |
| FUBP1 | 0.6633258 | 2.55E−13 |
| C19orf25 | 0.56898814 | 2.85E−13 |
| CSF2RA | 0.4154703 | 3.02E−13 |
| NSRP1 | 0.54343015 | 3.36E−13 |
| PLXND1 | 0.31852397 | 5.87E−13 |
| AP1M1 | 0.73680824 | 8.31E−13 |
| ITGA5 | 0.89740586 | 9.63E−13 |
| FOSL2 | 0.19676304 | 1.01E−12 |
| IL32 | 0.09504308 | 1.27E−12 |
| UBE2M | 0.8314881 | 1.27E−12 |
| SCO2 | 0.15892753 | 1.37E−12 |
| RHBDF2 | 0.38516402 | 1.44E−12 |
| SPATA13 | 1.0594103 | 1.48E−12 |
| SERPINB9 | 0.2723551 | 1.79E−12 |
| KDM3B | 0.83854586 | 2.11E−12 |
| ECE1 | 1.2641685 | 2.12E−12 |
| METTL22 | 0.61971194 | 2.41E−12 |
| DYNC1LI2 | 0.86630344 | 2.64E−12 |
| VAV1 | 0.872592 | 2.66E−12 |
| IRAK1 | 0.832621 | 2.89E−12 |
| GZMH | 0.5543912 | 2.95E−12 |
| TUBA4A | 0.67301947 | 3.34E−12 |
| RP5-940J5.9 | 0.54972225 | 3.60E−12 |
| BAD | 0.60774463 | 3.82E−12 |
| ADAM15 | 0.55295134 | 4.26E−12 |
| MAN2A1 | 0.7891253 | 4.35E−12 |
| ZSWIM6 | 0.7930644 | 4.57E−12 |
| NARF | 0.68191093 | 4.92E−12 |
| BTG2 | 0.49801284 | 5.32E−12 |
| IRF5 | 0.7521698 | 5.40E−12 |
| POLR2J3 | 0.2611916 | 6.97E−12 |
| BAG6 | 0.6065253 | 7.77E−12 |
| SLC12A9 | 0.7196398 | 7.97E−12 |
| C1QB | 1.2337304 | 8.03E−12 |
| GAK | 0.69308335 | 8.74E−12 |
| CRLF3 | 0.65282446 | 1.13E−11 |
| MAZ | 0.5487565 | 1.13E−11 |
| PQLC1 | 1.0176173 | 1.14E−11 |
| IRAK4 | 0.90190285 | 1.55E−11 |
| ARF6 | 0.2045846 | 2.24E−11 |
| RPS6KA4 | 0.53829634 | 2.26E−11 |
| NXF1 | 0.6550532 | 2.73E−11 |
| MICAL1 | 0.70112026 | 2.81E−11 |
| STX16 | 0.7537622 | 2.92E−11 |
| SSBP4 | 0.27711698 | 2.96E−11 |
| RREB1 | 0.61136055 | 3.02E−11 |
| PPP6R1 | 0.9091684 | 3.28E−11 |
| PPP1CB | 0.1342544 | 3.31E−11 |
| SMNDC1 | 0.6406431 | 3.49E−11 |
| PRDM2 | 0.7826206 | 3.53E−11 |
| FURIN | 1.0309763 | 3.88E−11 |
| TRPS1 | 0.5611079 | 4.20E−11 |
| CCDC57 | 0.39782044 | 4.27E−11 |
| ADAP1 | 0.8482175 | 4.61E−11 |
| TMEM154 | 0.32845354 | 4.72E−11 |
| PRRC2B | 0.84202486 | 4.80E−11 |
| C15orf39 | 0.51541823 | 5.99E−11 |
| CH17-373J23.1 | 0.7783506 | 6.95E−11 |
| NSF | 1.0357789 | 7.09E−11 |
| SOX4 | 0.35872045 | 7.58E−11 |
| SMG7 | 0.67802197 | 8.07E−11 |
| SLC9A3R1 | 0.46093515 | 8.68E−11 |
| POLD4 | 0.119488664 | 9.18E−11 |
| DENND4B | 0.54955876 | 9.56E−11 |
| HOMER3 | 0.766922 | 9.65E−11 |
| RHOB | 0.39022234 | 1.05E−10 |
| ZNF385A | 0.2756389 | 1.08E−10 |
| DUSP22 | 0.3931221 | 1.18E−10 |
| TNRC6A | 0.7043031 | 1.29E−10 |
| TIA1 | 0.7850413 | 1.29E−10 |
| TBC1D10B | 0.9873541 | 1.31E−10 |
| CCDC88B | 0.45411736 | 1.33E−10 |
| NLRP12 | 0.9193356 | 1.33E−10 |
| CDK12 | 0.80812925 | 1.33E−10 |
| FGD3 | 0.36269292 | 1.37E−10 |
| C6orf1 | 0.35813272 | 1.37E−10 |
| EFTUD2 | 1.0220702 | 1.42E−10 |
| HOPX | 0.91254693 | 1.71E−10 |
| GRIPAP1 | 0.8091994 | 1.84E−10 |
| ESRRA | 0.054839596 | 1.87E−10 |
| RAP2B | 0.16252716 | 1.91E−10 |
| WDR13 | 0.68847615 | 2.02E−10 |
| IRF7 | 0.32517117 | 2.49E−10 |
| POR | 0.97301847 | 2.69E−10 |
| RBM23 | 0.4828926 | 2.71E−10 |
| CSF3R | 0.16485316 | 2.71E−10 |
| PRRC2A | 0.7863168 | 2.82E−10 |
| C9orf142 | 0.051274374 | 2.96E−10 |
| HDAC7 | 0.9325536 | 3.04E−10 |
| FAM134A | 0.42448652 | 3.25E−10 |
| NCLN | 1.0075372 | 3.90E−10 |
| SLC25A1 | 0.73706126 | 4.09E−10 |
| UBE2W | 0.48048976 | 5.38E−10 |
| DYNLL2 | 0.9355048 | 5.38E−10 |
| CLN3 | 0.6805768 | 6.05E−10 |
| STEAP4 | 1.1756407 | 6.08E−10 |
| DOCK5 | 0.20282796 | 6.23E−10 |
| LLNLR-245B6.1 | 0.41047007 | 6.50E−10 |
| UNCI19 | 0.35947576 | 6.84E−10 |
| RHBDD2 | 0.84871453 | 6.87E−10 |
| RBBP4 | 0.10104052 | 7.15E−10 |
| TMEM259 | 0.39996827 | 7.91E−10 |
| AP3M1 | 0.8321581 | 8.26E−10 |
| IGLC2 | 0.8225483 | 9.69E−10 |
| RAB5B | 0.65313375 | 1.07E−09 |
| DEF6 | 0.5848348 | 1.07E−09 |
| SPSB3 | 0.26430318 | 1.07E−09 |
| FRMD4B | 0.66316926 | 1.22E−09 |
| LPCAT1 | 0.62693477 | 1.42E−09 |
| TBL1X | 0.8044429 | 1.61E−09 |
| CHD3 | 0.82343316 | 1.89E−09 |
| CHMP1A | 0.6118545 | 2.03E−09 |
| SULF2 | 0.27248332 | 2.03E−09 |
| MKL1 | 0.79296744 | 2.50E−09 |
| UBP1 | 0.8394125 | 2.50E−09 |
| MTHFR | 0.5708239 | 2.89E−09 |
| KIAA0319L | 0.66379535 | 2.89E−09 |
| MXD4 | 0.4925643 | 2.91E−09 |
| CD79B | 0.4179401 | 3.12E−09 |
| PLEKHF2 | 0.76206267 | 3.27E−09 |
| KIAA0100 | 0.7724873 | 3.34E−09 |
| CORO7 | 0.6059336 | 3.38E−09 |
| PARP8 | 0.7360362 | 3.43E−09 |
| ZSWIM8 | 0.8984899 | 3.55E−09 |
| CCDC159 | 0.47042498 | 3.70E−09 |
| PRDX2 | 0.3354391 | 4.57E−09 |
| KAT8 | 0.7118581 | 4.58E−09 |
| MECP2 | 0.4812527 | 4.66E−09 |
| CSRP1 | 0.7541688 | 5.24E−09 |
| TRIM13 | 0.7196415 | 5.42E−09 |
| FBXW11 | 0.7768585 | 5.67E−09 |
| SNHG25 | 0.101848766 | 5.95E−09 |
| INPPL1 | 0.6562067 | 6.07E−09 |
| CCS | 0.4357077 | 6.99E−09 |
| TTC7A | 0.68381727 | 7.43E−09 |
| TET3 | 0.71062046 | 8.00E−09 |
| PCSK7 | 0.3445853 | 8.72E−09 |
| YKT6 | 0.92643934 | 1.03E−08 |
| MAVS | 0.73952866 | 1.15E−08 |
| SLC35C2 | 0.83779573 | 1.18E−08 |
| SGK1 | 0.36006275 | 1.40E−08 |
| SUGP2 | 0.7497034 | 1.45E−08 |
| BRD9 | 0.7590861 | 1.47E−08 |
| RCHY1 | 0.89158255 | 1.52E−08 |
| ZKSCAN1 | 0.69731945 | 1.57E−08 |
| RICTOR | 0.61811733 | 1.79E−08 |
| MAST3 | 0.6147786 | 1.81E−08 |
| CD300LF | 0.35619622 | 2.01E−08 |
| SLC20A1 | 0.52138174 | 2.09E−08 |
| FOXP1 | 0.13672335 | 2.35E−08 |
| STX4 | 0.55800825 | 2.39E−08 |
| DCTN1 | 0.88307583 | 2.49E−08 |
| FAM160A2 | 0.90251553 | 2.61E−08 |
| NDUFAF2 | 0.50659883 | 2.62E−08 |
| KLRB1 | 0.23856437 | 2.71E−08 |
| RFC1 | 0.5022843 | 2.77E−08 |
| CLMN | 0.9355381 | 2.89E−08 |
| P2RX1 | 0.7199983 | 3.28E−08 |
| GDI1 | 0.66908914 | 4.59E−08 |
| ACBD3 | 0.7839196 | 4.60E−08 |
| TSPAN3 | 0.8049216 | 4.98E−08 |
| RC3H1 | 0.35288653 | 5.94E−08 |
| STK40 | 0.5775281 | 6.08E−08 |
| ZFAND2B | 0.35825706 | 6.39E−08 |
| ABCA7 | 0.60963976 | 6.84E−08 |
| RAP2C | 0.54515284 | 6.85E−08 |
| DEDD2 | 0.23955257 | 6.92E−08 |
| CPNE1 | 0.42248997 | 7.02E−08 |
| SNX30 | 0.5944641 | 7.43E−08 |
| GRAMD1A | 0.56867254 | 7.48E−08 |
| PBX2 | 0.50650823 | 7.50E−08 |
| AP2A2 | 0.7786971 | 7.86E−08 |
| BMP2K | 0.65993273 | 7.93E−08 |
| TLR5 | 0.89231676 | 7.95E−08 |
| TBC1D10C | 0.1785875 | 8.12E−08 |
| DPM2 | 0.4715767 | 8.47E−08 |
| FASTK | 0.43423304 | 8.65E−08 |
| KDM6B | 0.7168581 | 9.26E−08 |
| GTF2F1 | 0.47786546 | 9.42E−08 |
| SEPT1 | 0.23111802 | 1.01E−07 |
| NUMA1 | 0.7817343 | 1.30E−07 |
| RNF220 | 0.5310398 | 1.32E−07 |
| TMUB2 | 0.5005538 | 1.50E−07 |
| ITGAX | 0.06927787 | 1.54E−07 |
| CACNA2D4 | 0.62290996 | 1.70E−07 |
| MED15 | 0.66812694 | 1.85E−07 |
| XPO6 | 0.8960693 | 1.91E−07 |
| NLRC5 | 0.5219385 | 2.01E−07 |
| AC004556.1 | 0.42420965 | 2.08E−07 |
| CYHR1 | 0.45531207 | 2.09E−07 |
| ATP6V0A1 | 0.5797923 | 2.37E−07 |
| SET | 0.076938145 | 2.39E−07 |
| DCUN1D1 | 0.48288098 | 2.41E−07 |
| SNRNP200 | 0.6067696 | 2.48E−07 |
| SEC16A | 0.9298096 | 2.53E−07 |
| VSTM1 | 0.60406387 | 2.55E−07 |
| ORAI1 | 0.35196286 | 2.93E−07 |
| COASY | 0.6544136 | 2.95E−07 |
| TYK2 | 0.38842767 | 3.30E−07 |
| MT-ATP8 | 0.2327312 | 3.33E−07 |
| CD4 | 0.09655697 | 3.34E−07 |
| ZMAT3 | 0.7235216 | 3.35E−07 |
| CHTF8 | 0.5270829 | 3.73E−07 |
| CPSF6 | 0.66382277 | 3.77E−07 |
| CATSPER1 | 0.3665319 | 3.91E−07 |
| NAA60 | 0.6272799 | 4.17E−07 |
| SLC30A7 | 0.5343614 | 4.17E−07 |
| EVL | 0.13739526 | 4.70E−07 |
| NCOA2 | 0.56621075 | 5.51E−07 |
| WWP2 | 0.7923854 | 5.53E−07 |
| ANKRD28 | 1.2237598 | 5.72E−07 |
| ADNP | 0.5592513 | 5.86E−07 |
| PELI1 | 0.5735193 | 6.30E−07 |
| SEC23A | 0.65320325 | 6.31E−07 |
| GEMIN7 | 0.40238056 | 6.45E−07 |
| CAMTA2 | 0.67875004 | 6.65E−07 |
| UBA6 | 0.70093745 | 7.50E−07 |
| RP11-140K17.3 | 0.5766119 | 7.69E−07 |
| AKT2 | 0.7328855 | 8.20E−07 |
| POLDIP3 | 0.8753455 | 9.73E−07 |
| TTC39C | 0.56720227 | 1.09E−06 |
| ADAM8 | 0.7268559 | 1.09E−06 |
| PANK3 | 0.7355692 | 1.25E−06 |
| HSPH1 | 0.8551949 | 1.34E−06 |
| IGFLR1 | 0.39616004 | 1.46E−06 |
| RGS14 | 0.51618403 | 1.58E−06 |
| NADSYN1 | 0.56113523 | 1.99E−06 |
| U2AF2 | 0.6156756 | 2.02E−06 |
| SH3D19 | 0.49670643 | 2.11E−06 |
| USP25 | 0.6715336 | 2.15E−06 |
| TOMM5 | 0.29427063 | 2.30E−06 |
| TP53INP1 | 0.61681104 | 2.34E−06 |
| MAPKAPK5-AS1 | 0.4610944 | 2.34E−06 |
| TRIM26 | 0.8805508 | 2.38E−06 |
| GFER | 0.31063545 | 2.45E−06 |
| CENPB | 0.7995479 | 2.45E−06 |
| FAM134C | 0.72803044 | 2.49E−06 |
| UBXN11 | −0.023570064 | 2.49E−06 |
| PPP1R12C | 0.5488019 | 2.63E−06 |
| SUDS3 | 0.65499985 | 2.65E−06 |
| TSTD1 | 0.25895834 | 2.67E−06 |
| EXOC3 | 0.75233924 | 2.78E−06 |
| HGS | 0.57798284 | 2.82E−06 |
| CLCN7 | 0.55828875 | 2.84E−06 |
| TRAC | 0.23435159 | 2.90E−06 |
| CSNK1G2 | 0.6283965 | 3.19E−06 |
| KLRD1 | 0.5311994 | 3.19E−06 |
| EAF1 | 0.6765994 | 3.25E−06 |
| PTOV1 | 0.5351354 | 3.27E−06 |
| TFE3 | 0.65742505 | 3.44E−06 |
| SMARCD3 | 0.29326925 | 3.57E−06 |
| MT1G | 4.4529457 | 3.57E−06 |
| PIEZO1 | 0.6776037 | 3.85E−06 |
| ARHGEF40 | 0.66015434 | 3.89E−06 |
| PPP6R3 | 0.72201556 | 4.14E−06 |
| PGGT1B | 0.48678106 | 4.17E−06 |
| TMBIM1 | 0.6737972 | 4.26E−06 |
| LINC01506 | 0.52511775 | 4.38E−06 |
| STARD3 | 0.75225765 | 4.49E−06 |
| GPAT4 | 0.8511174 | 4.56E−06 |
| SPPL3 | 0.6372415 | 4.88E−06 |
| TNRC6B | −0.028313538 | 4.94E−06 |
| C19orf66 | 0.46912038 | 4.98E−06 |
| SBNO2 | 0.731777 | 5.12E−06 |
| PIKFYVE | 0.7071819 | 5.38E−06 |
| FBXO6 | 0.41126436 | 5.41E−06 |
| HPS3 | 0.68319464 | 5.54E−06 |
| C18orf32 | 0.34461972 | 5.79E−06 |
| RIN2 | 0.24680346 | 6.19E−06 |
| UBXN7 | 0.7530902 | 6.52E−06 |
| TRBC1 | 0.28720847 | 6.76E−06 |
| SYMPK | 0.61846215 | 7.53E−06 |
| CCL4 | 0.347525 | 7.90E−06 |
| CD3D | 0.069191694 | 8.41E−06 |
| NUP210 | 0.7574284 | 8.84E−06 |
| MRPL53 | 0.08617783 | 9.28E−06 |
| PSD4 | 0.56651694 | 9.92E−06 |
| APEH | 0.59867185 | 1.03E−05 |
| CIDEB | 0.7461324 | 1.08E−05 |
| PCIF1 | 0.5135458 | 1.14E−05 |
| CDK5RAP3 | 0.219232 | 1.16E−05 |
| UBAP2L | 0.7416719 | 1.20E−05 |
| TECPR1 | 0.78309333 | 1.26E−05 |
| COX16 | 0.35786182 | 1.27E−05 |
| OTUD1 | 0.61212915 | 1.27E−05 |
| ADAM19 | 0.67896616 | 1.30E−05 |
| OAS2 | 0.56939644 | 1.32E−05 |
| CD7 | 0.24792206 | 1.33E−05 |
| CHSY1 | 0.8612627 | 1.37E−05 |
| MARK2 | 0.5812409 | 1.39E−05 |
| EML3 | 0.56721956 | 1.40E−05 |
| ARSA | 0.70756775 | 1.41E−05 |
| EBLN3 | 0.56312263 | 1.41E−05 |
| RAPGEF1 | 0.7008794 | 1.49E−05 |
| YY1AP1 | 0.80620736 | 1.51E−05 |
| WASH1 | 0.2084945 | 1.54E−05 |
| ISG20 | 0.23109776 | 1.55E−05 |
| ATG16L2 | 0.055579133 | 1.57E−05 |
| STRN4 | 0.70826024 | 1.61E−05 |
| ARFRP1 | 0.698352 | 1.79E−05 |
| GPR27 | 1.2702138 | 1.87E−05 |
| STK11 | 0.6954611 | 1.94E−05 |
| DNAJB1 | 0.58353645 | 2.02E−05 |
| RNF38 | 0.7418893 | 2.08E−05 |
| RHOT2 | 0.3831127 | 2.08E−05 |
| ATP2A3 | 0.6340826 | 2.14E−05 |
| CXXC1 | 0.74395084 | 2.18E−05 |
| TMEM144 | 0.84032273 | 2.21E−05 |
| IPMK | 0.72819203 | 2.31E−05 |
| BCL3 | 0.6319185 | 2.49E−05 |
| ZNF451 | 0.58263594 | 2.52E−05 |
| MB21D1 | 0.75275165 | 2.56E−05 |
| TLK1 | 0.39960718 | 2.74E−05 |
| FBXW7 | 0.58763844 | 2.97E−05 |
| SH2D3C | 0.51601374 | 2.99E−05 |
| CDK19 | 0.6814421 | 3.02E−05 |
| AP1G1 | 0.23385176 | 3.24E−05 |
| BAK1 | 0.76941186 | 3.37E−05 |
| PPP3CA | 0.008295 | 3.40E−05 |
| NMT1 | 0.18587588 | 3.49E−05 |
| IFI6 | −0.10420767 | 3.74E−05 |
| TTC19 | 0.31561723 | 3.84E−05 |
| HAUS4 | 0.47681552 | 4.19E−05 |
| CIC | 0.8387368 | 4.40E−05 |
| RASSF1 | 0.22011963 | 4.41E−05 |
| HDAC10 | 0.5214664 | 4.48E−05 |
| ITCH | 0.6381829 | 4.60E−05 |
| SIGLEC14 | 0.15015683 | 4.80E−05 |
| SPG7 | 0.5550288 | 4.93E−05 |
| ATG4B | 0.47115615 | 5.30E−05 |
| ADAMTSL4 | 0.5203508 | 5.43E−05 |
| SCAMP3 | 0.70661587 | 5.71E−05 |
| PHKG2 | 0.36352825 | 6.05E−05 |
| SATB1 | 0.7151742 | 6.22E−05 |
| DUS1L | 0.51133794 | 6.66E−05 |
| PLPPR2 | 0.5737655 | 6.79E−05 |
| RNASE1 | 1.4209365 | 6.82E−05 |
| PDS5B | 0.46677303 | 7.26E−05 |
| TBC1D2B | 0.5339106 | 7.39E−05 |
| ITGB2-AS1 | 0.014542621 | 8.33E−05 |
| MNT | 0.6790764 | 8.38E−05 |
| TIAM1 | 0.51111233 | 8.46E−05 |
| DMAP1 | 0.6547334 | 9.25E−05 |
| TRG-AS1 | 0.5503077 | 9.42E−05 |
| MSL2 | 0.41313624 | 9.95E−05 |
| MEFV | 0.46095327 | 0.000114218 |
| SMCR5 | 0.5728581 | 0.000115089 |
| OTUD5 | 0.7038951 | 0.000117189 |
| LMF2 | 0.49285933 | 0.00012022 |
| NLRP1 | 0.30257776 | 0.000120377 |
| PPP6R2 | 0.4241599 | 0.000125162 |
| BCL10 | 0.3466628 | 0.00012943 |
| SETD5 | 0.2475532 | 0.000130232 |
| TRPC4AP | 0.44354898 | 0.000137765 |
| DENND1A | 0.5085375 | 0.000139191 |
| FHOD1 | 0.63555783 | 0.000141367 |
| ST3GAL5 | 0.18413518 | 0.000142387 |
| AREL1 | 0.8488993 | 0.000152094 |
| R3HDM2 | 0.02201509 | 0.000169403 |
| ARRDC4 | 0.5832401 | 0.000169526 |
| MFSD12 | 0.5087029 | 0.000174359 |
| KLC1 | 0.61973286 | 0.000176705 |
| EGR1 | 0.25108516 | 0.00017945 |
| PITPNM1 | 0.51337475 | 0.000179571 |
| TNIP3 | 3.680077 | 0.000179628 |
| ATXN7 | 0.2803438 | 0.000182411 |
| DLGAP4 | 0.63849765 | 0.000185292 |
| TRBC2 | −0.040573847 | 0.00020594 |
| JCHAIN | 0.97229105 | 0.000207035 |
| RP11-670E13.6 | 0.48196837 | 0.000212658 |
| TAZ | 0.56006765 | 0.000214158 |
| C2orf68 | −0.029091818 | 0.000222131 |
| CCNL2 | 0.4782201 | 0.000242044 |
| RAP1GAP2 | 0.58684176 | 0.000242044 |
| RELA | 0.6104448 | 0.000272104 |
| PBXIP1 | 0.6808811 | 0.000306199 |
| GPR141 | 1.1539551 | 0.00031543 |
| RBM6 | −0.015457449 | 0.000319848 |
| SYNGR1 | 0.39424884 | 0.000321842 |
| CD2 | 0.48306257 | 0.000342915 |
| ZDHHC2 | 0.6104386 | 0.000350497 |
| TANGO2 | 0.48283455 | 0.000368789 |
| XAF1 | 0.25360784 | 0.000373112 |
| NSUN5 | 0.38373962 | 0.000389086 |
| SMG5 | 0.6129891 | 0.000390717 |
| NPEPL1 | 0.44918957 | 0.000414177 |
| PIK3R5 | 0.524189 | 0.000422985 |
| STT3A | 0.91367626 | 0.000438462 |
| RBM10 | 0.6422785 | 0.000444243 |
| HSPA4 | 0.26177162 | 0.000456384 |
| ULK1 | 0.708369 | 0.000480709 |
| VPS9D1 | 0.7233937 | 0.000541155 |
| SLC40A1 | 0.5943221 | 0.000541155 |
| SYTL1 | 0.17964646 | 0.000575809 |
| TMC8 | 0.2937753 | 0.000582426 |
| C7orf49 | 0.4707841 | 0.00060434 |
| ZNF516 | 0.32223693 | 0.00061987 |
| GGA2 | 0.5292493 | 0.000674307 |
| DDX54 | 0.58724207 | 0.000705327 |
| MYLIP | 0.5882834 | 0.000705673 |
| SDR39U1 | 0.39630336 | 0.000705673 |
| ANXA2R | 0.32732764 | 0.000726906 |
| MAN2C1 | 0.3153123 | 0.000751147 |
| CEPT1 | 0.111467175 | 0.000753257 |
| TNK2 | 0.4552313 | 0.000756232 |
| SNHG15 | 0.3143031 | 0.000765045 |
| ORAI2 | 0.33810323 | 0.000824603 |
| HDAC5 | 0.5672603 | 0.000858326 |
| RP11-802E16.3 | 0.3397839 | 0.00087114 |
| XPC | 0.56531096 | 0.000943572 |
| IER3 | 0.10785521 | 0.000949662 |
| SEC24C | 0.6074529 | 0.000956246 |
| PARP10 | 0.4097565 | 0.000957451 |
| COQ4 | 0.3641489 | 0.000966494 |
| PPP3R1 | 0.008331252 | 0.000978752 |
| SERINC5 | 0.2051255 | 0.000991378 |
| DAXX | 0.56606674 | 0.000991378 |
| KIAA0368 | 0.62371796 | 0.001013981 |
| MDM4 | −0.09859166 | 0.001018767 |
| SLC44A2 | 0.3480641 | 0.001035778 |
| AP1G2 | 0.27201557 | 0.001097978 |
| CD3E | 0.1064087 | 0.001124602 |
| IFFO1 | 0.4493897 | 0.001164398 |
| RPUSD3 | 0.3638935 | 0.001164398 |
| ZNF264 | 0.6018032 | 0.001169178 |
| ZNF316 | 0.5479321 | 0.00118274 |
| ZNF302 | −0.12236149 | 0.001291129 |
| CHKB | 0.3958148 | 0.00132474 |
| C11orf57 | 0.44702053 | 0.001404967 |
| UBA7 | 0.3186705 | 0.0014242 |
| C3orf62 | 0.7906719 | 0.001430454 |
| TRAPPC12 | 0.46994257 | 0.00145348 |
| ARFGAP1 | 0.7315342 | 0.001496546 |
| RRP36 | 0.7031815 | 0.001593916 |
| RHOF | 0.13425168 | 0.001611328 |
| ASB1 | 0.8242321 | 0.001643682 |
| ANKRD10 | 0.14864495 | 0.001713686 |
| ACAP3 | 0.5433796 | 0.001716754 |
| RFX1 | 0.44230935 | 0.001780202 |
| ANKRD40 | 0.3671807 | 0.001828951 |
| ALYREF | 0.3726403 | 0.001860706 |
| CCDC117 | 0.5183001 | 0.001934776 |
| TRAFD1 | 0.5387689 | 0.001989326 |
| PAN3 | 0.039709542 | 0.002000037 |
| ZRSR2 | 0.14211698 | 0.002197579 |
| CALCOCO1 | 0.2896278 | 0.002526496 |
| PADI2 | 0.30284885 | 0.00253428 |
| DCAF8 | 0.4870931 | 0.00255292 |
| NDST1 | 0.7564376 | 0.002622636 |
| BTBD2 | 0.63956577 | 0.002645684 |
| TBC1D9 | 0.2608835 | 0.002683949 |
| SAP30L | 0.8843364 | 0.002701566 |
| FKBP4 | 0.5279831 | 0.002819361 |
| ELMO1 | 0.44525978 | 0.002993376 |
| RP11-15819.8 | 0.6146859 | 0.003006169 |
| MARS | 0.5913011 | 0.003090662 |
| CREBZF | 0.41304496 | 0.003133359 |
| R3HCC1 | 0.46226665 | 0.003176317 |
| RP11-108M9.4 | −0.1341357 | 0.003218726 |
| CEBPZOS | 0.040552 | 0.003304363 |
| RPRD1B | 0.6943582 | 0.003459569 |
| ARL16 | 0.2653141 | 0.003487473 |
| SMPD1 | 0.75771 | 0.003488205 |
| GLS | 0.23158617 | 0.003570829 |
| ZBTB18 | 0.44361597 | 0.003811838 |
| DHRSX | 0.41621807 | 0.00402441 |
| MVD | 0.4788746 | 0.004534107 |
| ARRDC2 | 0.28106672 | 0.004698064 |
| KANSL1-AS1 | 0.31124815 | 0.00492885 |
| CPNE8 | 0.36672577 | 0.005019354 |
| CTD-3184A7.4 | 0.16273294 | 0.00535751 |
| ADCK4 | 1.0378616 | 0.005367539 |
| MMP25 | 1.2973027 | 0.005406462 |
| TMED4 | 0.07271768 | 0.00541181 |
| RP11-83A24.2 | 0.11481906 | 0.005424892 |
| SPON2 | 0.44224834 | 0.005658111 |
| FAM199X | 0.68025035 | 0.005684569 |
| DENND1C | 0.41338673 | 0.005690967 |
| FCF1 | 0.60417473 | 0.005806388 |
| FINC00937 | 0.20855135 | 0.005830548 |
| UPF1 | 0.7624002 | 0.005830548 |
| FAM184B | 0.28910863 | 0.005855788 |
| TRPM2 | 0.6475276 | 0.005866325 |
| AC245100.1 | 0.43681246 | 0.005916072 |
| CASP2 | 0.19648436 | 0.006043118 |
| EIF2S3 | −0.104916826 | 0.006048061 |
| SGSM3 | 0.4147272 | 0.006104934 |
| CDKN1C | 0.24942796 | 0.006153362 |
| RP11-841020.2 | 0.31913427 | 0.006316818 |
| ENG | 0.5683443 | 0.006352306 |
| TP53I11 | 1.0254523 | 0.00637664 |
| ELMSAN1 | 0.18789494 | 0.006472349 |
| MOGS | 0.4225218 | 0.006590462 |
| FAM98C | 0.32311133 | 0.006774202 |
| TXLNA | 0.5911916 | 0.006819017 |
| TCF4 | 0.50261575 | 0.007280694 |
| FUT7 | 0.95993096 | 0.007438439 |
| RGL2 | 0.23456806 | 0.008110767 |
| FLAD1 | 0.35986292 | 0.008135388 |
| MROH1 | 0.5935402 | 0.008297022 |
| C5orf56 | 0.08737781 | 0.008630567 |
| THAP7 | 0.3141596 | 0.008680306 |
| C12orf75 | 0.25778544 | 0.008680306 |
| GZMM | 0.12528384 | 0.00876589 |
| RFFL | 0.45862398 | 0.008912474 |
| PSIP1 | 0.20980138 | 0.008944487 |
| PRKD2 | 0.7431864 | 0.009015754 |
| WDR6 | 0.41467637 | 0.009031811 |
| PI4KA | 0.23824759 | 0.009379663 |
| LINC01410 | 0.30084297 | 0.009422757 |
| WTAP | −0.14992253 | 0.009480682 |
| MAFK | 0.74858445 | 0.009639682 |
| C16orf54 | −0.078989804 | 0.010374847 |
| SELO | 0.38543004 | 0.010489496 |
| SH3GL1 | 0.58258075 | 0.010881156 |
| RSRC1 | 0.11351054 | 0.010992583 |
| TMEM63A | 0.90585786 | 0.011127617 |
| LCK | 0.25730258 | 0.011859401 |
| C16orf70 | 0.72893107 | 0.012147367 |
| MXD3 | 0.1662068 | 0.013067787 |
| RP3-477O4.14 | 0.5024213 | 0.013141718 |
| RING1 | 0.30966774 | 0.013349354 |
| ZMIZ2 | 0.4110426 | 0.014289953 |
| BOLA2B | 0.017815607 | 0.014694429 |
| DGAT1 | 0.22785929 | 0.01500983 |
| FBXO18 | 0.48248273 | 0.015063613 |
| ZFX | 0.12325215 | 0.015657906 |
| PHKA2 | 0.64061785 | 0.015873583 |
| ATG9A | 0.572491 | 0.015873583 |
| IRF2BPL | −0.10120596 | 0.015875152 |
| PLCL2 | 0.37231326 | 0.015946915 |
| TPCN2 | 0.7053937 | 0.0162729 |
| TMEM55B | 0.36833435 | 0.016337855 |
| TCEAL4 | 0.33991665 | 0.018475807 |
| CPSF1 | 0.6040196 | 0.019478916 |
| ZNF335 | 0.61582196 | 0.019695754 |
| DHRS4 | 0.1717924 | 0.019784151 |
| EXOSC6 | 0.107335344 | 0.020178846 |
| PTGDS | 0.76167935 | 0.020645823 |
| LINC01001 | 0.3257109 | 0.021832168 |
| PGS1 | 0.42874464 | 0.023057214 |
| TMEM192 | 0.22923602 | 0.023368905 |
| LIPE-AS1 | −0.018458713 | 0.024283753 |
| ZNF276 | 0.29647613 | 0.025413806 |
| MIER2 | 0.79632545 | 0.025756717 |
| SEC22B | −0.16659798 | 0.026620585 |
| DDIT3 | 0.4095274 | 0.026974976 |
| CLEC2D | 0.16468048 | 0.027126574 |
| PDPR | 0.49912882 | 0.02796406 |
| RP11-443B7.1 | 0.35282224 | 0.028212229 |
| CC2D1A | 0.6483858 | 0.028711863 |
| CLK2 | 0.590695 | 0.02945504 |
| GPS2 | 0.120389976 | 0.029716424 |
| ZC3H4 | −0.008363298 | 0.030503053 |
| IL1B | 0.8685812 | 0.032146482 |
| WDR74 | 0.07998445 | 0.032845134 |
| P2RY8 | 0.3444206 | 0.032934183 |
| PPP1R3B | 0.7779614 | 0.033100016 |
| METTL17 | 0.5690176 | 0.033586027 |
| LETM1 | 0.14687458 | 0.033763208 |
| TSC2 | 0.40044802 | 0.033888253 |
| FAM118A | 0.32594064 | 0.03444318 |
| TAOK2 | 0.63447666 | 0.03491745 |
| ARID1B | 0.16305847 | 0.035244774 |
| FSTL3 | 0.7042341 | 0.035565256 |
| SHROOM1 | 0.4680259 | 0.036160169 |
| NFKB2 | 0.29089114 | 0.036411936 |
| KMT5A | 0.40287706 | 0.037537004 |
| TSPAN32 | −6.67E−05 | 0.037541023 |
| GIT1 | 0.54060024 | 0.038965378 |
| AP5Z1 | 0.4205903 | 0.039265916 |
| CD8B | 0.23486653 | 0.04096929 |
| TPCN1 | 0.3642896 | 0.042780013 |
| TLCD2 | 0.6894843 | 0.043135357 |
| EZH1 | 0.3072416 | 0.044053852 |
| ZNF384 | 0.6672497 | 0.044523986 |
| CTD-3252C9.4 | 0.12243309 | 0.045120169 |
| SEMA4B | 0.67897993 | 0.045468325 |
| BRD8 | 0.3735951 | 0.04613623 |
| COG4 | 0.42823148 | 0.046874408 |
| ACP5 | 0.100093134 | 0.046949303 |
| MICA | 0.5462366 | 0.047166533 |
| CELA1 | 0.83215505 | 0.048019758 |
| TRMT2A | 0.32218897 | 0.048605988 |
| C9orf139 | 0.24184373 | 0.048640082 |
| USP19 | 0.8140703 | 0.049380431 |
| CLASRP | 0.15063801 | 0.049380431 |
| PACS1 | 0.3236276 | 0.050484947 |
| IGLC3 | 0.5456555 | 0.050484947 |
| RUSC1 | 0.5074146 | 0.053515003 |
| KMT2B | 0.58046925 | 0.054094371 |
| HAL | 0.5761656 | 0.054428359 |
| CCL3 | 0.30250442 | 0.05460007 |
| PTPN7 | 0.6627701 | 0.055882213 |
| JUN | 0.57463634 | 0.055930576 |
| RPS6KA5 | 0.14065544 | 0.056810387 |
| RASSF7 | 0.15741393 | 0.057160127 |
| GPR160 | 0.776766 | 0.057524657 |
| TIPIN | 0.060413145 | 0.057635503 |
| KCNJ2 | 0.044291418 | 0.057913418 |
| CDC25B | 0.43588713 | 0.057942074 |
| C1QC | 1.1810297 | 0.059826999 |
| SGSH | 0.66092485 | 0.060301644 |
| CD247 | 0.24086288 | 0.062005277 |
| RP11-67L2.2 | 0.6772472 | 0.062424146 |
| PCNXL3 | 0.59625834 | 0.062457775 |
| PLA2G15 | 0.62968165 | 0.062912239 |
| IFI27 | 2.4521592 | 0.063525546 |
| CAPN10 | 0.6063863 | 0.06519955 |
| ZC3H7B | 0.7752986 | 0.068291537 |
| SMURF2 | 0.4942112 | 0.071617049 |
| DGKA | 0.42396876 | 0.072213425 |
| UQCRHL | −0.112802446 | 0.072351139 |
| WDR83 | 0.43120322 | 0.074120085 |
| BCR | 0.7780288 | 0.074169921 |
| POMC | 0.16960849 | 0.074944914 |
| ATP5L2 | −0.11425391 | 0.075199569 |
| RP11-294J22.6 | 0.11993532 | 0.076033962 |
| ACTR1B | 0.27123865 | 0.076335191 |
| SLC16A5 | 0.5762419 | 0.077049986 |
| PICK1 | 1.0061951 | 0.078574813 |
| MLLT6 | 0.3097411 | 0.0794262 |
| PACS2 | 0.4270651 | 0.079958246 |
| SNHG9 | −0.21151823 | 0.081772294 |
| TPT1-AS1 | 0.47170144 | 0.084967119 |
| STRN3 | −0.004971773 | 0.0870804 |
| RNF2 | 0.001323912 | 0.087738074 |
| METTL12 | 0.03283119 | 0.08830219 |
| MAPK8IP3 | 0.35840976 | 0.088789508 |
| MADD | 0.3468371 | 0.091194315 |
| CTC-246B18.10 | 0.14886552 | 0.094390407 |
| LRRC75A | 0.10333958 | 0.095691618 |
| RP5-1171I10.5 | −0.1719408 | 0.096956243 |
| MEPCE | 0.2890762 | 0.09826087 |
| CTD-2017F17.2 | 0.3365356 | 0.099077553 |

n = 15,021, 11,439, 21,386, and 43,536 cells, for MS1, MS2, MS4, and all other monocytes, respectively
FDR values are computed with a two-sided Wilcoxon rank-sum test with Benjami-Hochberg correction

TABLE 3
Deconvolution Meta-analysis Results

| | | | | | | |
|---|---|---|---|---|---|---|
| | | Effect Size | Effect Size | Tau | | Heterogeneity |
| State | Effect Size | Standard Error | FDR | Squared | Cochrane's Q | P-value |

Sepsis vs. Healthy Controls
n = 775 total patients from 11 cohorts; Meta-analysis
results are the outputs from the R package, MetaIntegrator

| | | | | | | |
|---|---|---|---|---|---|---|
| MS1 | 1.90E+00 | 1.64E−01 | 1.75E−30 | 1.61E−01 | 2.66E+01 | 3.03E−03 |
| BS1 | 1.45E+00 | 1.24E−01 | 1.75E−30 | 6.72E−02 | 1.78E+01 | 5.76E−02 |
| MS3 | 4.60E−01 | 1.54E−01 | 2.99E−03 | 1.59E−01 | 3.23E+01 | 3.60E−04 |
| MS4 | 2.65E−02 | 1.35E−01 | 8.44E−01 | 1.07E−01 | 2.52E+01 | 4.93E−03 |
| BS2 | −1.14E+00 | 2.60E−01 | 1.38E−05 | 5.98E−01 | 8.31E+01 | 1.23E−13 |
| MK | −1.30E+00 | 1.65E−01 | 5.62E−15 | 1.83E−01 | 3.18E+01 | 4.34E−04 |
| DS1 | −1.35E+00 | 1.62E−01 | 1.80E−16 | 1.72E−01 | 3.03E+01 | 7.75E−04 |
| BS3 | −1.48E+00 | 1.64E−01 | 4.90E−19 | 1.75E−01 | 2.97E+01 | 9.47E−04 |
| TS2 | −1.55E+00 | 1.51E−01 | 4.74E−24 | 1.35E−01 | 2.54E+01 | 4.70E−03 |
| DS2 | −1.66E+00 | 2.28E−01 | 4.05E−13 | 4.25E−01 | 5.56E+01 | 2.39E−08 |
| TS3 | −1.75E+00 | 1.87E−01 | 1.97E−20 | 2.48E−01 | 3.60E+01 | 8.34E−05 |
| NS1 | −1.78E+00 | 2.38E−01 | 8.59E−14 | 4.61E−01 | 5.77E+01 | 1.00E−08 |
| NS2 | −1.82E+00 | 2.41E−01 | 6.40E−14 | 4.77E−01 | 5.88E+01 | 6.10E−09 |
| MS2 | −1.83E+00 | 2.20E−01 | 2.29E−16 | 3.86E−01 | 5.04E+01 | 2.26E−07 |
| DS3 | −1.99E+00 | 2.43E−01 | 4.39E−16 | 4.84E−01 | 5.66E+01 | 1.56E−08 |
| TS1 | −2.18E+00 | 1.87E−01 | 1.75E−30 | 2.29E−01 | 3.15E+01 | 4.92E−04 |

Sepsis vs. Sterile Inflammation
n = 696 total patients from 7 cohorts; Meta-analysis
results are the outputs from the R package, MetaIntegrator

| | | | | | | |
|---|---|---|---|---|---|---|
| MS3 | 3.47E−01 | 1.68E−01 | 1.25E−01 | 1.28E−01 | 1.91E+01 | 3.92E−03 |
| MS1 | 3.23E−01 | 1.29E−01 | 8.64E−02 | 5.28E−02 | 1.15E+01 | 7.54E−02 |
| TS2 | 1.56E−02 | 8.89E−02 | 9.70E−01 | 0.00E+00 | 4.57E+00 | 6.00E−01 |
| MS4 | 3.15E−04 | 1.77E−01 | 9.99E−01 | 1.49E−01 | 2.14E+01 | 1.56E−03 |
| MK | −1.77E−02 | 1.56E−01 | 9.70E−01 | 1.02E−01 | 1.66E+01 | 1.08E−02 |
| TS1 | −3.85E−02 | 1.12E−01 | 9.36E−01 | 2.77E−02 | 8.89E+00 | 1.80E−01 |
| BS1 | −4.76E−02 | 1.56E−01 | 9.36E−01 | 1.05E−01 | 1.69E+01 | 9.74E−03 |
| NS2 | −5.06E−02 | 1.23E−01 | 9.36E−01 | 4.37E−02 | 1.06E+01 | 1.03E−01 |
| BS2 | −7.72E−02 | 1.50E−01 | 9.36E−01 | 9.07E−02 | 1.54E+01 | 1.72E−02 |
| BS3 | −9.83E−02 | 1.52E−01 | 9.23E−01 | 9.59E−02 | 1.59E+01 | 1.42E−02 |
| DS3 | −1.78E−01 | 1.58E−01 | 5.23E−01 | 1.08E−01 | 1.71E+01 | 8.81E−03 |
| DS2 | −2.44E−01 | 1.44E−01 | 2.07E−01 | 7.95E−02 | 1.42E+01 | 2.74E−02 |
| TS3 | −2.47E−01 | 1.35E−01 | 1.80E−01 | 6.29E−02 | 1.25E+01 | 5.19E−02 |
| DS1 | −2.99E−01 | 1.30E−01 | 8.64E−02 | 5.41E−02 | 1.16E+01 | 7.19E−02 |
| NS1 | −3.34E−01 | 1.45E−01 | 8.64E−02 | 8.13E−02 | 1.44E+01 | 2.57E−02 |
| MS2 | −3.92E−01 | 8.95E−02 | 1.94E−04 | 0.00E+00 | 3.31E+00 | 7.69E−01 |

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba in the present disclosure. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated in the present disclosure by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly in the present disclosure. Any particular embodiment of the invention can be excluded from any claim, for any reason, whether or not related to the existence of prior art.

Those skilled in the art will recognize or be able to ascertain using no more than routine experimentation many equivalents to the specific embodiments described in the present disclosure. The scope of the present embodiments described in the present disclosure is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method for treating a subject for sepsis, comprising:
administering an antibiotic to a subject who has been identified as having elevated levels of CD45+ monocytes that are IL1R2^{hi}, HLA-DR^{lo}, and CD14+ relative to a control.

2. A method for treating a subject for sepsis, comprising:
identifying a subject as having elevated levels of CD45+ monocytes that are IL1R2^{hi}, HLA-DR^{lo}, and CD14+ relative to a control; and
administering an antibiotic to the subject.

3. A method comprising:
measuring the fraction of CD45+ monocytes that are IL1R2^{hi}, HLA-DR^{lo}, and CD14+ in a blood sample from a subject; and
comparing the fraction of CD45+ monocytes that are IL1R2^{hi}, HLA-DR^{lo}, and CD14+ in the blood sample from the subject to a control.

4. A method for determining whether a subject has bacterial sepsis, comprising
measuring the fraction of CD45+ monocytes that are IL1R2^{hi}, HLA-DR^{lo}, and CD14+ in a blood sample from the subject;
comparing the fraction of CD45+ monocytes that are IL1R2^{hi}, HLA-DR^{lo}, and CD14+ in the blood sample from the subject to a control; and
determining that the subject has bacterial sepsis if the fraction of CD45+ monocytes that are IL1R2^{hi}, HLA-DR^{lo}, and CD14+ in the blood sample from the subject is elevated compared to the control.

5. The method of claim 3, further comprising determining that the subject has bacterial sepsis if the fraction of CD45+ monocytes that are IL1R2^{hi}, HLA-DR^{lo}, and CD14+ in the blood sample from the subject is elevated compared to a control.

6. The method of any one of claims 1-5, wherein the control is a blood sample from a healthy subject.

7. The method of any one of claims 1-5, wherein the control is a predetermined value.

8. The method of any one of claims 3-7, further comprising administering an antibiotic to the subject.

9. The method of claim 1 or claim 2, wherein identifying a subject as having elevated levels of CD45+ monocytes that are IL1R2^{hi}, HLA-DR^{lo}, and CD14+ relative to a control comprises conducting an RNA-sequencing assay.

10. The method of any one of claims 3 to 8, wherein measuring the fraction of CD45+ monocytes that are IL1R2^{hi}, HLA-DR^{lo}, and CD14+ comprises conducting an RNA-sequencing assay.

11. The method of claim 9 or claim 10, wherein the RNA-sequencing assay comprises a single cell RNA-sequencing (scRNA-seq) assay.

12. The method of claim 1 or claim 2, wherein identifying a subject as having elevated levels of CD45+ monocytes that are IL1R2^{hi}, HLA-DR^{lo}, and CD14+ relative to a control comprises conducting a flow cytometry assay.

13. The method of any one of claims 3 to 8, wherein measuring the fraction of CD45+ monocytes that are IL1R2^{hi}, HLA-DR^{lo}, and CD14+ comprises conducting a flow cytometry assay.

14. The method of claim 12 or claim 13, wherein the flow cytometry assay comprises a fluorescence activated cell sorting (FACS) assay.

15. The method of any one of claims 3 to 14, wherein the blood sample comprises total CD45+ monocytes and enriched dendritic cells.

16. The method of any one of claims 3 to 15, wherein the blood sample is obtained from a human.

17. The method of any one of claims 1 to 16, wherein the subject is a human patient having, suspected of having, or at risk for a bacterial infection.

18. The method of any one of claims 1 to 16, wherein the subject is a human patient having, suspected of having, or at risk for bacterial sepsis.

19. The method of claim 17, wherein the bacterial infection is associated with a bacteria selected from the group consisting of Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria; Staphylococcus; Streptococcus; and Salmonella.

20. The method of claim 18, wherein the bacterial sepsis is associated with a bacteria selected from the group consisting of Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria; Staphylococcus; Streptococcus; and Salmonella.

21. The method of any one of claims 1 to 16, wherein the subject is a human patient having, suspected of having, or at risk for a urinary tract infection (UTI).

22. A method for determining whether a subject has bacterial sepsis, comprising
measuring the level of RETN, IL1R2, and/or CLU in CD14+ monocytes in a blood sample from the subject;
comparing the level of RETN, IL1R2, and/or CLU in CD14+ monocytes in the blood sample from the subject to a control; and
determining that the subject has bacterial sepsis if the level of RETN, IL1R2, and/or CLU in CD14+ monocytes in the blood sample from the subject is elevated relative to a control.

23. A method of identifying a sepsis condition in a subject comprising identifying an elevated fraction of MS1 type monocytes in the subject.

24. A method of identifying and treating a sepsis condition in a subject comprising identifying an elevated fraction of MS1 type monocytes in the subject, and treating the subject having elevated MS1 type monocytes by administering one or more antibiotic agents to the subject.

25. The method of claim 23 or 24, wherein the MS1 type monocytes are CD14+ monocytes characterized by high expression of RETN, IL1R2, and CLU.

26. A method for generating MS1 type monocytes, comprising: incubating CD34+ bone marrow mononuclear cells (BMMCs) in the presence of IL6 and/or IL10.

27. The method of claim 26, wherein the CD34+ BMCs are incubated in the presence of plasma from sepsis patients.

28. The method of claim 27, wherein the CD34+ BMMCs are incubated in culture media that comprises approximately 20% plasma from sepsis patients.

29. The method of any one of claims 26-28, wherein the CD34+ BMMCs are incubated for at least 1, 2, 3, 4, 5, 6, 7, 8, 9, or 10 days.

30. The method of any one of claims 26-29, wherein incubation of the CD34+ BMMCs results in STAT3-Y705 phosphorylation.

31. The method of any one of claims 26-30, wherein the CD34+ BMMCs are incubated in the presence of GM-CSF and/or and M-CSF.

32. The method of any one of claims 26-31, wherein incubation of the CD34+ BMMCs results in upregulation of expression of one or more of: S100A8, S100A12, VCAN, RETN, LYZ, MNDA, CTSD, SELL, CYP1B1, CLU, NKG7, MCEMP1, TIMP1, SOD2, CD163, NAMPT, ACSL1, VAMP5, LILRA5, VNN2, ANXA6, CALR, and CTSA compared with CD34+ HSPCs incubated in the presence of plasma from heathy subjects.

33. The method of claim 32, wherein incubation of the CD34+ BMMCs results in upregulation of expression of S100A8, MNDA, and VCAN compared with CD34+ HSPCs incubated in the presence of plasma from heathy subjects.

34. The method of any one of claims 26-33, wherein the BMMCs are hematopoietic stem and progenitor cells (HSPCs).

35. The method of any one of claims 26-34, wherein the CD34+ BMMCs are derived from bone marrow.

36. The method of claim 34, wherein the HSPCs are derived from cord blood.

37. The method of claim 34, wherein the HSPCs are derived from peripheral blood.

38. The method of any one of claims 26-37, wherein the CD34+ BMMCs are incubated ex vivo.

39. The method of claim 38, wherein the CD34+ BMMCs are administered to a subject following incubation.

40. The method of claim 39, wherein the subject has autoimmunity, infectious immunity with a cytokine storm, transplant rejection, and/or sepsis.

41. The method of claim 40, wherein the CD34+ BMMCs are administered to the same subject from whose bone marrow the CD34+ HSPCs were derived.

42. The method of any one of claims 26-38, wherein the MS1 type monocytes are used for screening for therapeutics.

43. The method of claim 42, wherein the therapeutic is an inducer of MS1 type monocytes.

44. The method of claim 42, wherein the therapeutic is an inhibitor of MS1 type monocytes.

45. The method of any one of claims 26-44, wherein the incubation of the MS1 type monocytes delays and/or suppresses the proliferation of CD4 T cells.

46. The method of any one of claims 26-44, wherein the incubation of the MS1 type monocytes delays and/or suppresses the proliferation of CD8 T cells.

47. The method of claim 44 or claim 46 further comprising CD3 and CD28.

48. The method of any one of claims 26-44, wherein the incubation of the MS1 type monocytes results in upregulation of expression of MMP1, PROS1, VCAM1, SST, and FN1.

49. The method of any one of claims 26-44, wherein the incubation of the MS1 type monocytes results in suppression of inflammatory cytokine gene expression.

50. The method of claim 49, wherein the incubation of the MS1 type monocytes results in suppression of one or more of: BIRC3, CXCL8, CSF2, CXCL1, ID3, CCL2, and NFKBIA compared with MS1 type monocytes incubated in the presence of sepsis serum.

51. The method of claim 49 or claim 50 further comprising sepsis serum.

52. The method of any one of claims 26-51, wherein the culture media of MS1 type monocytes results in the suppression of the upregulation of chemokine genes.

53. The method of claim 52, wherein the chemokine genes are associated with the cytokine-cytokine receptor interaction, NOD-like receptor signaling pathway, and/or pathways in cancer.

54. The method of any one of claims 26-53, wherein the MS1 type monocytes comprise elevated levels of ARG1, iNOS, and/or ROS.


SEQUENCE LISTING (6 sequences)

SEQ ID NO: 1 - DNA, 516 bp, Homo sapiens
aagaggcctcaaagaaagagctgcggtgcaggaattcgtgtgccggatttggttagctgagcccaccgagaggcgcctgcaggatgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagcgcgtgcacagcgcgggcggaggcggctccaggtccggaggggttgcgggggagctggaaataaacctggagatgatgatgatgatgatgatga

SEQ ID NO: 2 - DNA, 507 bp, Homo sapiens
aagaggcctcaaagaaagagctgcggtgcaggaattcgtgtgccggatttggttagctgagcccaccgagagggatgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagcgcgtgcacagcgcgggcggaggcggctccaggtccggaggggttgcgggggagctggaaataaacctggagatgatgatgatgatgatgatga

SEQ ID NO: 3 - DNA, 1462 bp, Homo sapiens
gctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaactccgtacgtcttctcttatggaagtggctgtgtctttttgagggactctgttctttgcctcagttgtctaccaaaggtgccacatttatagtggctttgtagtaaaggactaaagtctta

SEQ ID NO: 4 - DNA, 1463 bp, Homo sapiens
gcagagtggcacagtcacattctagaagaccatgtgggatgggagatactgttgtggtcacctctggaaaatacattctgctactcttaaaaactagtgacgctcatacaaatcaacagaaagagcttctgaaggaagactttaaagctgcttctgccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaa

SEQ ID NO: 5 - DNA, 1158 bp, Homo sapiens
gctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccagtaagtgggccagggtcttctgttgagaactctgtgggtttcgctcttccttttggagacagttatcactatgacccacataccacattaaaagttactttttttgattccaaactgttggatgtttagaatttaaaaaattgtattttgctaaaaat

SEQ ID NO: 6 - DNA, 2749 bp, Homo sapiens
gcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcacactgtttcaaaagtta